(19) United States
(12) Patent Application Publication
(10) Pub. No.: US 2023/0331654 A1
(43) Pub. Date: Oct. 19, 2023

(54) COMPOUND FOR ORGANIC ELECTRIC DEVICE, ORGANIC ELECTRIC DEVICE USING SAME, AND ELECTRONIC DEVICE THEREOF

(71) Applicant: DUK SAN NEOLUX CO., LTD., Cheonan-si, Chungcheongnam-do (KR)

(72) Inventors: Jin Ho YUN, Cheonan-si, Chungcheongnam-do (KR); Yun Jong KO, Cheonan-si, Chungcheongnam-do (KR); Yong Wook PARK, Cheonan-si, Chungcheongnam-do (KR); Sun Hee LEE, Cheonan-si, Chungcheongnam-do (KR); Soung Yun MUN, Cheonan-si, Chungcheongnam-do (KR); Byoung Yeop KANG, Cheonan-si, Chungcheongnam-do (KR)

(73) Assignee: DUK SAN NEOLUX CO., LTD., Cheonan-si, Chungcheongnam-do (KR)

(21) Appl. No.: 17/760,042

(22) PCT Filed: Feb. 25, 2021
(86) PCT No.: PCT/KR2021/001655

§ 371 Date: Aug. 3, 2022

(30) Foreign Application Priority Data
Feb. 10, 2020 (KR) 10-2020-0015656

(51) Int. Cl. C07C 211/61; C07D 209/88; C07D 333/76; C07D 307/91; H10K 50/11; H10K 50/81; H10K 50/82; H10K 50/17; H10K 50/16; H10K 50/15; H10K 85/60

(52) U.S. Cl. CPC C07C 211/61; C07D 209/88; C07D 333/76; C07D 307/91; H10K 50/11; H10K 50/81; H10K 50/82; H10K 50/171; H10K 50/16; H10K 50/15; H10K 85/633

(57) ABSTRACT

Provided are an OLED compound, an organic electronic element employing the compound, and an electronic device comprising the element, where the compound improves the luminous efficiency, stability and lifetime of the organic electronic element.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer. This is because, when the energy level and T1 value between each organic material layer, and the intrinsic properties of the material (mobility, interfacial properties, etc.) are optimally combined, a long lifespan and high efficiency can be achieved at the same time.

Also, in order to solve the problem of light emission in the hole transport layer in recent organic electroluminescent devices, an emitting-auxiliary layer must exist between the hole transport layer and the emitting layer, and it is time to develop different emitting-auxiliary layers according to each emitting layer (R, G, B).

In general, electrons are transferred from the electron transport layer to the emitting layer, and holes are transferred from the hole transport layer to the emitting layer, and excitons are generated by recombination.

However, since the material used for the hole transport layer should have a low HOMO value, most have a low T1 value. As a result, excitons generated in the emitting layer are transferred to the hole transport layer, resulting in charge unbalance in the emitting layer to emit light at the hole transport layer interface.

When light is emitted at the hole transport layer interface, the color purity and efficiency of the organic electronic element are lowered, and the lifespan is shortened. Therefore, it is urgently required to develop an emitting-auxiliary layer having a high T1 value and having a HOMO level between the HOMO energy level of the hole transport layer and the HOMO energy level of the emitting layer.

Furthermore, it is necessary to develop a hole injection layer material that delays the penetration and diffusion of metal oxides from the anode electrode (ITO) into the organic layer, which is one of the causes of shortening the lifespan of organic electronic element, and that has stable characteristics, that is, a high glass transition temperature, even against Joule heating generated during device driving. The low glass transition temperature of the hole transport layer material has a characteristic of lowering the uniformity of the thin film surface during device driving, which is reported to have a significant effect on device lifespan. Moreover, OLED devices are mainly formed by a deposition method, and it is necessary to develop a material that can withstand a long time during deposition, that is, a material with strong heat resistance.

In other words, in order to fully exhibit the excellent characteristics of an organic electronic element, it should be preceded that the material constituting the organic material layer in the device, such as a hole injection material, a hole transport material, a light emitting material, an electron transport material, an electron injection material, emitting auxiliary layer material, etc., is supported by a stable and efficient material, but the development of a stable and efficient organic material layer material for an organic electronic device has not yet been sufficiently made. Therefore, the development of new materials is continuously required.

As a reference prior art document, KR1020130076842 A was used.

### DETAILED DESCRIPTION OF THE INVENTION

### Summary

In order to solve the problems of the above-mentioned background art, the present invention has revealed a compound having a novel structure, and when this compound is applied to an organic electronic element, it has been found that the luminous efficiency, stability and lifespan of the device can be significantly improved.

Accordingly, an object of the present invention is to provide a novel compound, an organic electronic element using the same, and an electronic device thereof.

### Technical Solution

The present invention provides a compound represented by Formula (1).

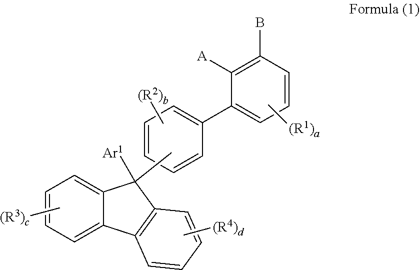

In another aspect, the present invention provides an organic electronic element comprising the compound represented by Formula (1) and an electronic device thereof.

### Effects of the Invention

By using the compound according to the present invention, high luminous efficiency, low driving voltage and high heat resistance of the device can be achieved, and color purity and lifespan of the device can be greatly improved.

### BRIEF DESCRIPTION OF THE DRAWINGS

FIG. 1 to FIG. 3 are exemplary views of an organic electroluminescent device according to the present invention.

FIG. 4 shows a Formula according to an aspect of the present invention.

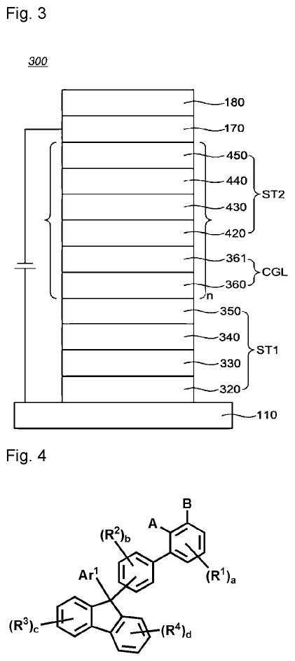

| | |
|---|---|
| 100, 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

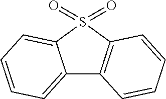 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

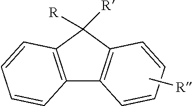

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro’ and ‘tri-spiro’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

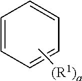

Here, when a is an integer of 0, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

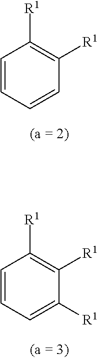

Hereinafter, a compound according to an aspect of the present invention and an organic electronic element including the same will be described.

The present invention provides a compound represented by Formula (1).

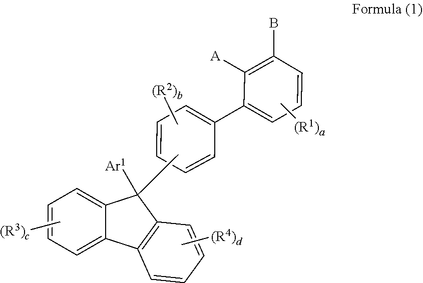

{In Formula (1), each symbol may be defined as follows.

1) R¹, R², R³, R⁴, A and B are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or in case a, b, c and d are 2 or more, R¹, R², R³ and R⁴ are each in plural being the same or different, a plurality of adjacent R¹s, or a plurality of R²s, or a plurality of R³s, or a plurality of R⁴s may be bonded to each other to form a ring.

Wherein in case R¹, R², R³, R⁴, A and B are an alkyl group, it may be preferably a C₁-C₃₀ alkyl group, and more preferably a C₁-C₂₄ alkyl group.

In case R¹, R², R³, R⁴, A and B are an alkenyl group, they may be preferably an C₂-C₃₀ alkenyl group, more preferably an C₂-C₂₄ alkenyl group.

In case R¹, R², R³, R⁴, A and B are an alkynyl group, they may be preferably an C₂-C₃₀ alkynyl group, more preferably an C₂-C₂₄ alkynyl group.

In case R¹, R², R³, R⁴, A and B are an alkoxyl group, it may be preferably a C₁-C₃₀ alkoxyl group, more preferably an C₁-C₂₄ alkoxyl group.

In case R¹, R², R³, R⁴, A and B are an aryloxy group, it may be preferably a C₆-C₃₀ aryloxy group, more preferably an C₆-C₂₄ aryloxy group.

In case R¹, R², R³, R⁴, A and B are an aryl group, it may be preferably a C₆-C₃₀ aryl group, and more preferably a C₆-C₂₅ aryl group, for example, it may be phenylene, biphenyl, naphthalene, terphenyl, etc.

In case R¹, R², R³, R⁴, A and B are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case R¹, R², R³, R⁴, A and B are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R¹, R², R³, R⁴, A and B are an arylamine group, it may be preferably a C₆-C₃₀ arylamine group, and more preferably a C₆-C₂₄ arylamine group,

2) However, at least one of A and B is a substituent represented by Formula (A),

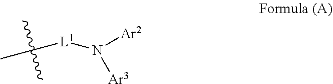

3) a is an integer from 0 to 3, b, c and d are each independently an integer from 0 to 4,

4) Ar¹ is selected from the group consisting of a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;

Wherein in case Ar¹ is an alkyl group, it may be preferably a C₁-C₃₀ alkyl group, and more preferably a C₁-C₂₄ alkyl group.

In case Ar¹ is an alkoxyl group, it may be preferably an C₁-C₂₄ alkoxyl group.

In case Ar¹ is an aryloxy group, it may be preferably a C₆-C₂₄ aryloxy group.

In case Ar¹ is an aryl group, it may be preferably a C₆-C₃₀ aryl group, and more preferably a C₆-C₂₅ aryl group, for example, it may be phenylene, biphenyl, naphthalene, terphenyl, etc.

In case Ar¹ is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case Ar¹ is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In Formula (A),

5) L¹ is a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group;

In case L¹ is an arylene group, it may be preferably a C₆-C₃₀ arylene group, more preferably a C₆-C₂₄ arylene group, for example, phenylene, biphenyl, naphthalene, terphenyl, etc.

In case L′ is fused ring groups, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case L′ is a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

6) Ar² and Ar³ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or Ar² and Ar³ may be bonded to each other to form a ring.

In case Ar² and Ar³ are an aryl group, it may be preferably a C₆-C₃₀ aryl group, and more preferably a C₆-C₂₅ aryl group, for example, it may be phenylene, biphenyl, naphthalene, terphenyl, etc.

In case Ar² and Ar³ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case Ar² and Ar³ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

7) [symbol] represents a position bonded to Formula (1),

8) wherein the aryl group, arylene group, arylamine group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and -L′-N(Rₐ)(R_{b}); (wherein L′ is selected from the group consisting of a single bond; an C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring, and Rₐ and R_{b} are each independently selected from the group consisting of a C₆-C₆₀ aryl group; a fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;) also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, Formula (1) is represented by any one of Formulas (2) to (4)

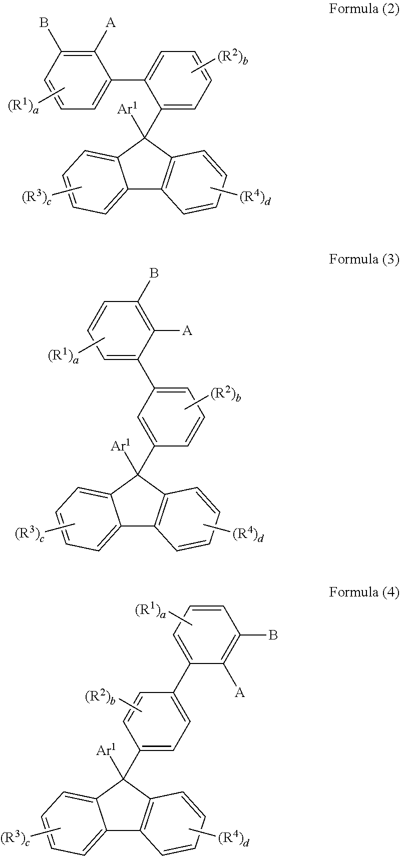

wherein R¹, R², R³, R⁴, A, B, a, b, c, d and Ar¹ are the same as defined in Formula (1) Formula (1) is represented by any one of Formulas (5) to (7)

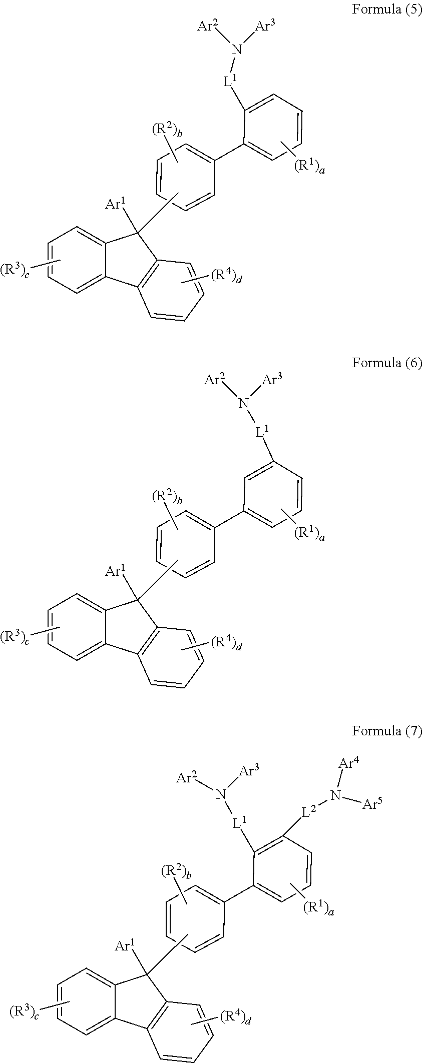

In Formulas (5) to (7), each symbol may be defined as follows.

1) R¹, R², R³, R⁴, a, b, c, d, L¹, Ar¹, Ar² and Ar³ [symbol] are the same as defined in Formula (1) and Formula (A),

2) L² is the same as the definition of L¹, provided that L¹ and L² are the same or different from each other independently,

3) Ar⁴ and Ar⁵ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or Ar⁴ and Ar⁵ may be bonded to each other to form a ring.

In case Ar⁴ and Ar⁵ are an aryl group, it may be preferably a C₆-C₃₀ aryl group, and more preferably a C₆-C₂₅ aryl group, for example, it may be phenylene, biphenyl, naphthalene, terphenyl, etc.

In case Ar⁴ and Ar⁵ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case Ar⁴ and Ar⁵ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

4) a′ is an integer of 0 to 4.

Also, Formula (1) is represented by any one of Formulas (8) to (10)

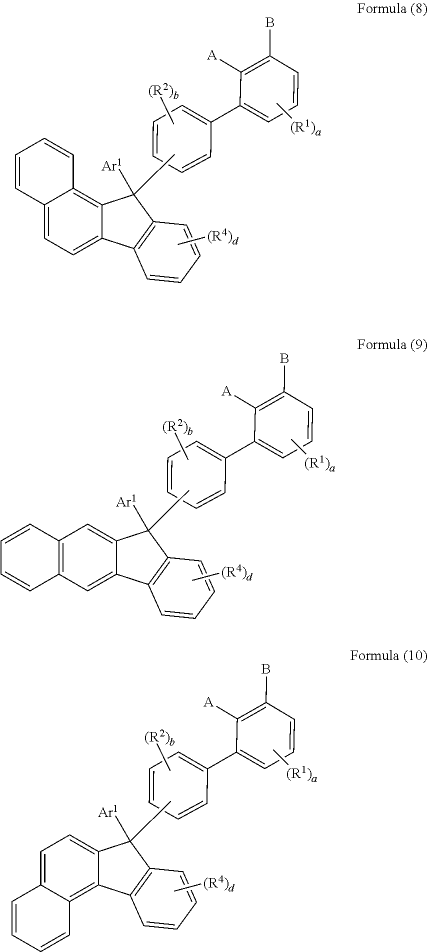

Wherein R¹, R², R⁴, A, B, a, b, d and Ar¹ are the same as defined in Formula (1).

Also, Formula (A) is represented by any one of Formulas (B) to (D)

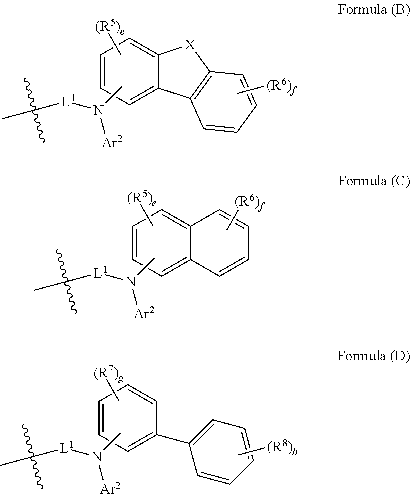

In Formulas (B) to (D), each symbol may be defined as follows.

1) L¹ and Ar² are the same as defined in Formula (1) and Formula (A).

2) X is O, S, NR′ or CR′R″,

3) R′ and R″ are each independently selected from the group consisting of hydrogen; deuterium; halogen; a C₁-C₆ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or R′ and R″ may be bonded to each other to form a ring.

Wherein in case R and R″ are an alkyl group, it may be preferably a C₁-C₃₀ alkyl group, and more preferably a C₁-C₂₄ alkyl group.

In case R′ and R″ are an alkenyl group, they may be preferably an C₂˜C₃₀ alkenyl group, more preferably an C₂˜C₂₄ alkenyl group.

In case R′ and R″ are an alkynyl group, they may be preferably an C₂-C₃₀ alkynyl group, more preferably an C₂˜C₂₄ alkynyl group.

In case R′ and R″ are an alkoxyl group, it may be preferably a C₁-C₃₀ alkoxyl group, more preferably an C₁˜C₂₄ alkoxyl group.

In case R′ and R″ are an aryloxy group, it may be preferably a C₆-C₃₀ aryloxy group, more preferably an C₆˜C₂₄ aryloxy group.

In case R′ and R″ are an aryl group, it may be preferably a C₆-C₃₀ aryl group, and more preferably a C₆-C₂₅ aryl group, for example, it may be phenylene, biphenyl, naphthalene, terphenyl, etc.

In case R′ and R″ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case R′ and R″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R′ and R″ are an arylamine group, it may be preferably a C₆-C₃₀ arylamine group, and more preferably a C₆-C₂₄ arylamine group,

4) R⁵, R⁶, R⁷ and R⁸ are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or wherein in case e, f, g and h are 2 or more, R⁵, R⁶, R⁷ and R⁸ are each in plural being the same or different, or a plurality of R⁵ or a plurality of R⁶ or a plurality of R⁷ or a plurality of R⁸ may be bonded to each other to form a ring.

Wherein in case R⁵, R⁶, R⁷ and R⁸ are an alkyl group, it may be preferably a C₁-C₃₀ alkyl group, and more preferably a C₁-C₂₄ alkyl group.

In case R⁵, R⁶, R⁷ and R⁸ are an alkenyl group, they may be preferably an C₂˜C₃₀ alkenyl group, more preferably an C₂˜C₂₄ alkenyl group.

In case R⁵, R⁶, R⁷ and R⁸ are an alkynyl group, they may be preferably an C₂˜C₃₀ alkynyl group, more preferably an C₂˜C₂₄ alkynyl group.

In case R⁵, R⁶, R⁷ and R⁸ are an alkoxyl group, it may be preferably a C₁-C₃₀ alkoxyl group, more preferably an C₁˜C₂₄ alkoxyl group.

In R⁵, R⁶, R⁷ and R⁸ are an aryloxy group, it may be preferably a C₆-C₃₀ aryloxy group, more preferably an C₆˜C₂₄ aryloxy group.

In case R⁵, R⁶, R⁷ and R⁸ are an aryl group, it may be preferably a C₆-C₃₀ aryl group, and more preferably a C₆-C₂₅ aryl group, for example, it may be phenylene, biphenyl, naphthalene, terphenyl, etc.

In case R⁵, R⁶, R⁷ and R⁸ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case R⁵, R⁶, R⁷ and R⁸ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R⁵, R⁶, R⁷ and R⁸ are an arylamine group, it may be preferably a C₆-C₃₀ arylamine group, and more preferably a C₆-C₂₄ arylamine group,

5) e is an integer from 0 to 3, f and g are each independently an integer from 0 to 4, and h is an integer from 0 to 5.

Specifically, the compound represented by Formula (1) may be any one of the following compounds 1-1 to 1-132, but is not limited thereto.

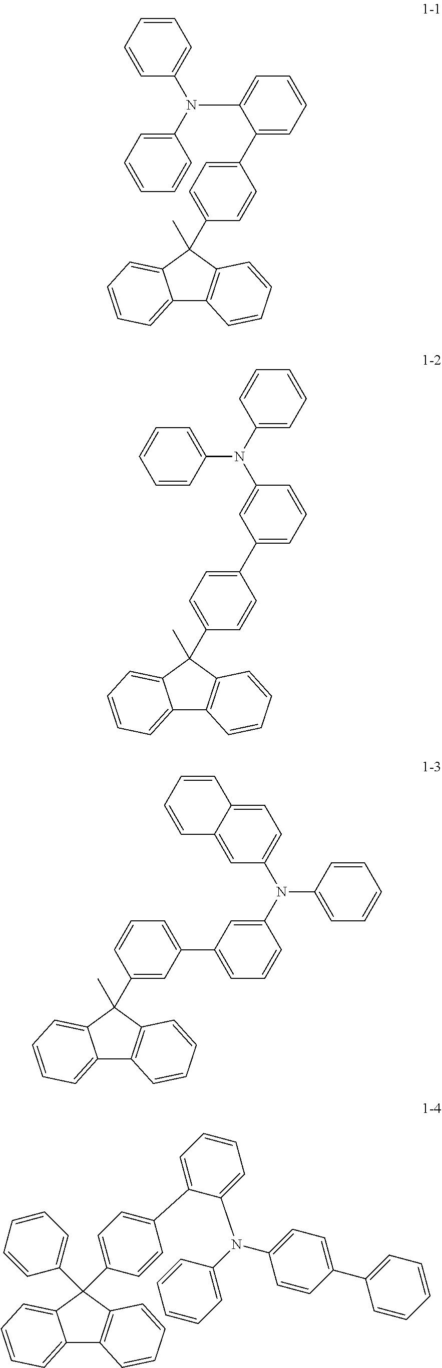

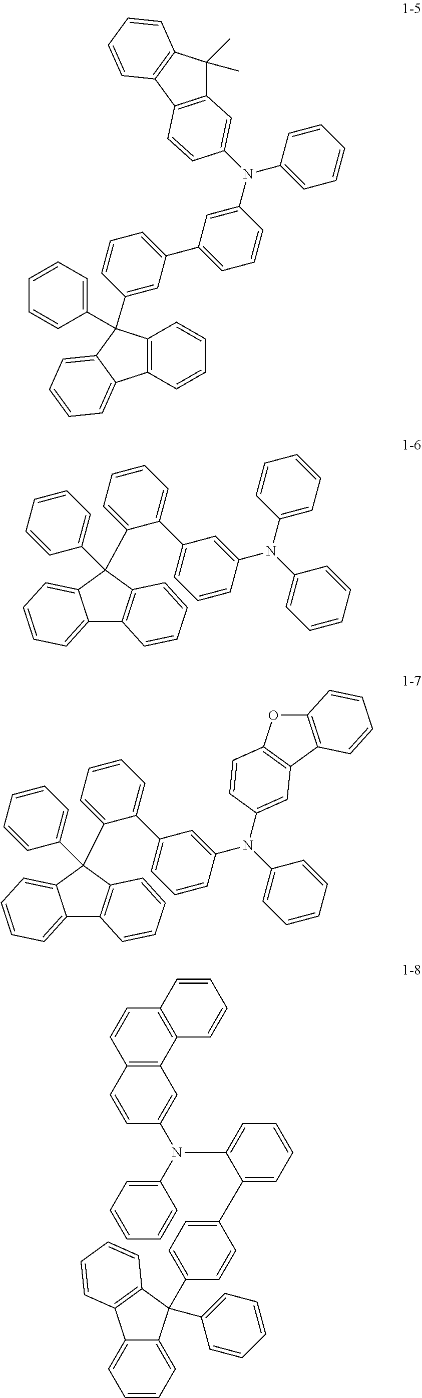 [SMILES: CC1(C)c2ccccc2-c2ccc(N(c3ccccc3)c3cccc(-c4cccc(C5(c6ccccc6)c6ccccc6-c6ccccc65)c4)c3)cc21 ; c1ccc(N(c2ccccc2)c2cccc(-c3ccccc3C3(c4ccccc4)c4ccccc4-c4ccccc43)c2)cc1 ; c1ccc(N(c2cccc(-c3ccccc3C3(c4ccccc4)c4ccccc4-c4ccccc43)c2)c2ccc3oc4ccccc4c3c2)cc1 ; c1ccc(N(c2ccc3ccc4ccccc4c3c2)c2ccccc2-c2ccc(C3(c4ccccc4)c4ccccc4-c4ccccc43)cc2)cc1]

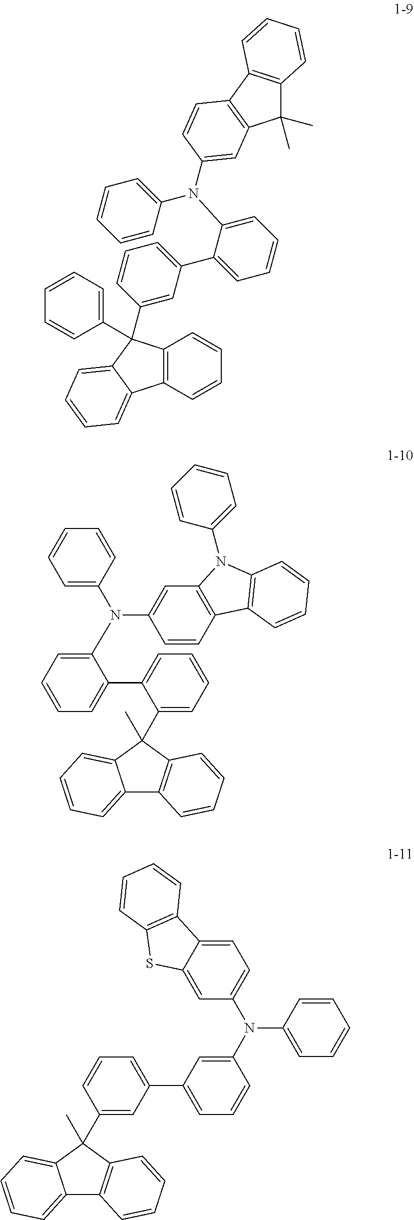 [SMILES: CC1(C)c2ccccc2-c2ccc(N(c3ccccc3)c3ccccc3-c3cccc(C4(c5ccccc5)c5ccccc5-c5ccccc54)c3)cc21 ; CC1(c2ccccc2-c2ccccc2N(c2ccccc2)c2ccc3c4ccccc4n(-c4ccccc4)c3c2)c2ccccc2-c2ccccc21 ; CC1(c2cccc(-c3cccc(N(c4ccccc4)c4ccc5c(c4)sc4ccccc45)c3)c2)c2ccccc2-c2ccccc21]

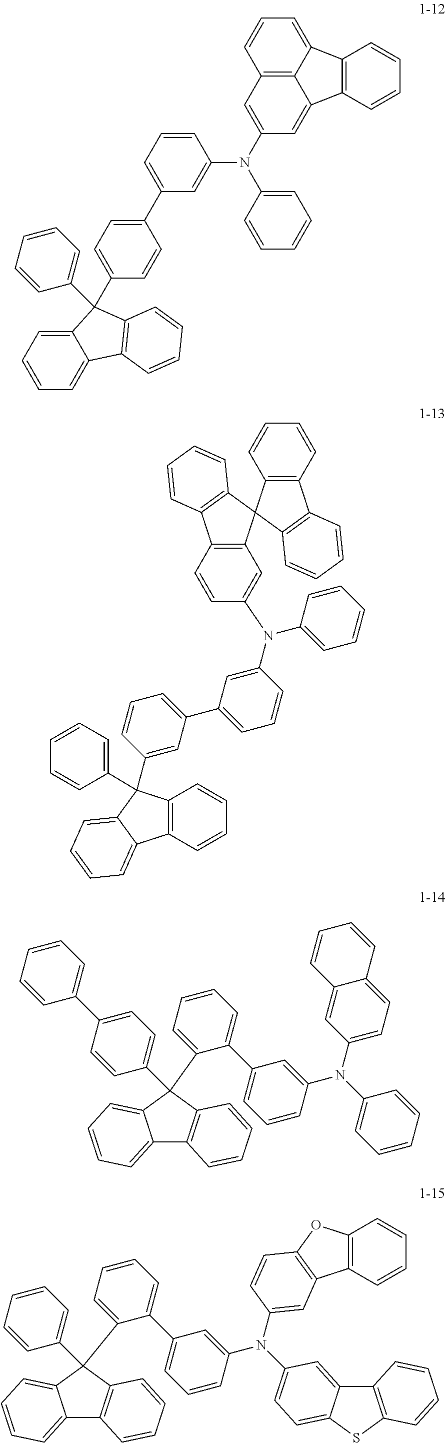 [SMILES: c1ccc(N(c2cccc(-c3ccc(C4(c5ccccc5)c5ccccc5-c5ccccc54)cc3)c2)c2cc3c4c(cccc4c2)-c2ccccc2-3)cc1 ; c1ccc(N(c2cccc(-c3cccc(C4(c5ccccc5)c5ccccc5-c5ccccc54)c3)c2)c2ccc3c(c2)C2(c4ccccc4-c4ccccc42)c2ccccc2-3)cc1 ; c1ccc(-c2ccc(C3(c4ccccc4-c4cccc(N(c5ccccc5)c5ccc6ccccc6c5)c4)c4ccccc4-c4ccccc43)cc2)cc1 ; c1ccc(C2(c3ccccc3-c3cccc(N(c4ccc5oc6ccccc6c5c4)c4ccc5sc6ccccc6c5c4)c3)c3ccccc3-c3ccccc32)cc1]

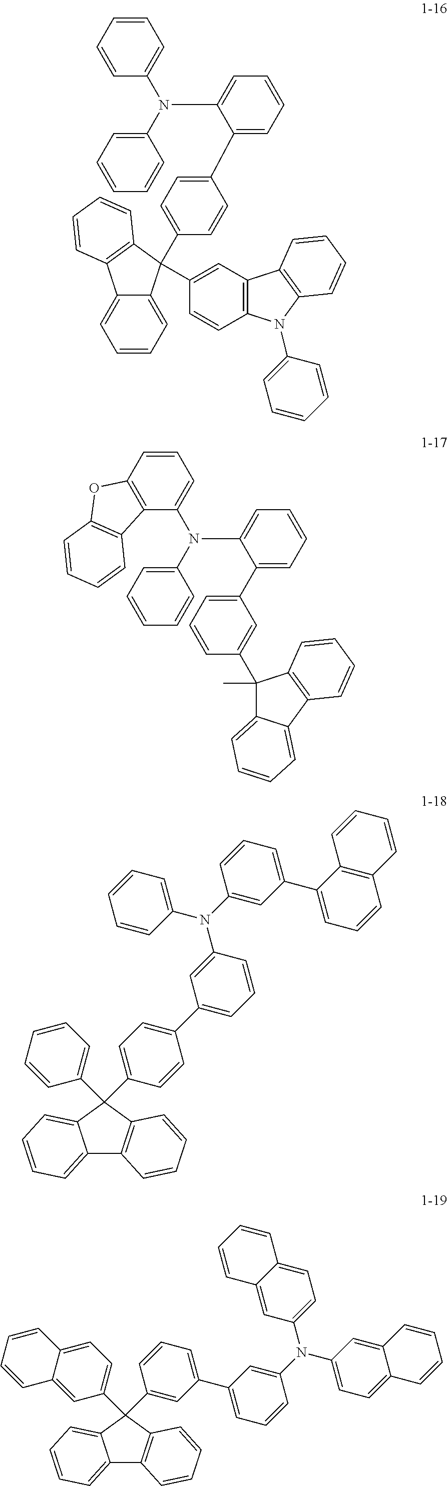 [SMILES: c1ccc(N(c2ccccc2)c2ccccc2-c2ccc(C3(c4ccc5c(c4)c4ccccc4n5-c4ccccc4)c4ccccc4-c4ccccc43)cc2)cc1 ; CC1(c2cccc(-c3ccccc3N(c3ccccc3)c3cccc4oc5ccccc5c34)c2)c2ccccc2-c2ccccc21 ; c1ccc(N(c2cccc(-c3ccc(C4(c5ccccc5)c5ccccc5-c5ccccc54)cc3)c2)c2cccc(-c3cccc4ccccc34)c2)cc1 ; c1cc(-c2cccc(C3(c4ccc5ccccc5c4)c4ccccc4-c4ccccc43)c2)cc(N(c2ccc3ccccc3c2)c2ccc3ccccc3c2)c1]

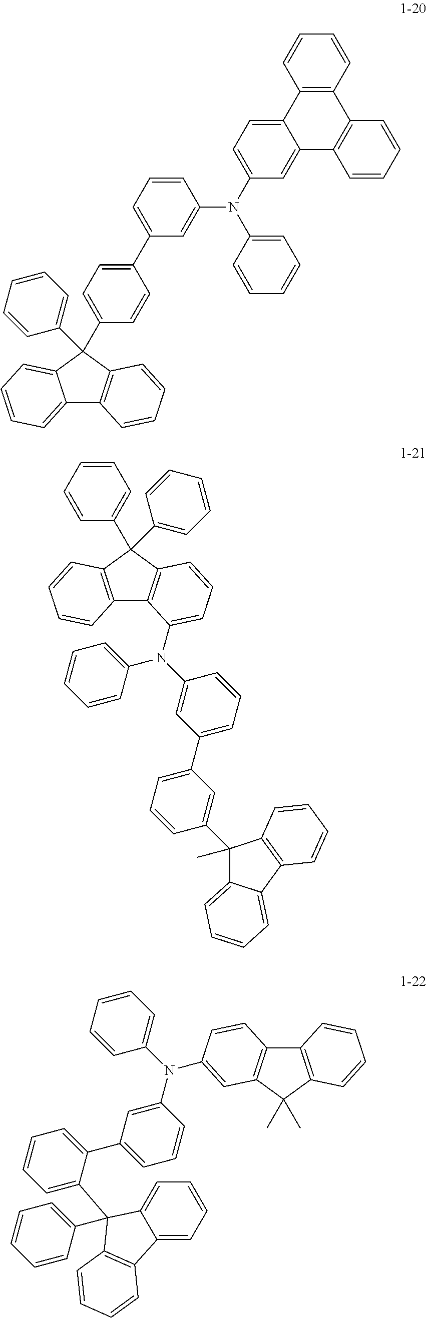

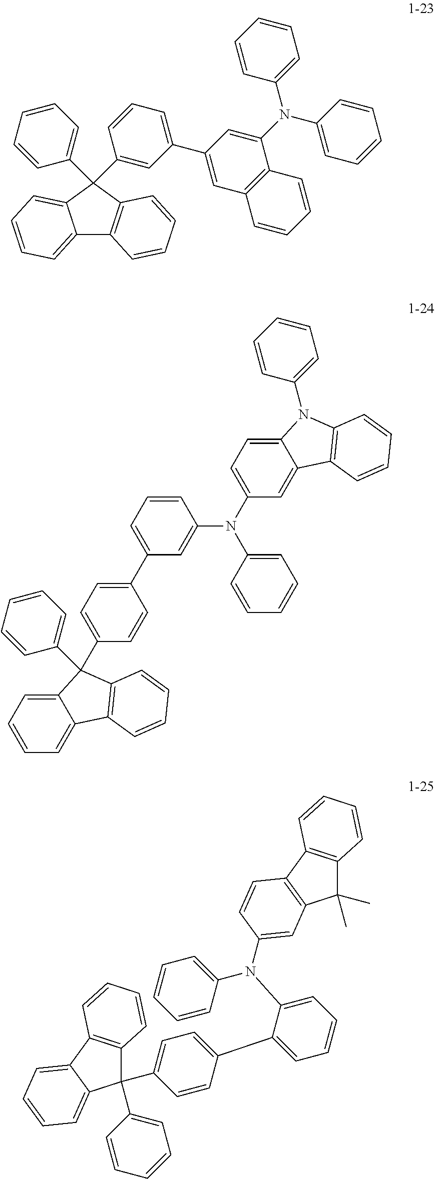 [SMILES: c1ccc(N(c2ccccc2)c2cc(-c3cccc(C4(c5ccccc5)c5ccccc5-c5ccccc54)c3)cc3ccccc23)cc1 ; c1ccc(N(c2cccc(-c3ccc(C4(c5ccccc5)c5ccccc5-c5ccccc54)cc3)c2)c2ccc3c(c2)c2ccccc2n3-c2ccccc2)cc1 ; CC1(C)c2ccccc2-c2ccc(N(c3ccccc3)c3ccccc3-c3ccc(C4(c5ccccc5)c5ccccc5-c5ccccc54)cc3)cc21]

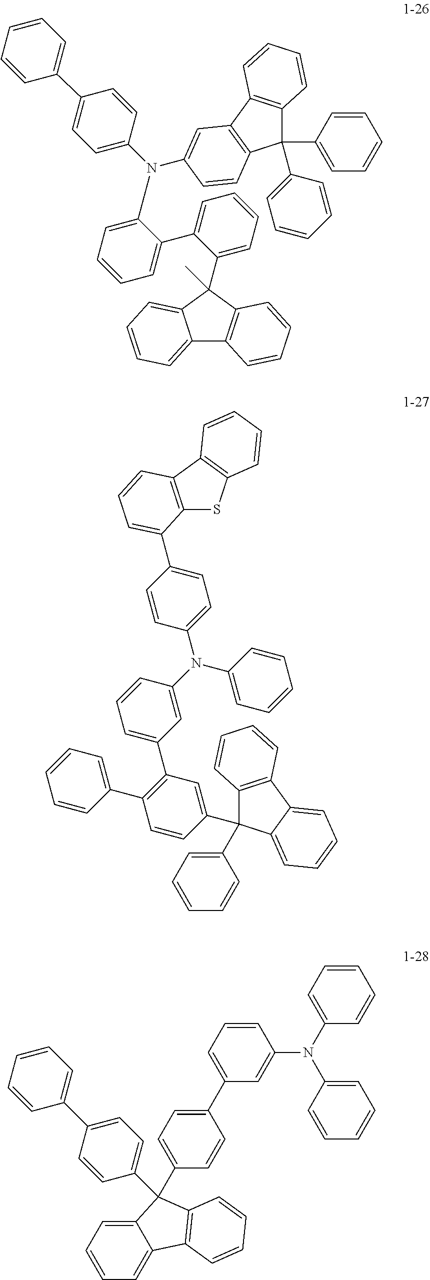 [SMILES: CC1(c2ccccc2-c2ccccc2N(c2ccc(-c3ccccc3)cc2)c2ccc3c(c2)-c2ccccc2C3(c2ccccc2)c2ccccc2)c2ccccc2-c2ccccc21 ; c1ccc(-c2ccc(C3(c4ccccc4)c4ccccc4-c4ccccc43)cc2-c2cccc(N(c3ccccc3)c3ccc(-c4cccc5c4sc4ccccc45)cc3)c2)cc1 ; c1ccc(-c2ccc(C3(c4ccc(-c5cccc(N(c6ccccc6)c6ccccc6)c5)cc4)c4ccccc4-c4ccccc43)cc2)cc1]

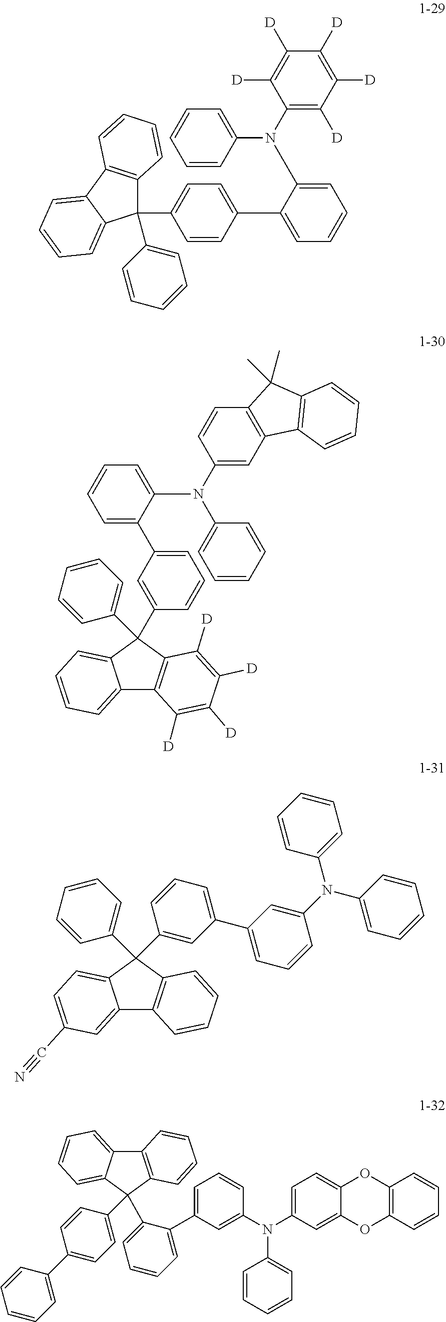 [SMILES: [2H]c1c([2H])c([2H])c(N(c2ccccc2)c2ccccc2-c2ccc(C3(c4ccccc4)c4ccccc4-c4ccccc43)cc2)c([2H])c1[2H] ; [2H]c1c([2H])c([2H])c2c(c1[2H])-c1ccccc1C2(c1ccccc1)c1cccc(-c2ccccc2N(c2ccccc2)c2ccc3c(c2)-c2ccccc2C3(C)C)c1 ; N#Cc1ccc2c(c1)-c1ccccc1C2(c1ccccc1)c1cccc(-c2cccc(N(c3ccccc3)c3ccccc3)c2)c1 ; c1ccc(-c2ccc(C3(c4ccccc4-c4cccc(N(c5ccccc5)c5ccc6c(c5)Oc5ccccc5O6)c4)c4ccccc4-c4ccccc43)cc2)cc1]

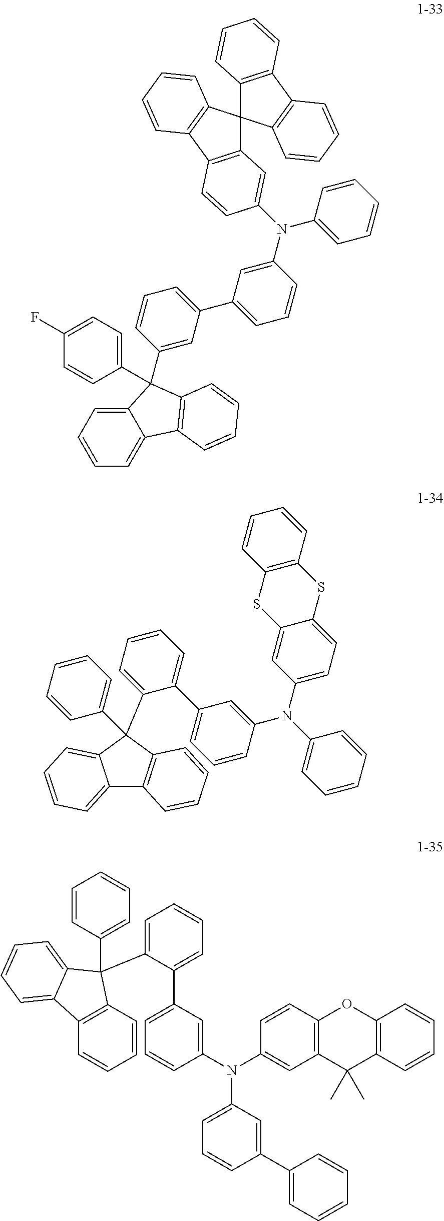 [SMILES: Fc1ccc(C2(c3cccc(-c4cccc(N(c5ccccc5)c5ccc6c(c5)C5(c7ccccc7-c7ccccc75)c5ccccc5-6)c4)c3)c3ccccc3-c3ccccc32)cc1 ; c1ccc(N(c2cccc(-c3ccccc3C3(c4ccccc4)c4ccccc4-c4ccccc43)c2)c2ccc3c(c2)Sc2ccccc2S3)cc1 ; CC1(C)c2ccccc2Oc2ccc(N(c3cccc(-c4ccccc4)c3)c3cccc(-c4ccccc4C4(c5ccccc5)c5ccccc5-c5ccccc54)c3)cc21]

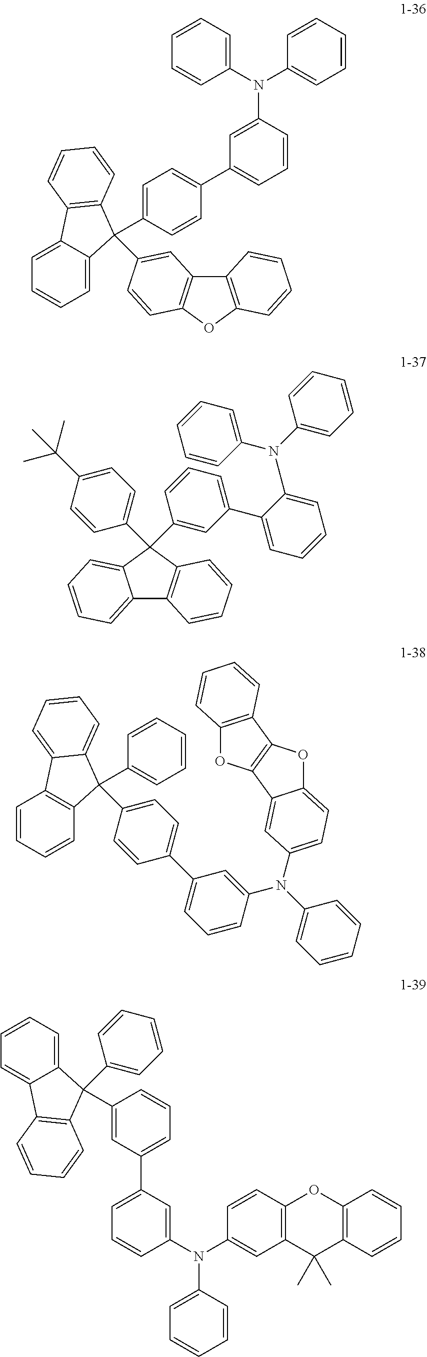 [SMILES: c1ccc(N(c2ccccc2)c2cccc(-c3ccc(C4(c5ccc6oc7ccccc7c6c5)c5ccccc5-c5ccccc54)cc3)c2)cc1 ; CC(C)(C)c1ccc(C2(c3cccc(-c4ccccc4N(c4ccccc4)c4ccccc4)c3)c3ccccc3-c3ccccc32)cc1 ; c1ccc(N(c2cccc(-c3ccc(C4(c5ccccc5)c5ccccc5-c5ccccc54)cc3)c2)c2ccc3oc4c5ccccc5oc4c3c2)cc1 ; CC1(C)c2ccccc2Oc2ccc(N(c3ccccc3)c3cccc(-c4cccc(C5(c6ccccc6)c6ccccc6-c6ccccc65)c4)c3)cc21]

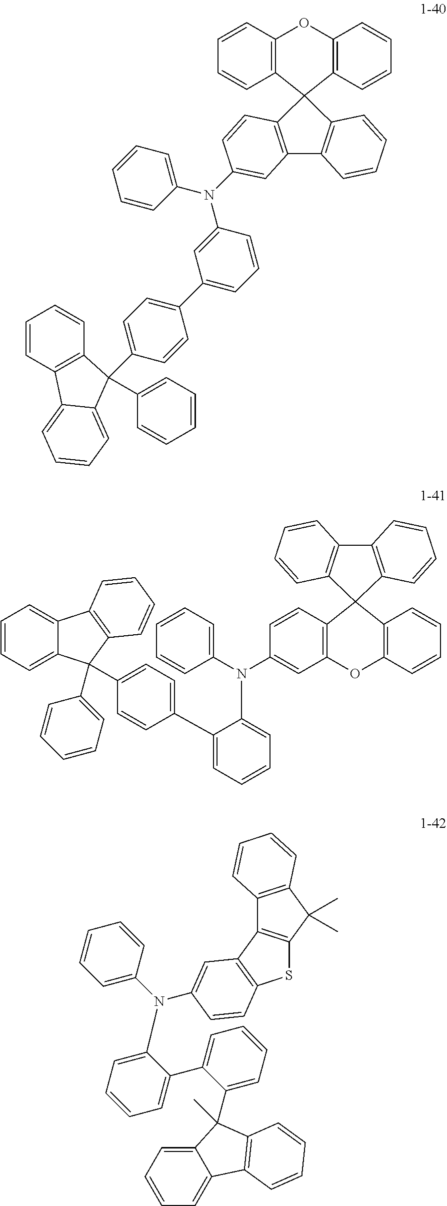 [SMILES: c1ccc(N(c2cccc(-c3ccc(C4(c5ccccc5)c5ccccc5-c5ccccc54)cc3)c2)c2ccc3c(c2)-c2ccccc2C32c3ccccc3Oc3ccccc32)cc1 ; c1ccc(N(c2ccc3c(c2)Oc2ccccc2C32c3ccccc3-c3ccccc32)c2ccccc2-c2ccc(C3(c4ccccc4)c4ccccc4-c4ccccc43)cc2)cc1 ; CC1(C)c2ccccc2-c2c1sc1ccc(N(c3ccccc3)c3ccccc3-c3ccccc3C3(C)c4ccccc4-c4ccccc43)cc21]

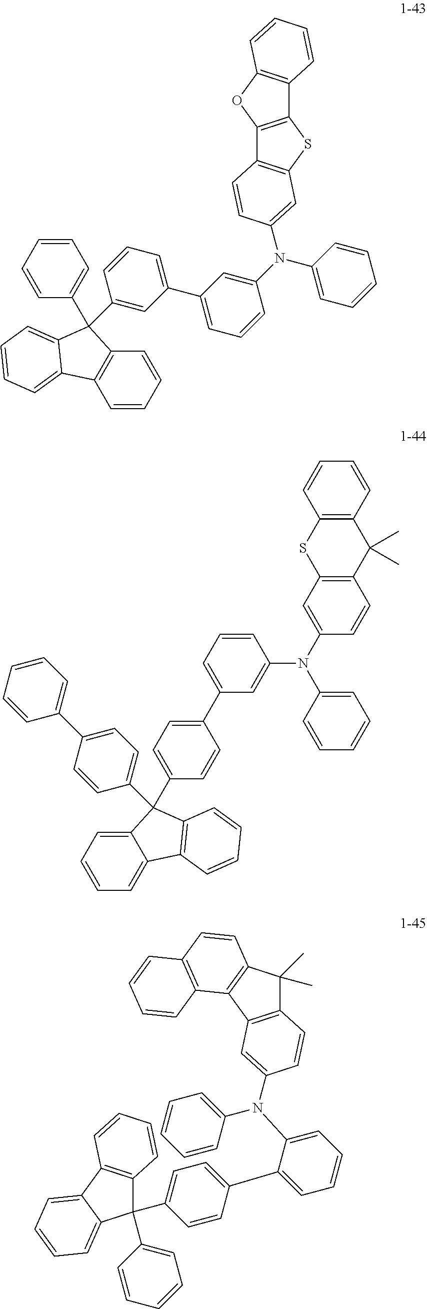 [SMILES: c1ccc(N(c2cccc(-c3cccc(C4(c5ccccc5)c5ccccc5-c5ccccc54)c3)c2)c2ccc3c(c2)sc2c4ccccc4oc32)cc1 ; CC1(C)c2ccccc2Sc2cc(N(c3ccccc3)c3cccc(-c4ccc(C5(c6ccc(-c7ccccc7)cc6)c6ccccc6-c6ccccc65)cc4)c3)ccc21 ; CC1(C)c2ccc(N(c3ccccc3)c3ccccc3-c3ccc(C4(c5ccccc5)c5ccccc5-c5ccccc54)cc3)cc2-c2c1ccc1ccccc21]

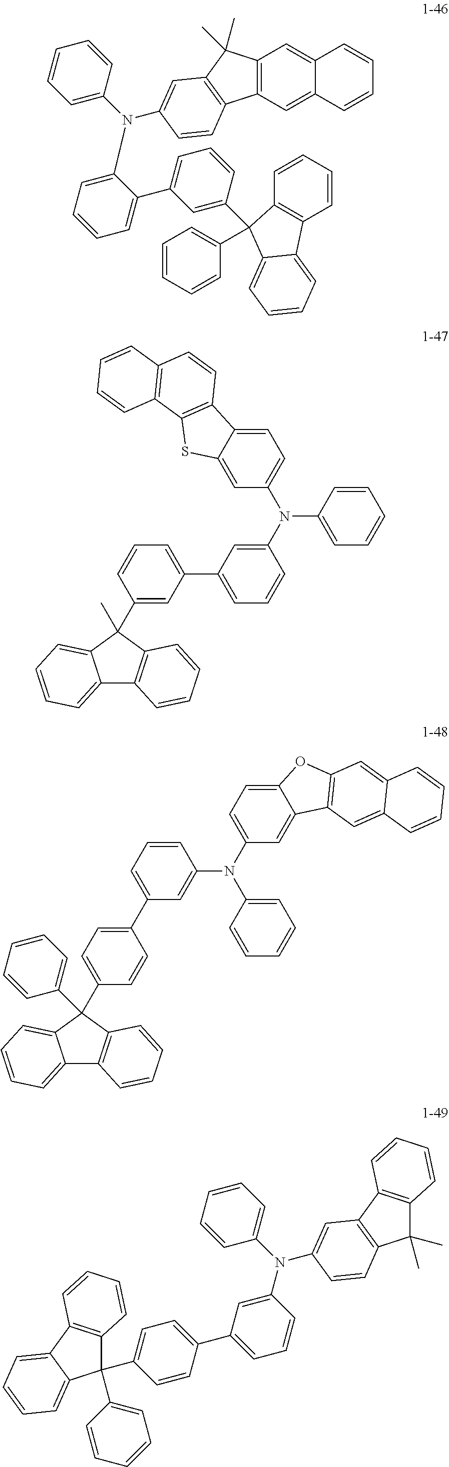 [SMILES: CC1(C)c2cc(N(c3ccccc3)c3ccccc3-c3cccc(C4(c5ccccc5)c5ccccc5-c5ccccc54)c3)ccc2-c2cc3ccccc3cc21 ; CC1(c2cccc(-c3cccc(N(c4ccccc4)c4ccc5c(c4)sc4c6ccccc6ccc54)c3)c2)c2ccccc2-c2ccccc21 ; c1ccc(N(c2cccc(-c3ccc(C4(c5ccccc5)c5ccccc5-c5ccccc54)cc3)c2)c2ccc3oc4cc5ccccc5cc4c3c2)cc1 ; CC1(C)c2ccccc2-c2cc(N(c3ccccc3)c3cccc(-c4ccc(C5(c6ccccc6)c6ccccc6-c6ccccc65)cc4)c3)ccc21]

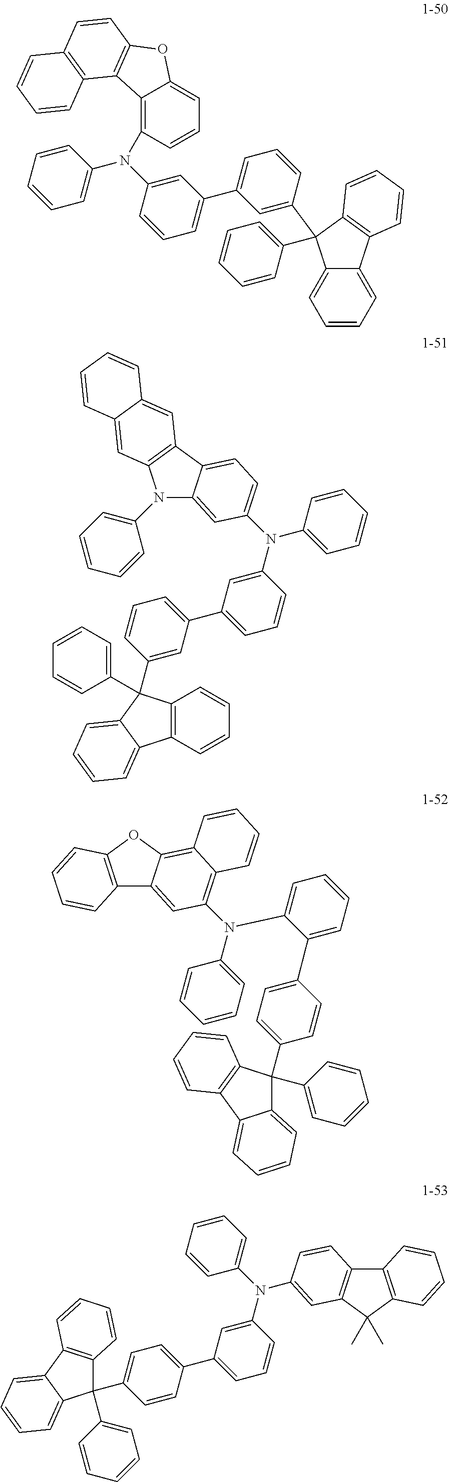 [SMILES: c1ccc(N(c2cccc(-c3cccc(C4(c5ccccc5)c5ccccc5-c5ccccc54)c3)c2)c2cccc3oc4ccc5ccccc5c4c23)cc1 ; c1ccc(N(c2cccc(-c3cccc(C4(c5ccccc5)c5ccccc5-c5ccccc54)c3)c2)c2ccc3c4cc5ccccc5cc4n(-c4ccccc4)c3c2)cc1 ; c1ccc(N(c2ccccc2-c2ccc(C3(c4ccccc4)c4ccccc4-c4ccccc43)cc2)c2cc3c4ccccc4oc3c3ccccc23)cc1 ; CC1(C)c2ccccc2-c2ccc(N(c3ccccc3)c3cccc(-c4ccc(C5(c6ccccc6)c6ccccc6-c6ccccc65)cc4)c3)cc21]

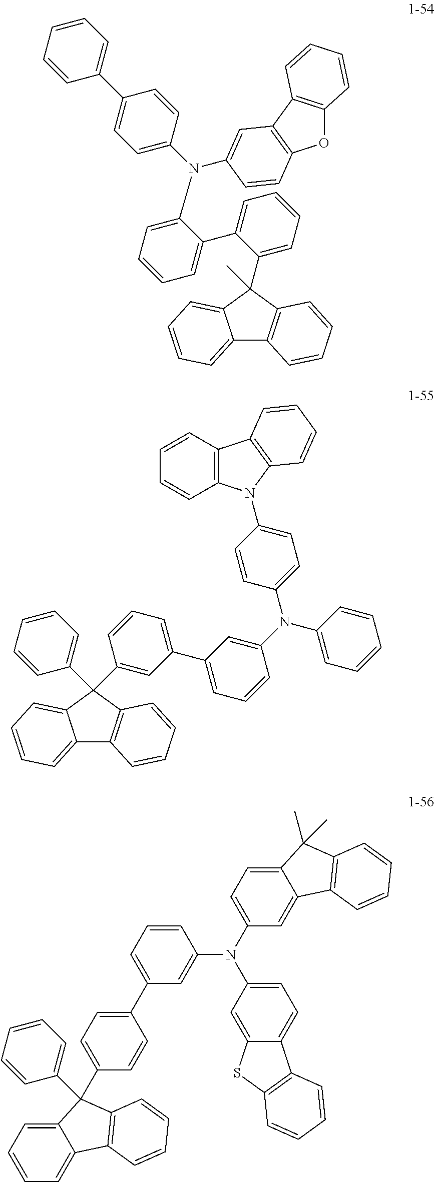

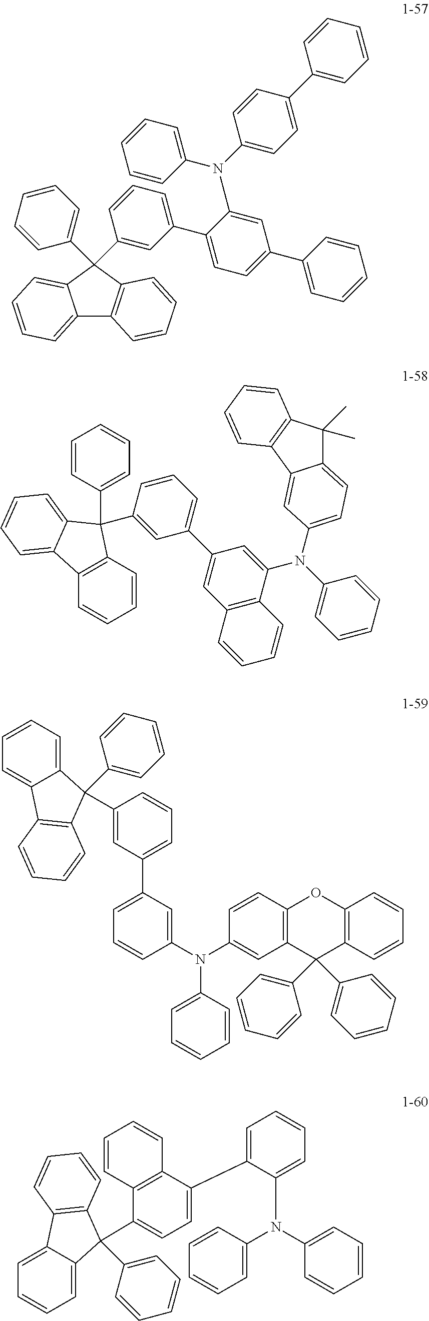 [SMILES: c1ccc(-c2ccc(N(c3ccccc3)c3cc(-c4ccccc4)ccc3-c3cccc(C4(c5ccccc5)c5ccccc5-c5ccccc54)c3)cc2)cc1 ; CC1(C)c2ccccc2-c2cc(N(c3ccccc3)c3cc(-c4cccc(C5(c6ccccc6)c6ccccc6-c6ccccc65)c4)cc4ccccc34)ccc21 ; c1ccc(N(c2cccc(-c3cccc(C4(c5ccccc5)c5ccccc5-c5ccccc54)c3)c2)c2ccc3c(c2)C(c2ccccc2)(c2ccccc2)c2ccccc2O3)cc1 ; c1ccc(N(c2ccccc2)c2ccccc2-c2ccc(C3(c4ccccc4)c4ccccc4-c4ccccc43)c3ccccc23)cc1]

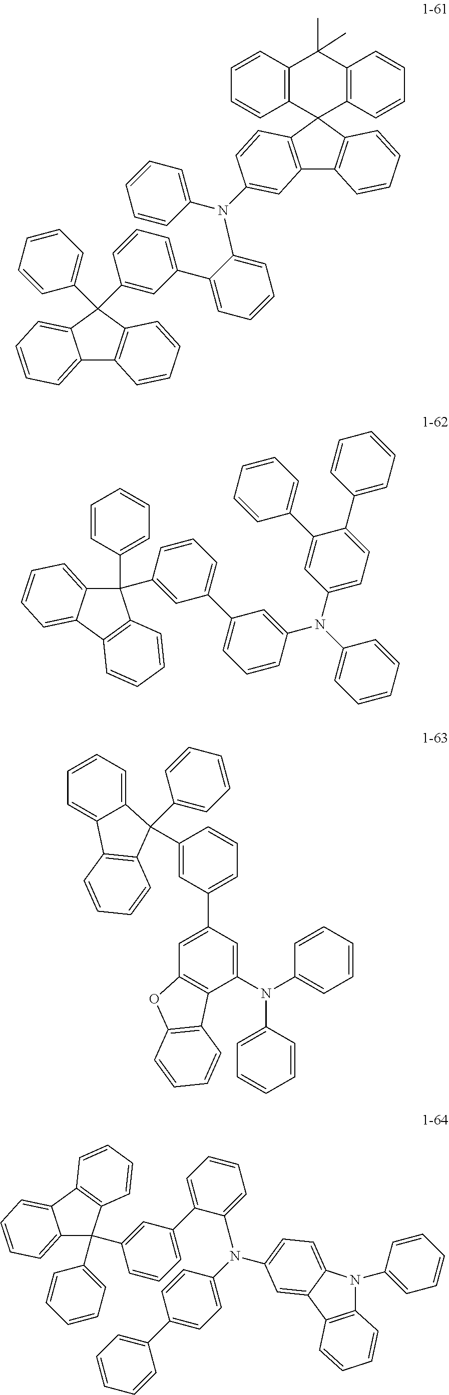 [SMILES: CC1(C)c2ccccc2C2(c3ccccc3-c3cc(N(c4ccccc4)c4ccccc4-c4cccc(C5(c6ccccc6)c6ccccc6-c6ccccc65)c4)ccc32)c2ccccc21 ; c1ccc(-c2ccc(N(c3ccccc3)c3cccc(-c4cccc(C5(c6ccccc6)c6ccccc6-c6ccccc65)c4)c3)cc2-c2ccccc2)cc1 ; c1ccc(N(c2ccccc2)c2cc(-c3cccc(C4(c5ccccc5)c5ccccc5-c5ccccc54)c3)cc3oc4ccccc4c23)cc1 ; c1ccc(-c2ccc(N(c3ccc4c(c3)c3ccccc3n4-c3ccccc3)c3ccccc3-c3cccc(C4(c5ccccc5)c5ccccc5-c5ccccc54)c3)cc2)cc1]

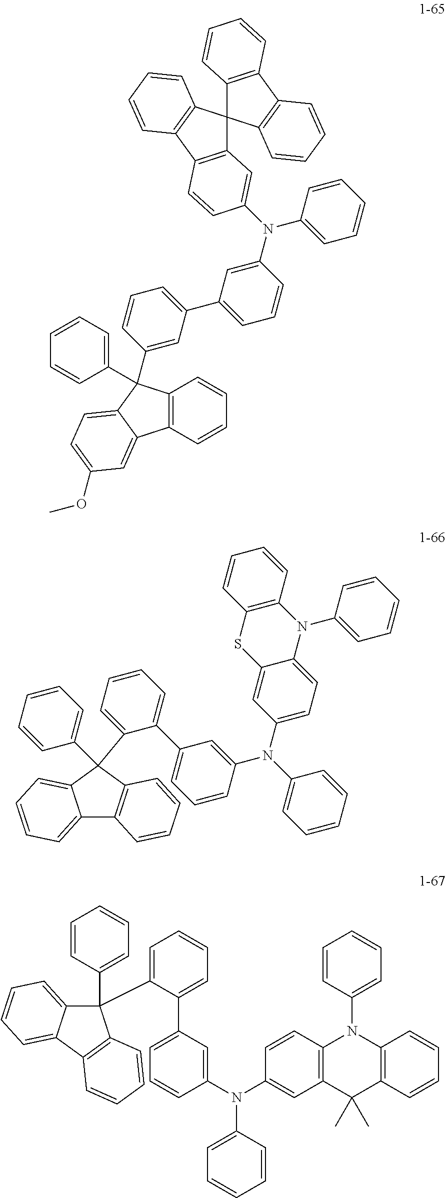 [SMILES: COc1ccc2c(c1)-c1ccccc1C2(c1ccccc1)c1cccc(-c2cccc(N(c3ccccc3)c3ccc4c(c3)C3(c5ccccc5-c5ccccc53)c3ccccc3-4)c2)c1 ; c1ccc(N(c2cccc(-c3ccccc3C3(c4ccccc4)c4ccccc4-c4ccccc43)c2)c2ccc3c(c2)Sc2ccccc2N3c2ccccc2)cc1 ; CC1(C)c2ccccc2N(c2ccccc2)c2ccc(N(c3ccccc3)c3cccc(-c4ccccc4C4(c5ccccc5)c5ccccc5-c5ccccc54)c3)cc21]

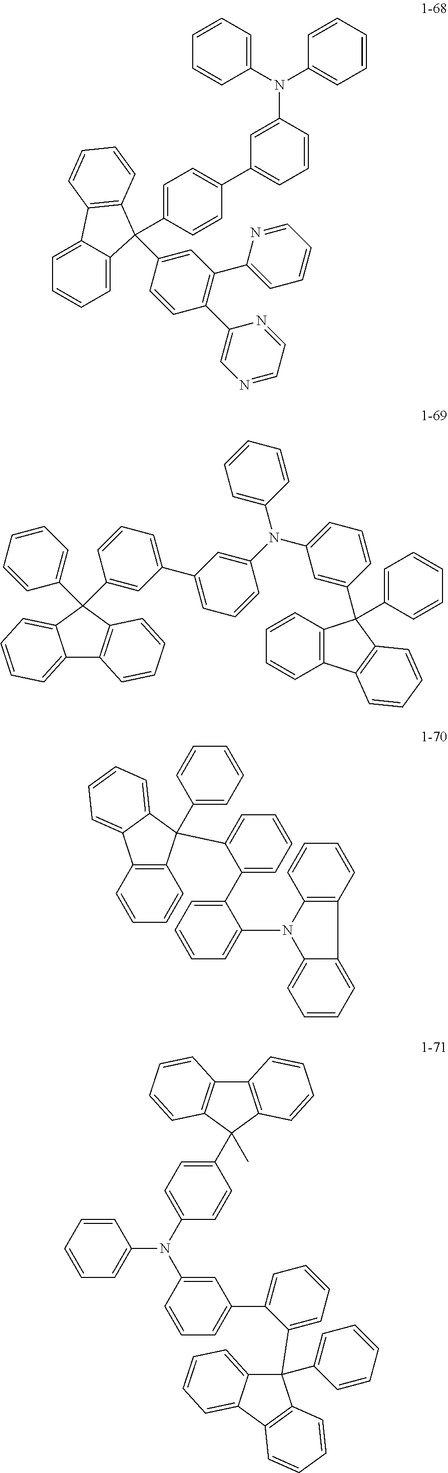 [SMILES: c1ccc(N(c2ccccc2)c2cccc(-c3ccc(C4(c5ccc(-c6cnccn6)c(-c6ccccn6)c5)c5ccccc5-c5ccccc54)cc3)c2)cc1 ; c1ccc(N(c2cccc(-c3cccc(C4(c5ccccc5)c5ccccc5-c5ccccc54)c3)c2)c2cccc(C3(c4ccccc4)c4ccccc4-c4ccccc43)c2)cc1 ; c1ccc(C2(c3ccccc3-c3ccccc3-n3c4ccccc4c4ccccc43)c3ccccc3-c3ccccc32)cc1 ; CC1(c2ccc(N(c3ccccc3)c3cccc(-c4ccccc4C4(c5ccccc5)c5ccccc5-c5ccccc54)c3)cc2)c2ccccc2-c2ccccc21]

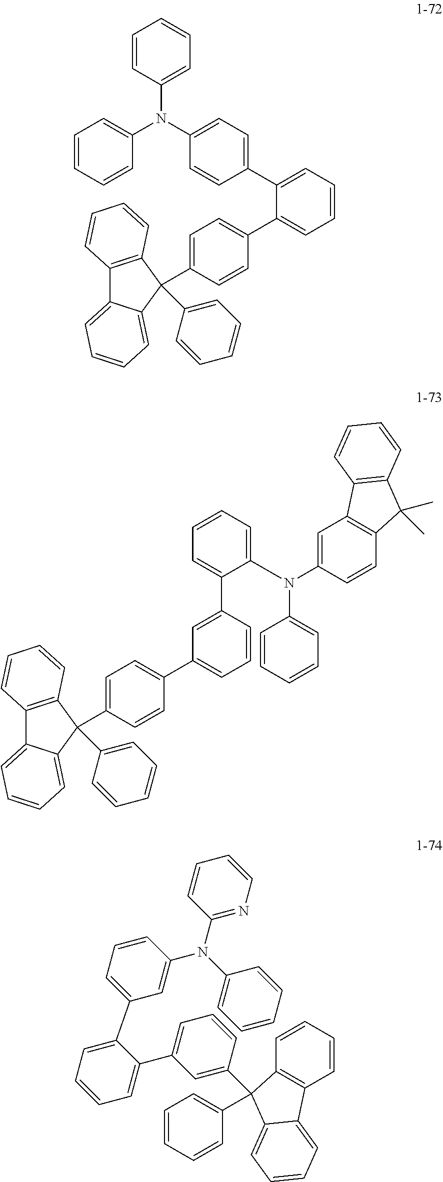 [SMILES: c1ccc(N(c2ccccc2)c2ccc(-c3ccccc3-c3ccc(C4(c5ccccc5)c5ccccc5-c5ccccc54)cc3)cc2)cc1 ; CC1(C)c2ccccc2-c2cc(N(c3ccccc3)c3ccccc3-c3cccc(-c4ccc(C5(c6ccccc6)c6ccccc6-c6ccccc65)cc4)c3)ccc21 ; c1ccc(N(c2cccc(-c3ccccc3-c3cccc(C4(c5ccccc5)c5ccccc5-c5ccccc54)c3)c2)c2ccccn2)cc1]

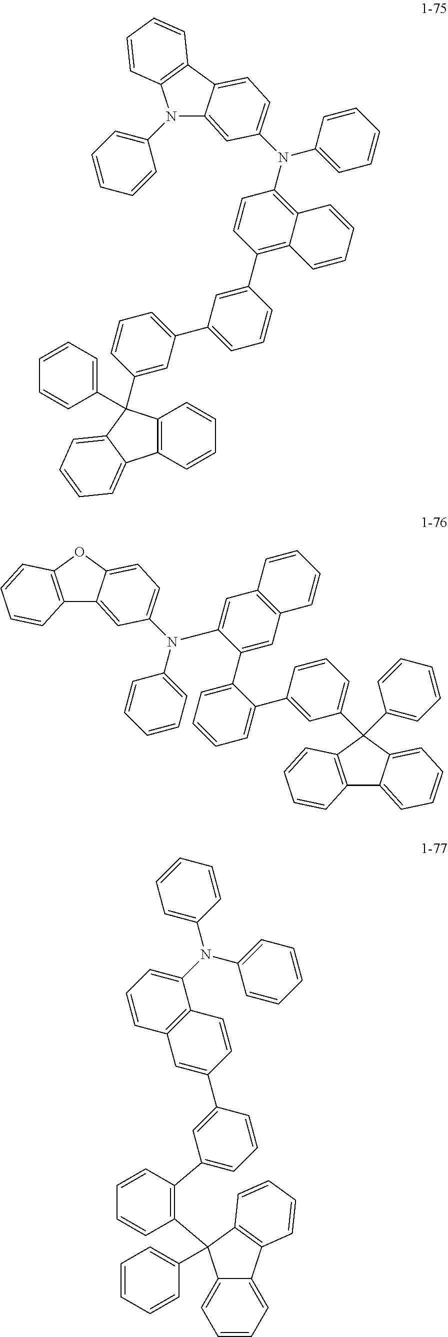 [SMILES: c1ccc(N(c2ccc3c4ccccc4n(-c4ccccc4)c3c2)c2ccc(-c3cccc(-c4cccc(C5(c6ccccc6)c6ccccc6-c6ccccc65)c4)c3)c3ccccc23)cc1 ; c1ccc(N(c2ccc3oc4ccccc4c3c2)c2cc3ccccc3cc2-c2ccccc2-c2cccc(C3(c4ccccc4)c4ccccc4-c4ccccc43)c2)cc1 ; c1ccc(N(c2ccccc2)c2cccc3cc(-c4cccc(-c5ccccc5C5(c6ccccc6)c6ccccc6-c6ccccc65)c4)ccc23)cc1]

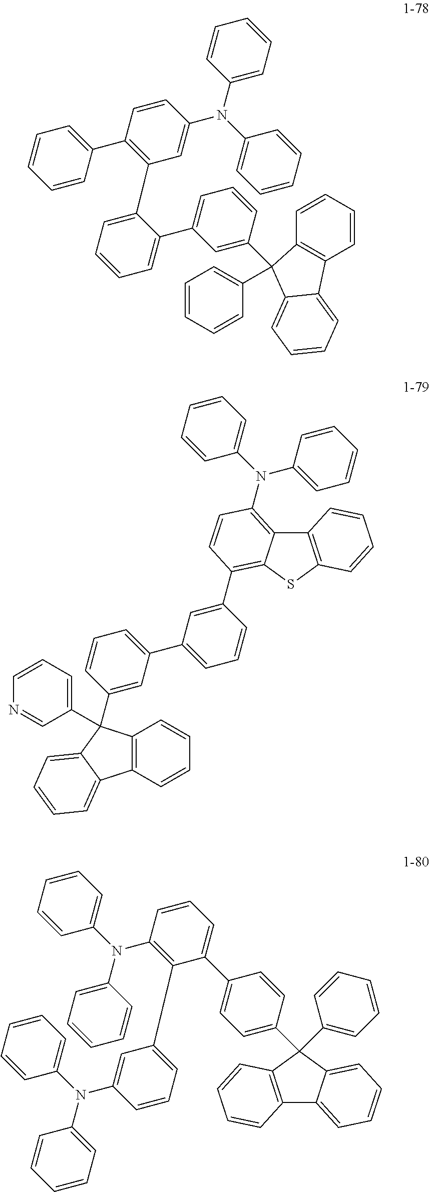 [SMILES: c1ccc(-c2ccc(N(c3ccccc3)c3ccccc3)cc2-c2ccccc2-c2cccc(C3(c4ccccc4)c4ccccc4-c4ccccc43)c2)cc1 ; c1ccc(N(c2ccccc2)c2ccc(-c3cccc(-c4cccc(C5(c6cccnc6)c6ccccc6-c6ccccc65)c4)c3)c3sc4ccccc4c23)cc1 ; c1ccc(N(c2ccccc2)c2cccc(-c3c(-c4ccc(C5(c6ccccc6)c6ccccc6-c6ccccc65)cc4)cccc3N(c3ccccc3)c3ccccc3)c2)cc1]

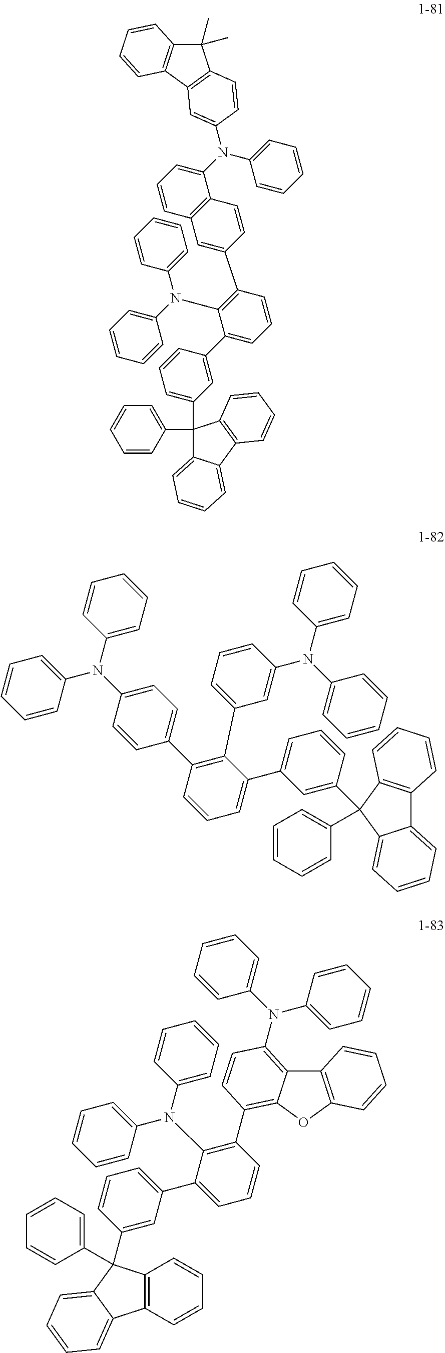 [SMILES: CC1(C)c2ccccc2-c2cc(N(c3ccccc3)c3cccc4cc(-c5cccc(-c6cccc(C7(c8ccccc8)c8ccccc8-c8ccccc87)c6)c5N(c5ccccc5)c5ccccc5)ccc34)ccc21 ; c1ccc(N(c2ccccc2)c2ccc(-c3cccc(-c4cccc(C5(c6ccccc6)c6ccccc6-c6ccccc65)c4)c3-c3cccc(N(c4ccccc4)c4ccccc4)c3)cc2)cc1 ; c1ccc(N(c2ccccc2)c2c(-c3cccc(C4(c5ccccc5)c5ccccc5-c5ccccc54)c3)cccc2-c2ccc(N(c3ccccc3)c3ccccc3)c3c2oc2ccccc23)cc1]

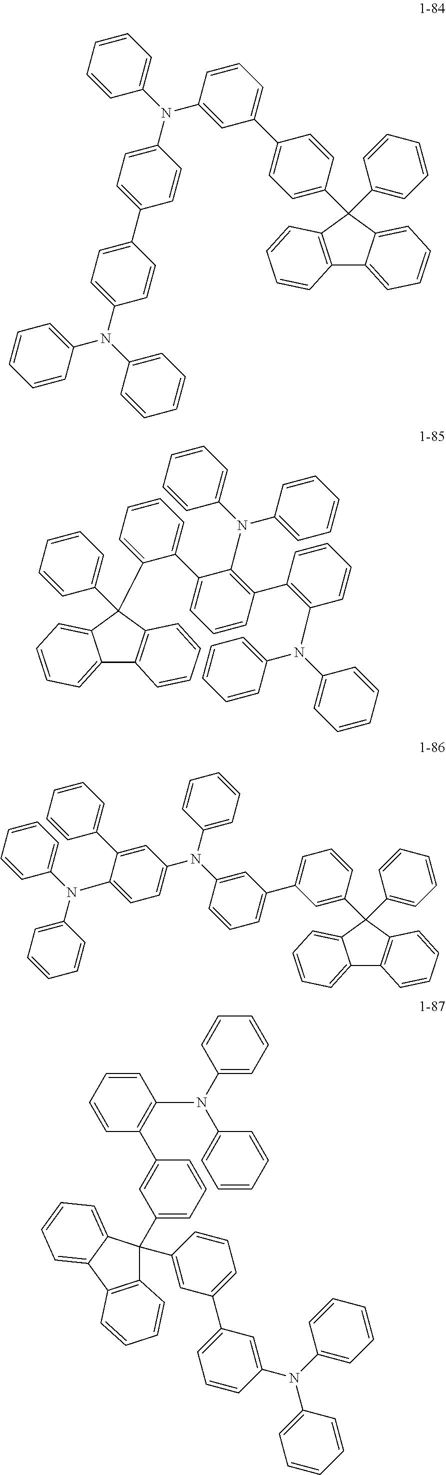

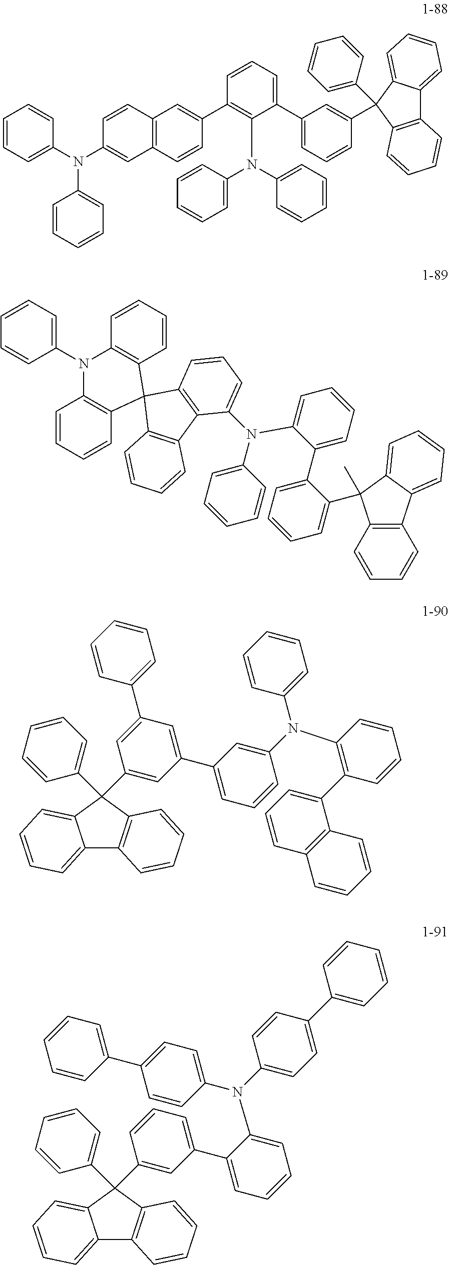 [SMILES: c1ccc(N(c2ccccc2)c2ccc3cc(-c4cccc(-c5cccc(C6(c7ccccc7)c7ccccc7-c7ccccc76)c5)c4N(c4ccccc4)c4ccccc4)ccc3c2)cc1 ; CC1(c2ccccc2-c2ccccc2N(c2ccccc2)c2cccc3c2-c2ccccc2C32c3ccccc3N(c3ccccc3)c3ccccc32)c2ccccc2-c2ccccc21 ; c1ccc(-c2cc(-c3cccc(N(c4ccccc4)c4ccccc4-c4cccc5ccccc45)c3)cc(C3(c4ccccc4)c4ccccc4-c4ccccc43)c2)cc1 ; c1ccc(-c2ccc(N(c3ccc(-c4ccccc4)cc3)c3ccccc3-c3cccc(C4(c5ccccc5)c5ccccc5-c5ccccc54)c3)cc2)cc1]

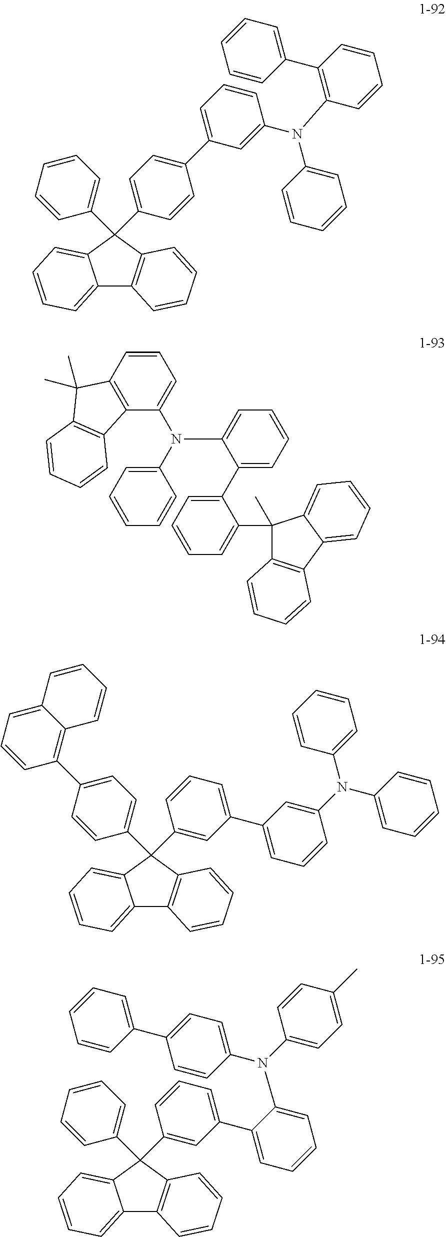

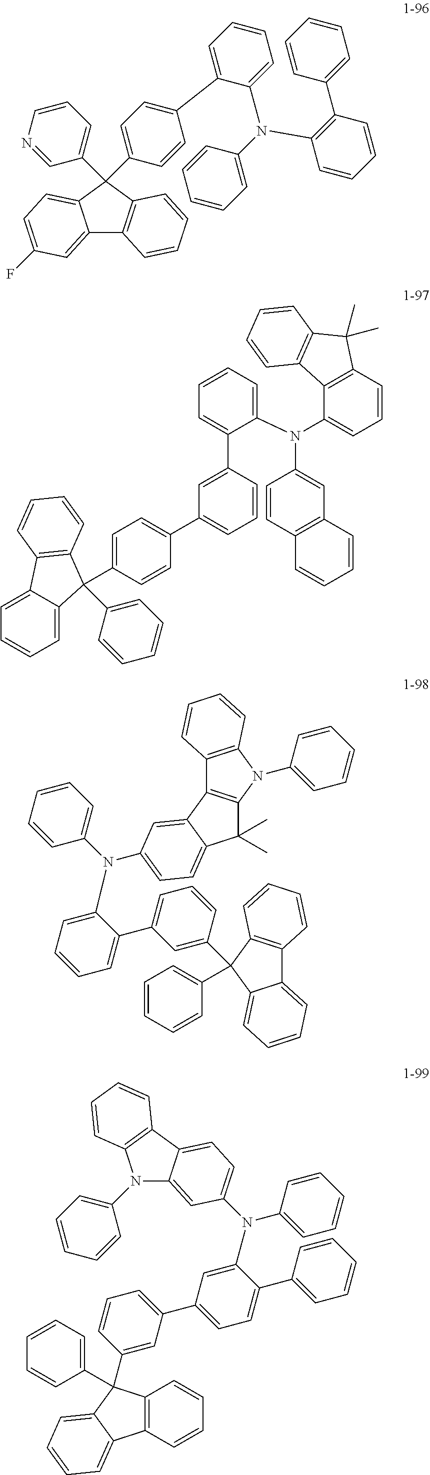 [SMILES: Fc1ccc2c(c1)-c1ccccc1C2(c1ccc(-c2ccccc2N(c2ccccc2)c2ccccc2-c2ccccc2)cc1)c1cccnc1 ; CC1(C)c2ccccc2-c2c(N(c3ccc4ccccc4c3)c3ccccc3-c3cccc(-c4ccc(C5(c6ccccc6)c6ccccc6-c6ccccc65)cc4)c3)cccc21 ; CC1(C)c2ccc(N(c3ccccc3)c3ccccc3-c3cccc(C4(c5ccccc5)c5ccccc5-c5ccccc54)c3)cc2-c2c1n(-c1ccccc1)c1ccccc21 ; c1ccc(-c2ccc(-c3cccc(C4(c5ccccc5)c5ccccc5-c5ccccc54)c3)cc2N(c2ccccc2)c2ccc3c4ccccc4n(-c4ccccc4)c3c2)cc1]

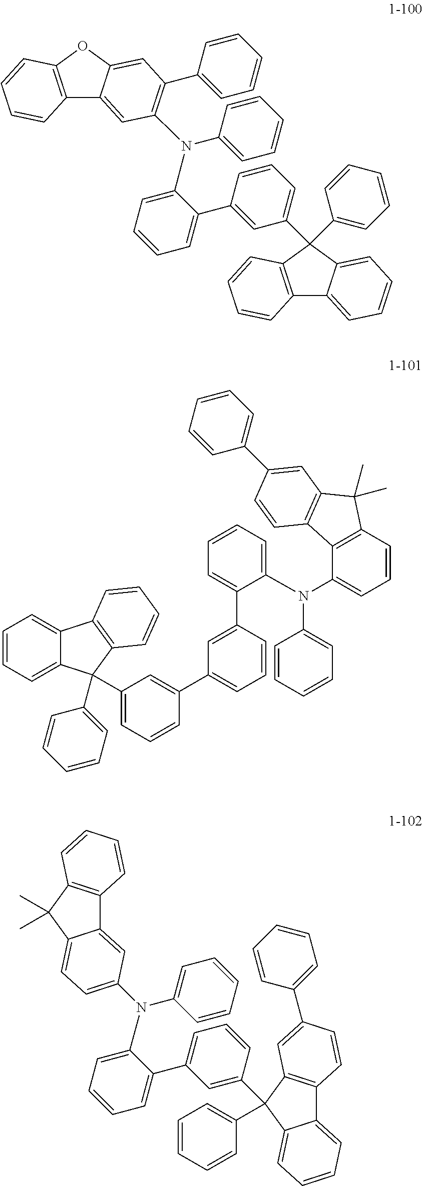 [SMILES: c1ccc(-c2cc3oc4ccccc4c3cc2N(c2ccccc2)c2ccccc2-c2cccc(C3(c4ccccc4)c4ccccc4-c4ccccc43)c2)cc1 ; CC1(C)c2cc(-c3ccccc3)ccc2-c2c(N(c3ccccc3)c3ccccc3-c3cccc(-c4cccc(C5(c6ccccc6)c6ccccc6-c6ccccc65)c4)c3)cccc21 ; CC1(C)c2ccccc2-c2cc(N(c3ccccc3)c3ccccc3-c3cccc(C4(c5ccccc5)c5ccccc5-c5ccc(-c6ccccc6)cc54)c3)ccc21]

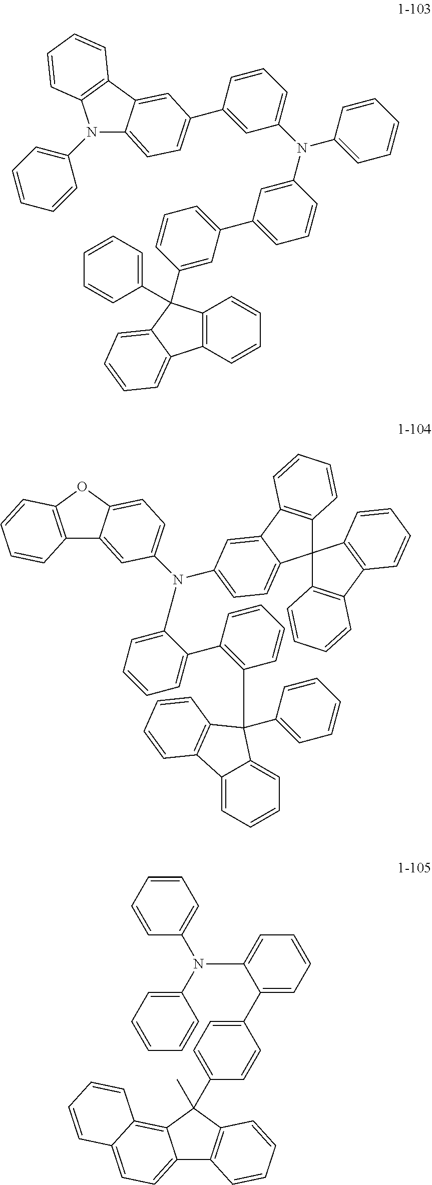 [SMILES: c1ccc(N(c2cccc(-c3cccc(C4(c5ccccc5)c5ccccc5-c5ccccc54)c3)c2)c2cccc(-c3ccc4c(c3)c3ccccc3n4-c3ccccc3)c2)cc1 ; c1ccc(C2(c3ccccc3-c3ccccc3N(c3ccc4c(c3)-c3ccccc3C43c4ccccc4-c4ccccc43)c3ccc4oc5ccccc5c4c3)c3ccccc3-c3ccccc32)cc1 ; CC1(c2ccc(-c3ccccc3N(c3ccccc3)c3ccccc3)cc2)c2ccccc2-c2ccc3ccccc3c21]

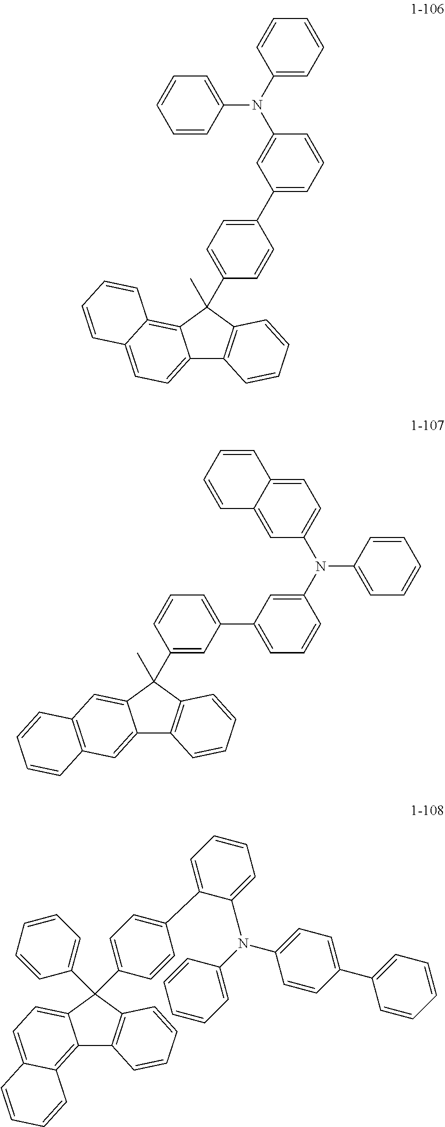 [SMILES: CC1(c2ccc(-c3cccc(N(c4ccccc4)c4ccccc4)c3)cc2)c2ccccc2-c2ccc3ccccc3c21 ; CC1(c2cccc(-c3cccc(N(c4ccccc4)c4ccc5ccccc5c4)c3)c2)c2ccccc2-c2cc3ccccc3cc21 ; c1ccc(-c2ccc(N(c3ccccc3)c3ccccc3-c3ccc(C4(c5ccccc5)c5ccccc5-c5c4ccc4ccccc54)cc3)cc2)cc1]

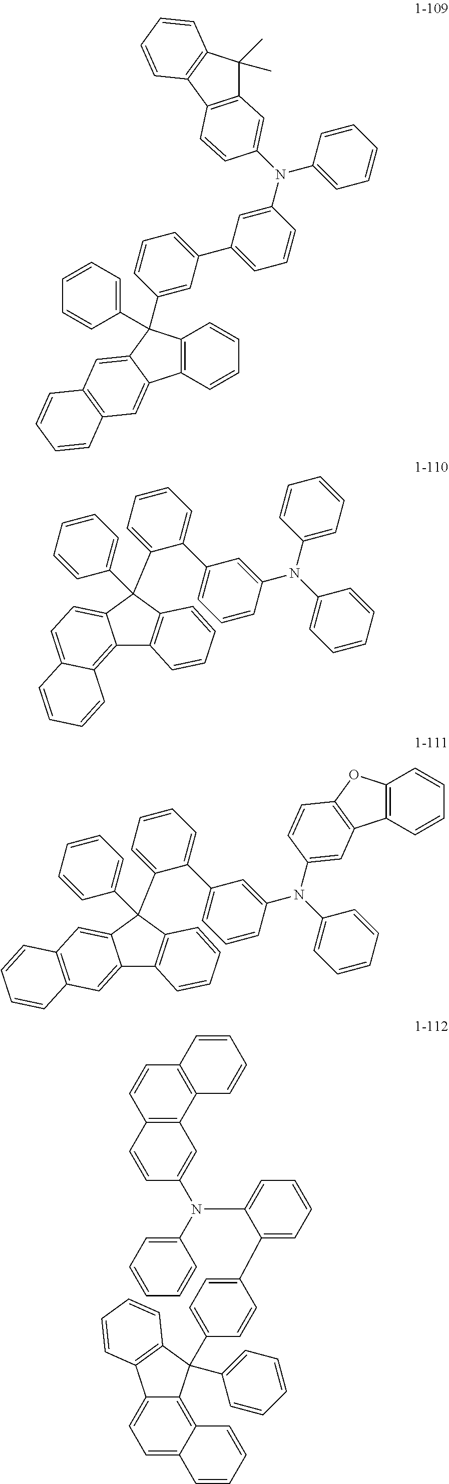 [SMILES: CC1(C)c2ccccc2-c2ccc(N(c3ccccc3)c3cccc(-c4cccc(C5(c6ccccc6)c6ccccc6-c6cc7ccccc7cc65)c4)c3)cc21 ; c1ccc(N(c2ccccc2)c2cccc(-c3ccccc3C3(c4ccccc4)c4ccccc4-c4c3ccc3ccccc43)c2)cc1 ; c1ccc(N(c2cccc(-c3ccccc3C3(c4ccccc4)c4ccccc4-c4cc5ccccc5cc43)c2)c2ccc3oc4ccccc4c3c2)cc1 ; c1ccc(N(c2ccc3ccc4ccccc4c3c2)c2ccccc2-c2ccc(C3(c4ccccc4)c4ccccc4-c4ccc5ccccc5c43)cc2)cc1]

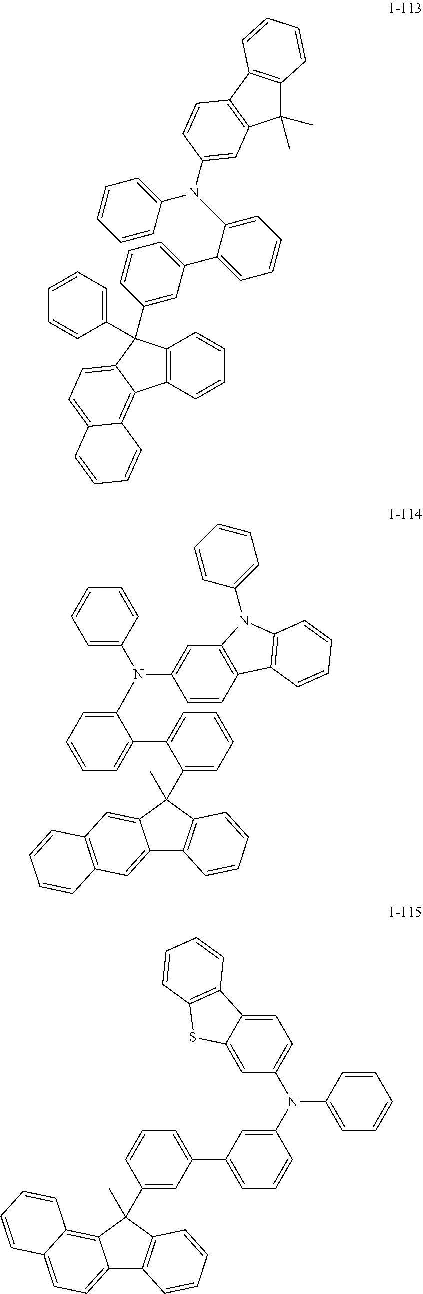 [SMILES: CC1(C)c2ccccc2-c2ccc(N(c3ccccc3)c3ccccc3-c3cccc(C4(c5ccccc5)c5ccccc5-c5c4ccc4ccccc54)c3)cc21 ; CC1(c2ccccc2-c2ccccc2N(c2ccccc2)c2ccc3c4ccccc4n(-c4ccccc4)c3c2)c2ccccc2-c2cc3ccccc3cc21 ; CC1(c2cccc(-c3cccc(N(c4ccccc4)c4ccc5c(c4)sc4ccccc45)c3)c2)c2ccccc2-c2ccc3ccccc3c21]

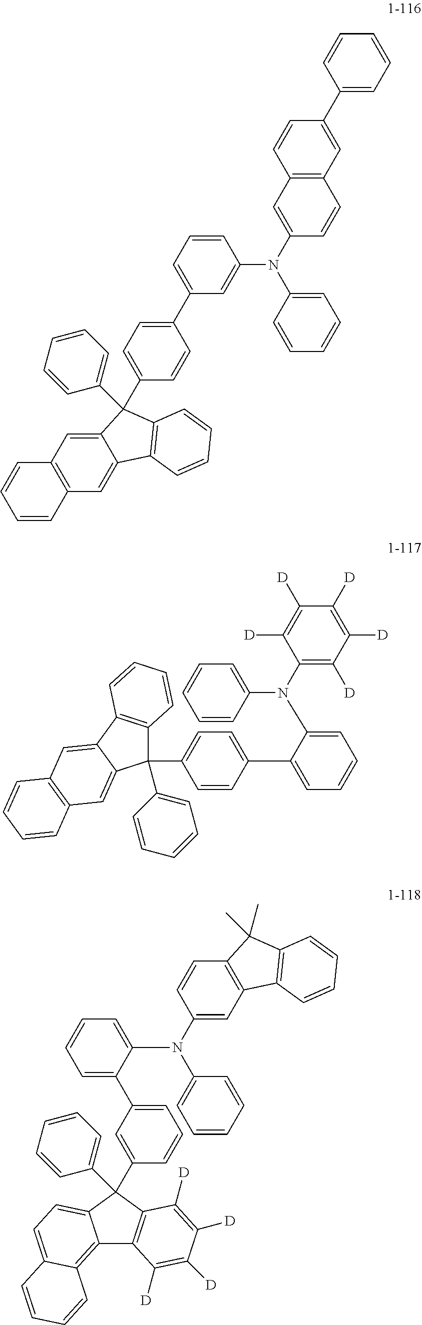 [SMILES: c1ccc(-c2ccc3cc(N(c4ccccc4)c4cccc(-c5ccc(C6(c7ccccc7)c7ccccc7-c7cc8ccccc8cc76)cc5)c4)ccc3c2)cc1 ; [2H]c1c([2H])c([2H])c(N(c2ccccc2)c2ccccc2-c2ccc(C3(c4ccccc4)c4ccccc4-c4cc5ccccc5cc43)cc2)c([2H])c1[2H] ; [2H]c1c([2H])c([2H])c2c(c1[2H])-c1c(ccc3ccccc13)C2(c1ccccc1)c1cccc(-c2ccccc2N(c2ccccc2)c2ccc3c(c2)-c2ccccc2C3(C)C)c1]

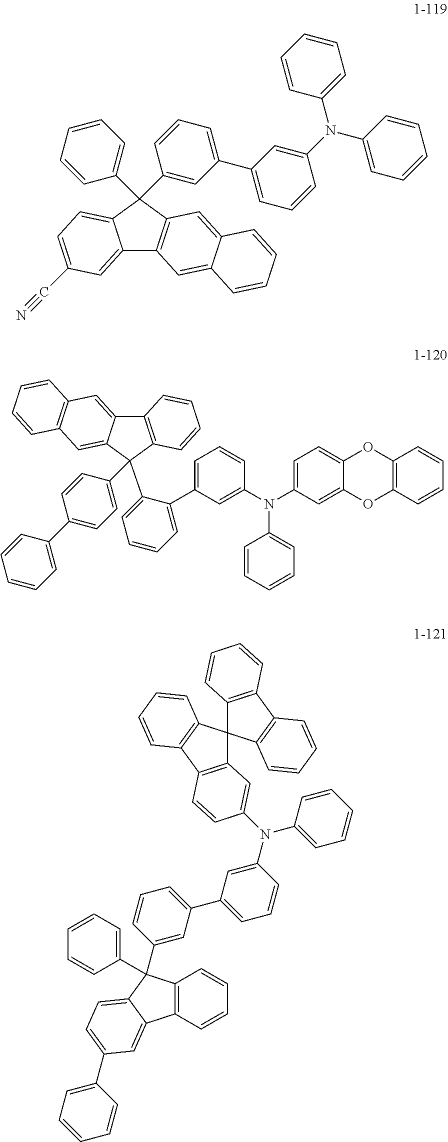 [SMILES: N#Cc1ccc2c(c1)-c1cc3ccccc3cc1C2(c1ccccc1)c1cccc(-c2cccc(N(c3ccccc3)c3ccccc3)c2)c1 ; c1ccc(-c2ccc(C3(c4ccccc4-c4cccc(N(c5ccccc5)c5ccc6c(c5)Oc5ccccc5O6)c4)c4ccccc4-c4cc5ccccc5cc43)cc2)cc1 ; c1ccc(-c2ccc3c(c2)-c2ccccc2C3(c2ccccc2)c2cccc(-c3cccc(N(c4ccccc4)c4ccc5c(c4)C4(c6ccccc6-c6ccccc64)c4ccccc4-5)c3)c2)cc1]

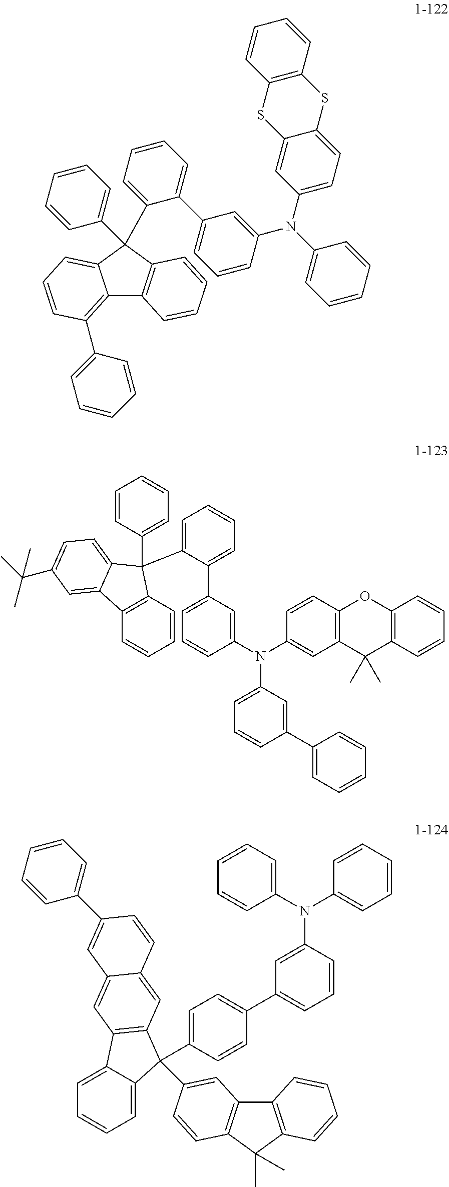 [SMILES: c1ccc(-c2cccc3c2-c2ccccc2C3(c2ccccc2)c2ccccc2-c2cccc(N(c3ccccc3)c3ccc4c(c3)Sc3ccccc3S4)c2)cc1 ; CC(C)(C)c1ccc2c(c1)-c1ccccc1C2(c1ccccc1)c1ccccc1-c1cccc(N(c2cccc(-c3ccccc3)c2)c2ccc3c(c2)C(C)(C)c2ccccc2O3)c1 ; CC1(C)c2ccccc2-c2cc(C3(c4ccc(-c5cccc(N(c6ccccc6)c6ccccc6)c5)cc4)c4ccccc4-c4cc5cc(-c6ccccc6)ccc5cc43)ccc21]

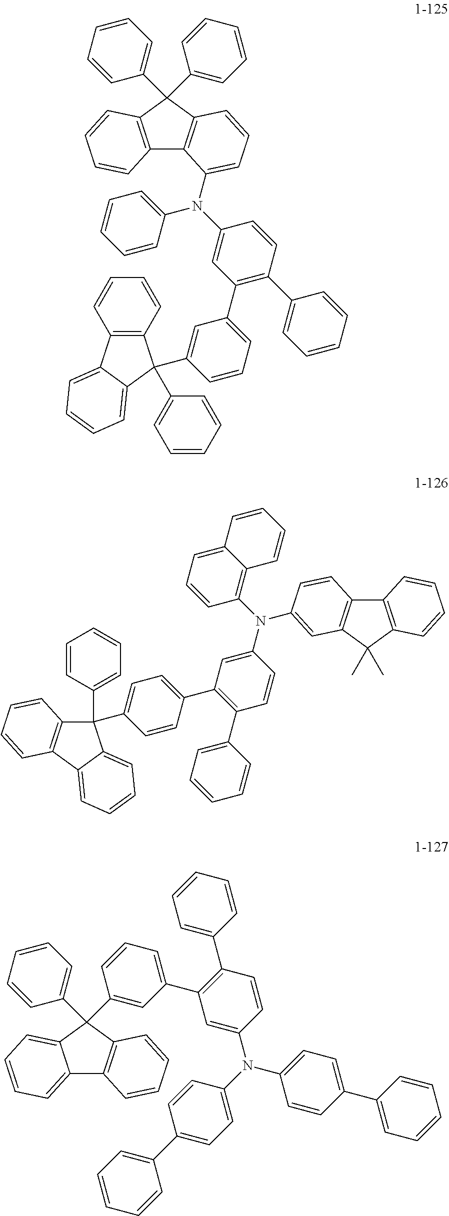 [SMILES: c1ccc(-c2ccc(N(c3ccccc3)c3cccc4c3-c3ccccc3C4(c3ccccc3)c3ccccc3)cc2-c2cccc(C3(c4ccccc4)c4ccccc4-c4ccccc43)c2)cc1 ; CC1(C)c2ccccc2-c2ccc(N(c3ccc(-c4ccccc4)c(-c4ccc(C5(c6ccccc6)c6ccccc6-c6ccccc65)cc4)c3)c3cccc4ccccc34)cc21 ; c1ccc(-c2ccc(N(c3ccc(-c4ccccc4)cc3)c3ccc(-c4ccccc4)c(-c4cccc(C5(c6ccccc6)c6ccccc6-c6ccccc65)c4)c3)cc2)cc1]

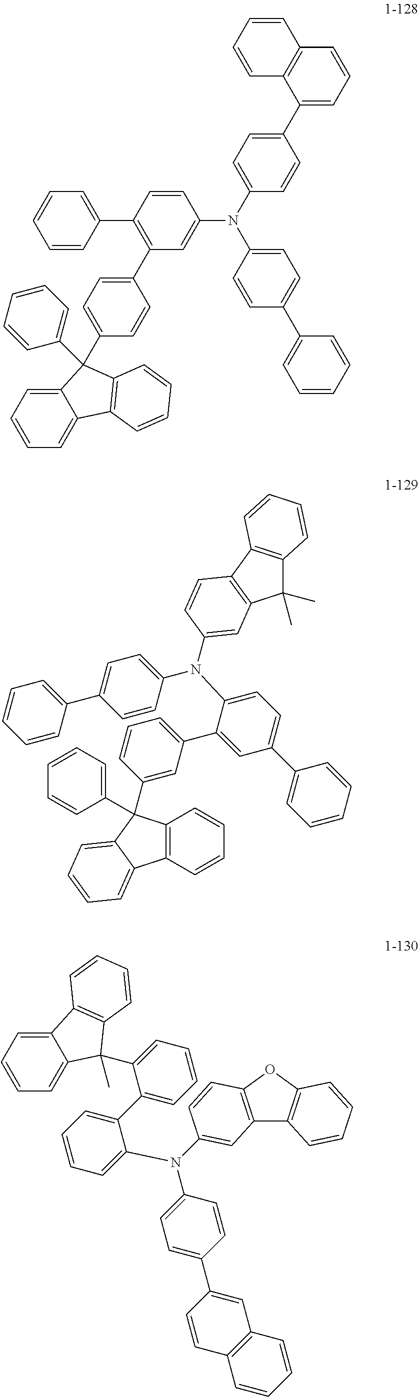 [SMILES: c1ccc(-c2ccc(N(c3ccc(-c4cccc5ccccc45)cc3)c3ccc(-c4ccccc4)c(-c4ccc(C5(c6ccccc6)c6ccccc6-c6ccccc65)cc4)c3)cc2)cc1 ; CC1(C)c2ccccc2-c2ccc(N(c3ccc(-c4ccccc4)cc3)c3ccc(-c4ccccc4)cc3-c3cccc(C4(c5ccccc5)c5ccccc5-c5ccccc54)c3)cc21 ; CC1(c2ccccc2-c2ccccc2N(c2ccc(-c3ccc4ccccc4c3)cc2)c2ccc3oc4ccccc4c3c2)c2ccccc2-c2ccccc21]

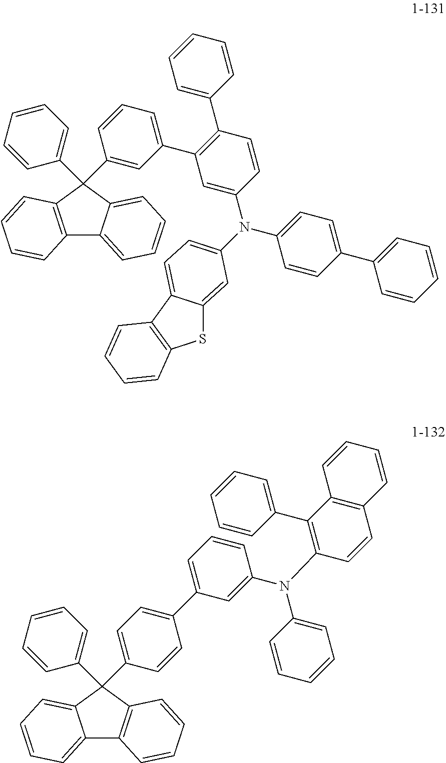 [SMILES: c1ccc(-c2ccc(N(c3ccc(-c4ccccc4)c(-c4cccc(C5(c6ccccc6)c6ccccc6-c6ccccc65)c4)c3)c3ccc4c(c3)sc3ccccc34)cc2)cc1 ; c1ccc(-c2c(N(c3ccccc3)c3cccc(-c4ccc(C5(c6ccccc6)c6ccccc6-c6ccccc65)cc4)c3)ccc3ccccc23)cc1]

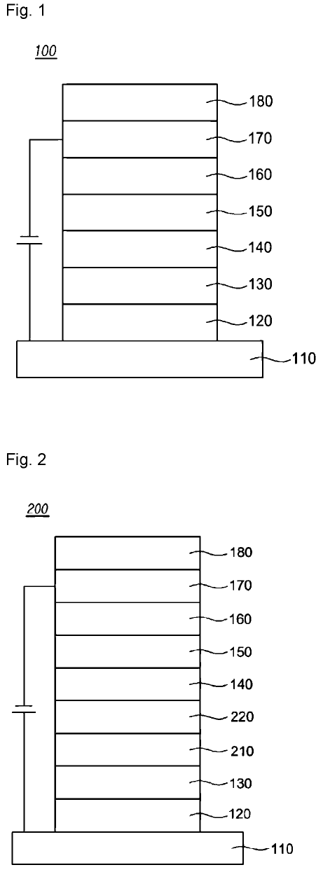

Referring to FIG. 1, the organic electronic element (100) according to the present invention includes a first electrode (110), a second electrode (170), and an organic material layer including a single compound or 2 or more compounds represented by Formula (1) between the first electrode (110) and the second electrode (170). In this case, the first electrode (110) may be an anode, and the second electrode (170) may be a cathode. In the case of an inverted type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer. The compound according to an embodiment of the present invention applied to the organic material layer may be used as a host or dopant of the hole injection layer (120), the hole transport layer (130), the emitting-auxiliary layer (220), electron transport auxiliary layer, the electron transport layer (150), and an electron injection layer (160), the emitting layer (140) or as a material for the light efficiency enhancing layer. Preferably, for example, the compound according to Formula (1) of the present invention may be used as a material for a hole transport layer, a host of the emitting layer, and/or an emitting auxiliary layer.

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials(mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electronic element comprising the compound as an electron transport material.

As another specific example, the same or different compounds of the compound represented by Formula (1) are mixed and used in the organic material layer.

Also, the present invention provides a hole transport layer, an emitting auxiliary layer or an emitting layer composition comprising the compound represented by Formula (1), and provides an organic electronic element comprising a hole transport layer, an emitting auxiliary layer or an emitting layer.

Also, the present invention provides an electronic device comprising a display device including the organic electronic element; and a control unit for driving the display device; In another aspect, the organic electronic element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant (PDA), an electronic dictionary, a point-to-multipoint (PMP), a remote controller, a navigation unit, a game player, various kinds of TVs, and various kinds of computers.

Hereinafter, a synthesis example of the compound represented by Formula (1) of the present invention and a manufacturing example of an organic electronic element of the present invention will be described in detail with reference to Examples, but the present invention is not limited to the following Examples.

### Synthesis Example

The compound represented by Formula (1) according to the present invention (final products) is synthesized by reaction route of Reaction Schemes 1 to 3, but is not limited thereto.

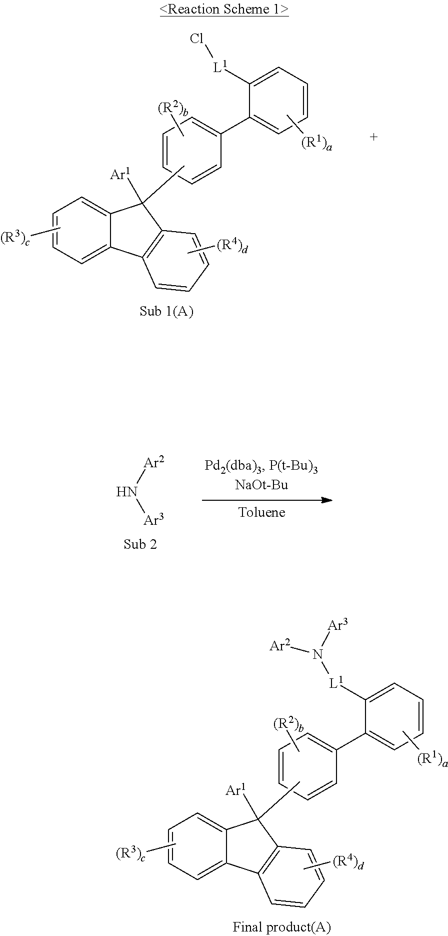

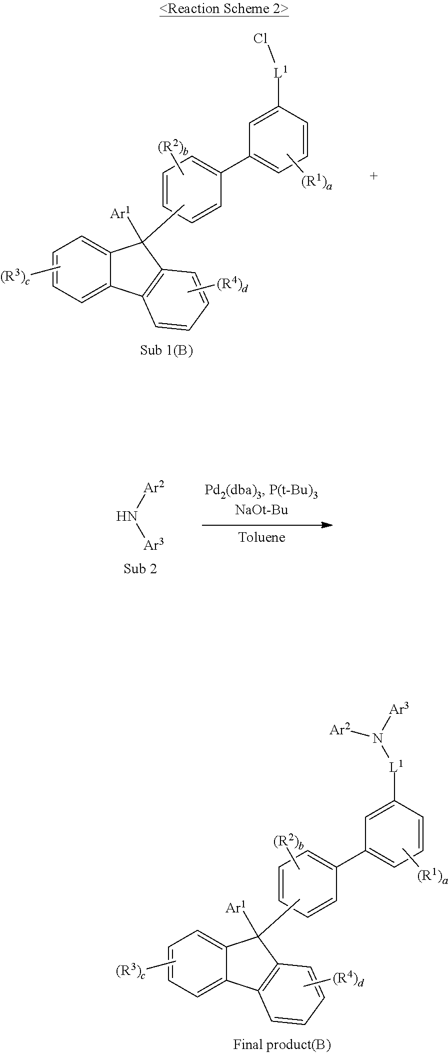

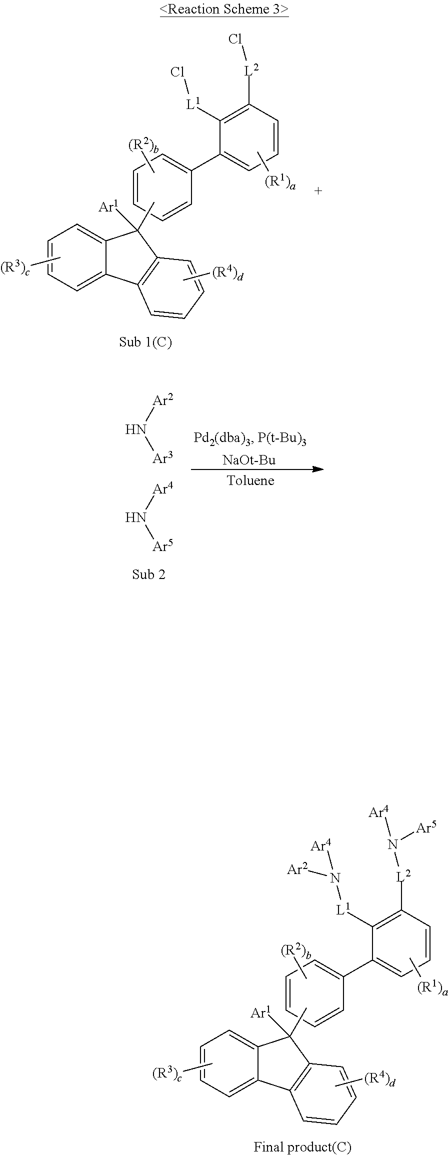

### Synthesis Example of Sub 1

Sub 1 of Reaction Schemes 1 to 3 may be synthesized by the reaction routes of Reaction Schemes 4 to 6, but is not limited thereto.

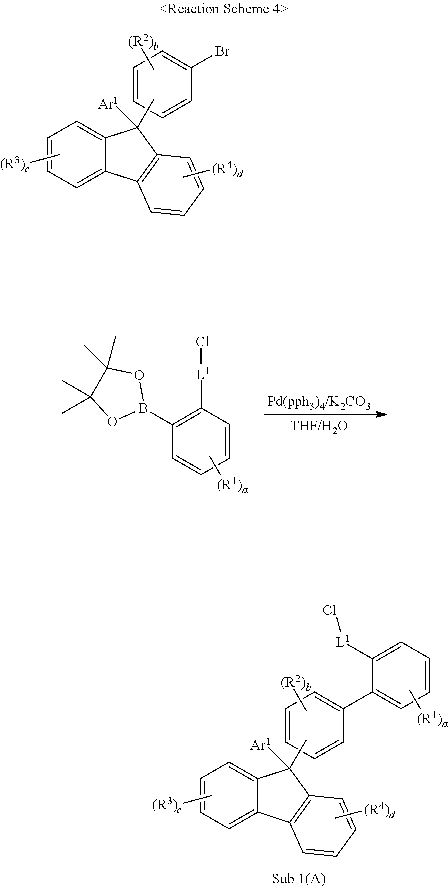

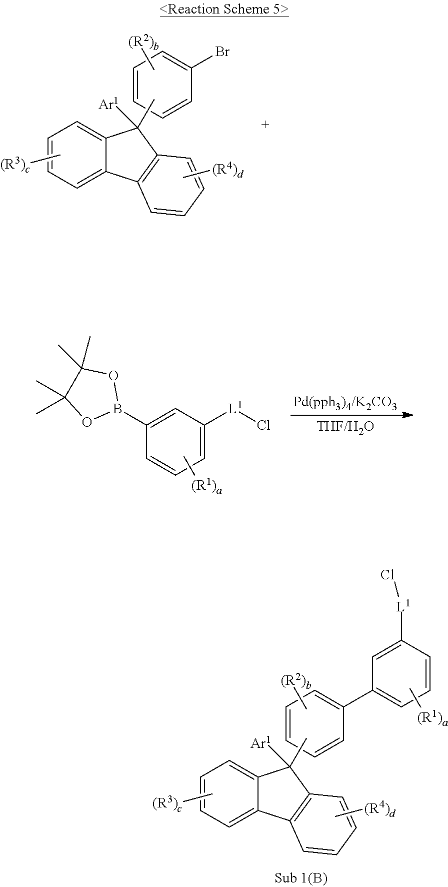

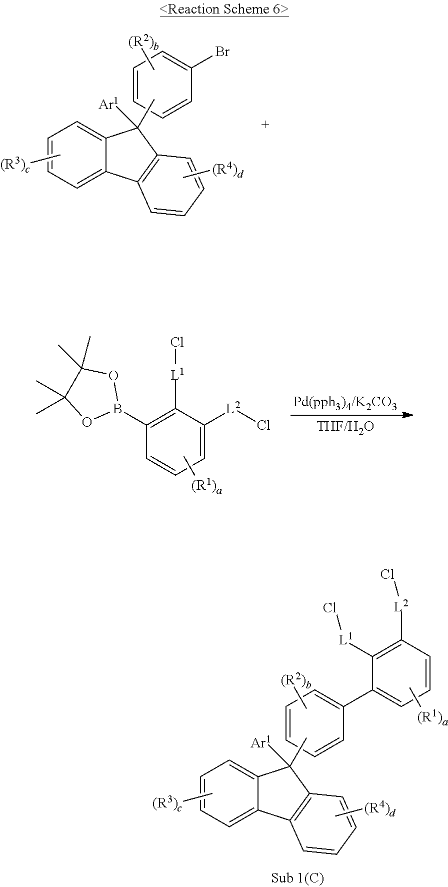

Synthesis examples of specific compounds belonging to Sub 1 are as follows.

### 1. Synthesis Example of Sub 1-1

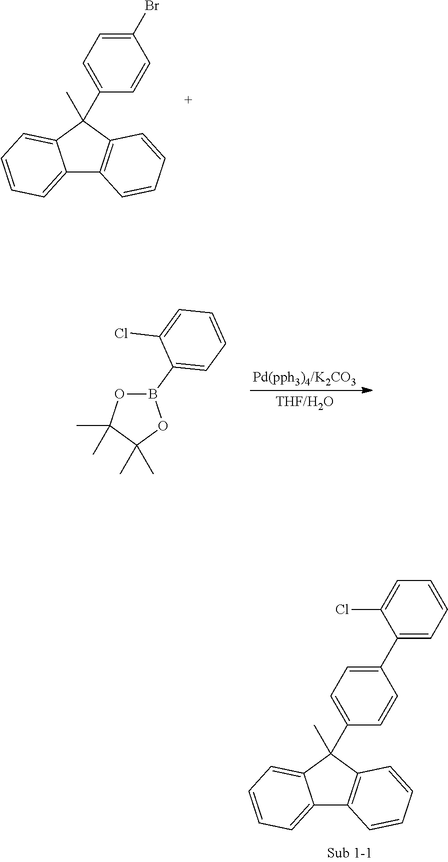 [SMILES: CC1(c2ccc(Br)cc2)c2ccccc2-c2ccccc21 ; "+" ; CC1(C)OB(c2ccccc2Cl)OC1(C)C ; CC1(c2ccc(-c3ccccc3Cl)cc2)c2ccccc2-c2ccccc21]

THF (750 ml) was dissolved in 9-(4-bromophenyl)-9-methyl-9H-fluorene (50 g, 149.1 mmol), 2-(2-chlorophenyl)-4,4,5,5-tetramethyl-1,3,2-dioxaborolane (35.6 g, 149.1 mmol), Pd(PPh₃)₄ (10.34 g, 8.95 mmol), K₂CO₃ (61.8 g, 447.4 mmol), and then water (380 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 44.3 g (yield: 81%) of the product Sub 1-1.

### 2. Synthesis Example of Sub 1-5

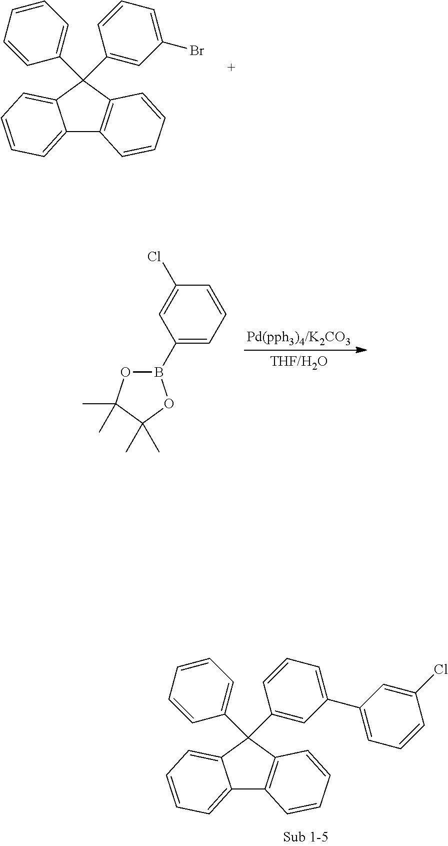

THF (630 ml) was dissolved in 9-(3-bromophenyl)-9-phenyl-9H-fluorene (50 g, 125.8 mmol), 2-(3-chlorophenyl)-4,4,5,5-tetramethyl-1,3,2-dioxaborolane (30 g, 125.8 mmol), Pd(PPh₃)₄ (8.73 g, 7.55 mmol), K₂CO₃ (52.2 g, 377.5 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 43.2 g (yield: 80%) of the product Sub 1-5.

### 3. Synthesis Example of Sub 1-7

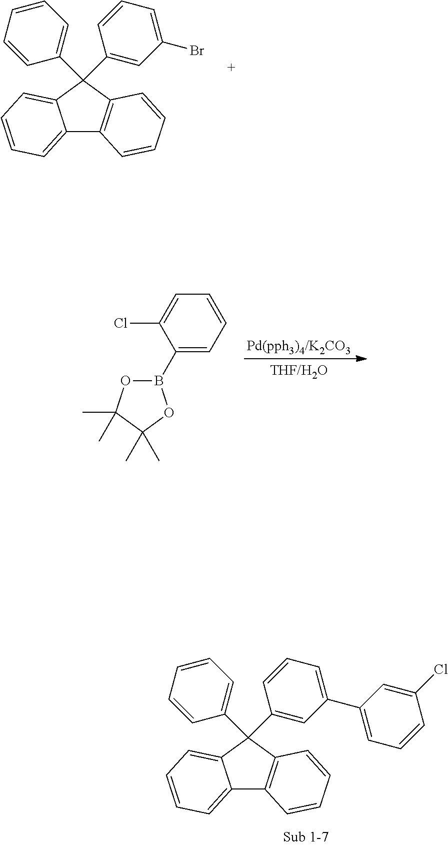

THF (630 ml) was dissolved in 9-(3-bromophenyl)-9-phenyl-9H-fluorene (50 g, 125.8 mmol), 2-(2-chlorophenyl)-4,4,5,5-tetramethyl-1,3,2-dioxaborolane (30 g, 125.8 mmol), Pd(PPh₃)₄ (8.73 g, 7.55 mmol), K₂CO₃ (52.2 g, 377.5 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 42.6 g (yield: 79%) of the product Sub 1-7.

### 4. Synthesis Example of Sub 1-10

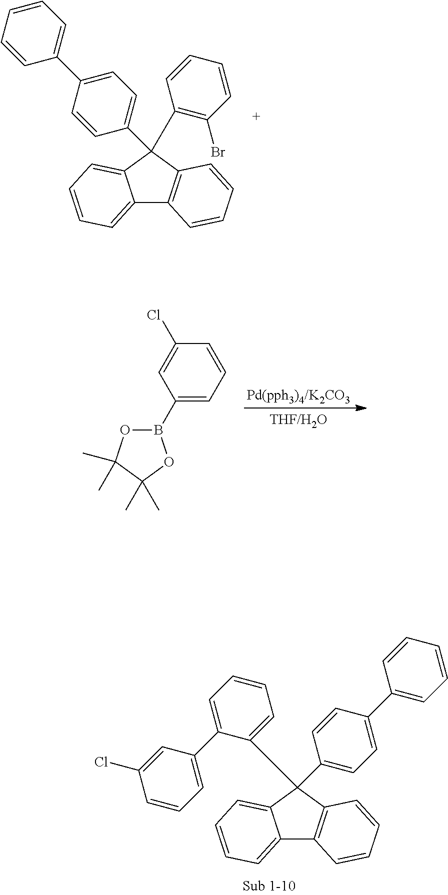

THF (530 ml) was dissolved in 9-([1,1′-biphenyl]-4-yl)-9-(2-bromophenyl)-9H-fluorene (50 g, 105.6 mmol), 2-(3-chlorophenyl)-4,4,5,5-tetramethyl-1,3,2-dioxaborolane (25.2 g, 105.6 mmol), Pd(PPh₃)₄ (7.32 g, 6.34 mmol), K₂C₀₃ (43.8 g, 316.9 mmol) and then water (260 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 36.3 g (yield: 68%) of the product Sub 1-10.

### 5. Synthesis Example of Sub 1-25

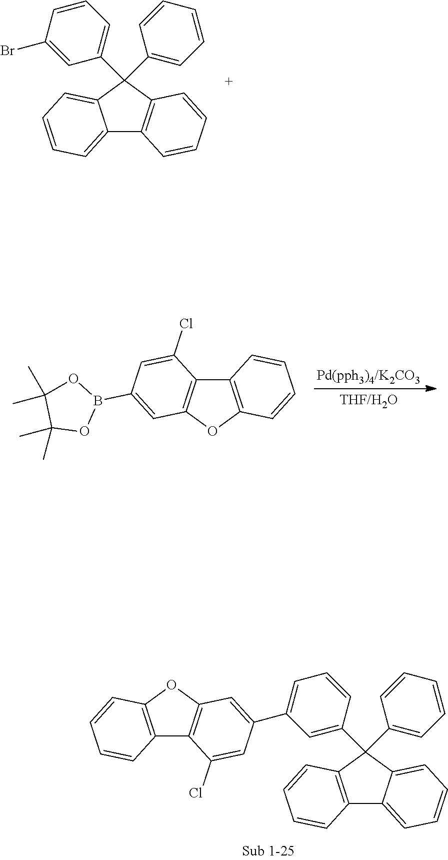

THF (630 ml) was dissolved in 9-(3-bromophenyl)-9-phenyl-9H-fluorene (50 g, 125.8 mmol), 2-(1-chlorodibenzo[b,d]furan-3-yl)-4,4,5,5-tetramethyl-1,3,2-dioxaborolane (41.4 g, 125.8 mmol), Pd(PPh₃)₄ (8.73 g, 7.55 mmol), K₂CO₃ (52.2 g, 377.5 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 52.3 g (yield: 80%) of the product Sub 1-25.

### 6. Synthesis Example of Sub 1-31

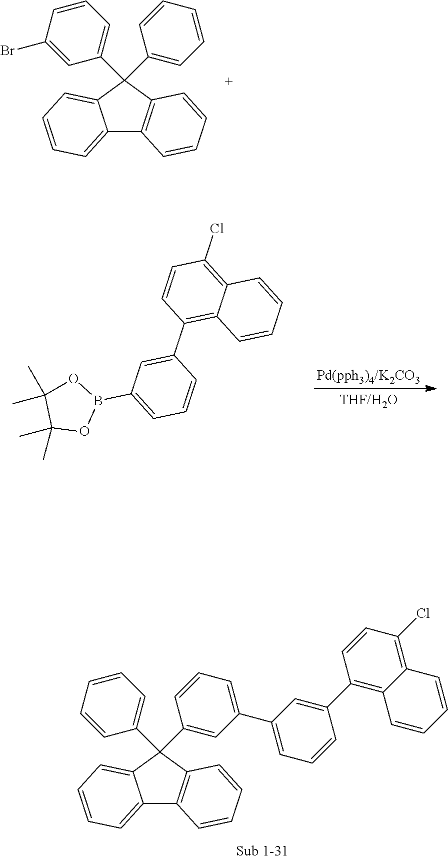

THF (630 ml) was dissolved in 9-(3-bromophenyl)-9-phenyl-9H-fluorene (50 g, 125.8 mmol), 2-(3-(4-chloronaphthalen-1-yl)phenyl)-4,4,5,5-tetramethyl-1,3,2-dioxaborolane (45.9 g, 125.8 mmol), Pd(PPh₃)₄ (8.73 g, 7.55 mmol), K₂CO₃ (52.2 g, 377.5 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 54.5 g (yield: 78%) of the product Sub 1-31.

### 7. Synthesis Example of Sub 1-36

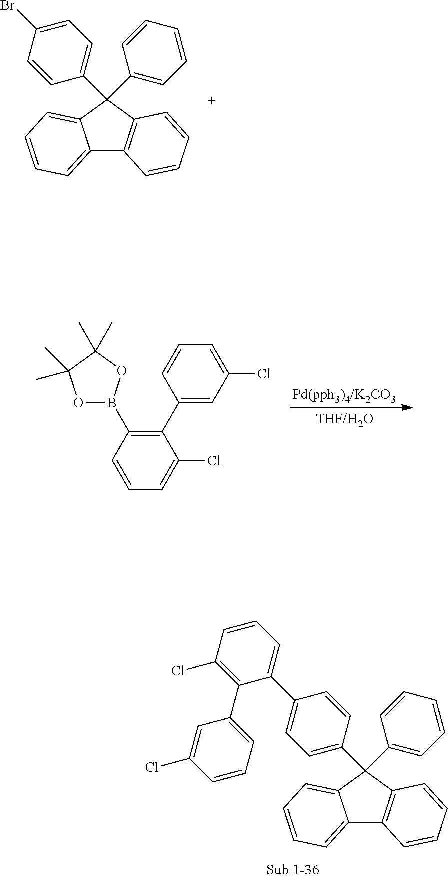

THF (630 ml) was dissolved in 9-(4-bromophenyl)-9-phenyl-9H-fluorene (50 g, 125.8 mmol), 2-(3′,6-dichloro-[1,1′-biphenyl]-2-yl)-4,4,5,5-tetramethyl-1,3,2-dioxaborolane (43.9 g, 125.8 mmol), Pd(PPh₃)₄ (8.73 g, 7.55 mmol), K₂CO₃ (52.2 g, 377.5 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 49.6 g (yield: 73%) of the product Sub 1-36.

### 8. Synthesis Example of Sub 1-52

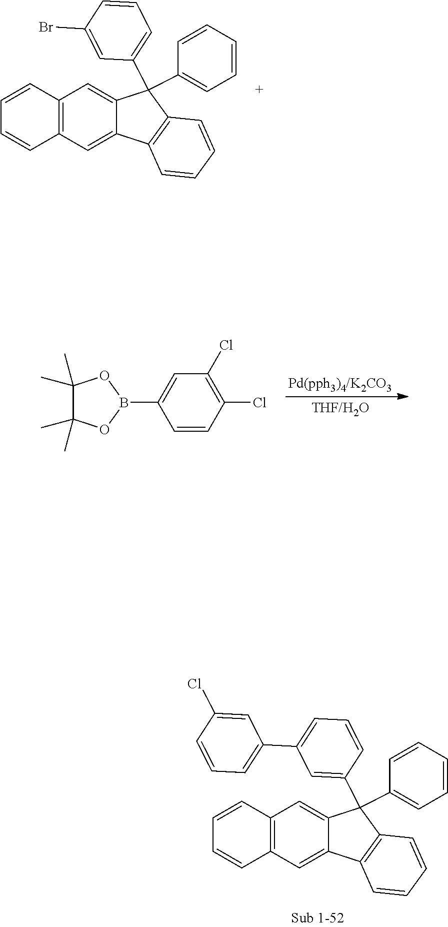

THF (560 ml) was dissolved in 11-(3-bromophenyl)-11-phenyl-11H-benzo[b]fluorene (50 g, 111.8 mmol), 2-(3-chlorophenyl)-4,4,5,5-tetramethyl-1,3,2-dioxaborolane (26.7 g, 111.8 mmol), Pd(PPh₃)₄ (7.75 g, 6.71 mmol), K₂CO₃ (46.3 g, 335.3 mmol) and then water (310 ml) was added, followed by stirring at 90° C. After that, when the reaction was completed, the temperature of the reactant was cooled to room temperature, extracted with MC, and washed with water. The organic layer was dried over MgSO₄ and concentrated, and the resulting organic material was separated by a silica gel filter to obtain 42.8 g (yield: 80%) of the product Sub 1-52.

The compound belonging to Sub 1 may be the following compounds, but is not limited thereto, and Table 1 below shows FD-MS (Field Desorption-Mass Spectrometry) values of the compounds belonging to Sub 1.

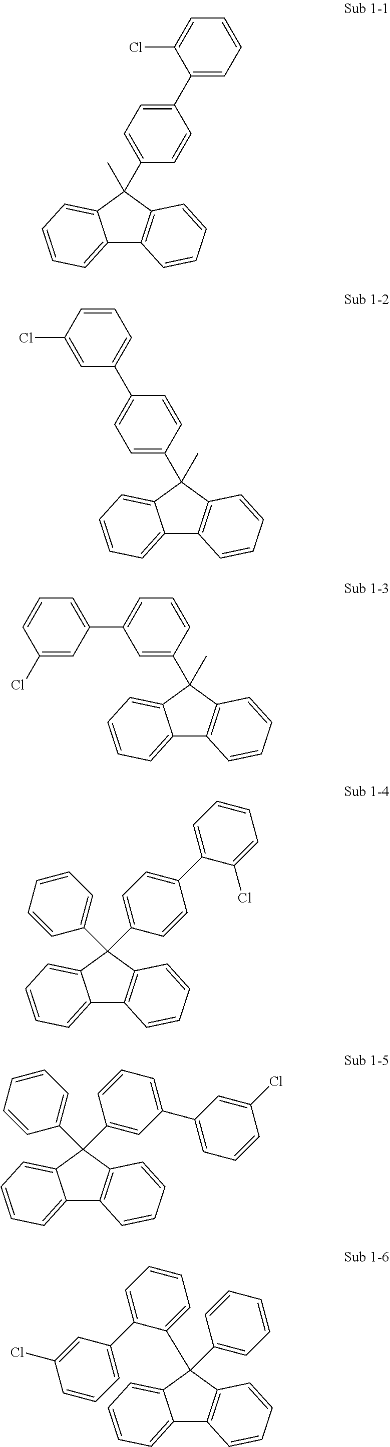

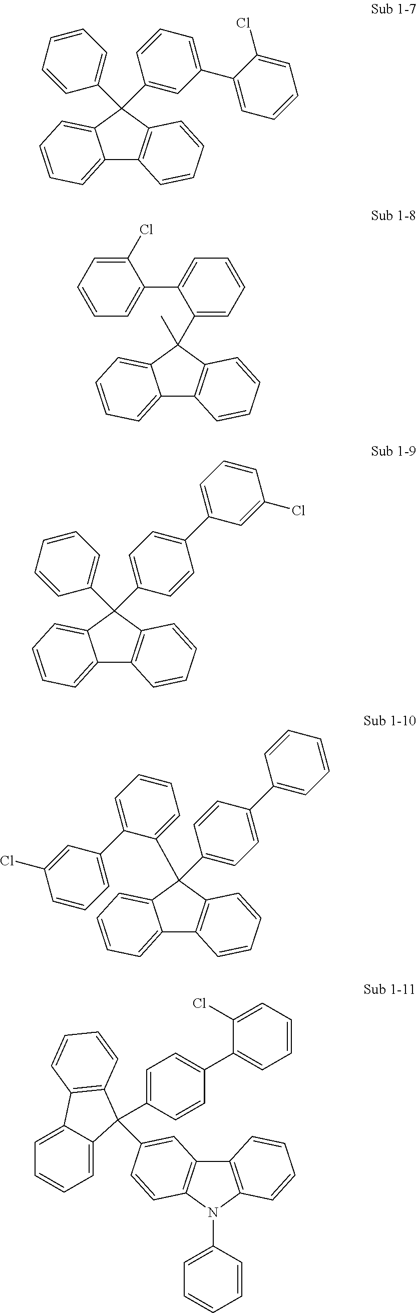

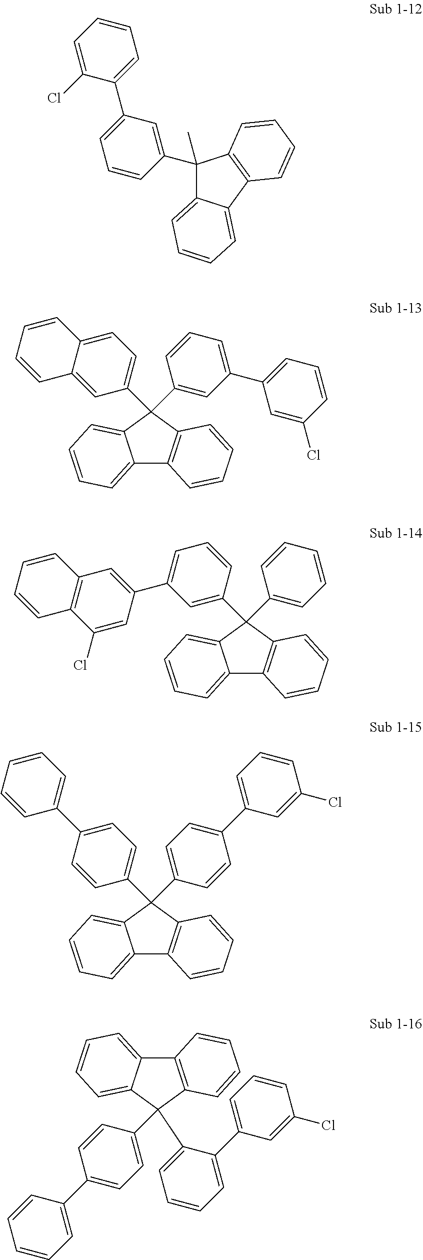 [SMILES: CC1(c2cccc(-c3ccccc3Cl)c2)c2ccccc2-c2ccccc21 ; Clc1cccc(-c2cccc(C3(c4ccc5ccccc5c4)c4ccccc4-c4ccccc43)c2)c1 ; Clc1cc(-c2cccc(C3(c4ccccc4)c4ccccc4-c4ccccc43)c2)cc2ccccc12 ; Clc1cccc(-c2ccc(C3(c4ccc(-c5ccccc5)cc4)c4ccccc4-c4ccccc43)cc2)c1 ; Clc1cccc(-c2ccccc2C2(c3ccc(-c4ccccc4)cc3)c3ccccc3-c3ccccc32)c1]

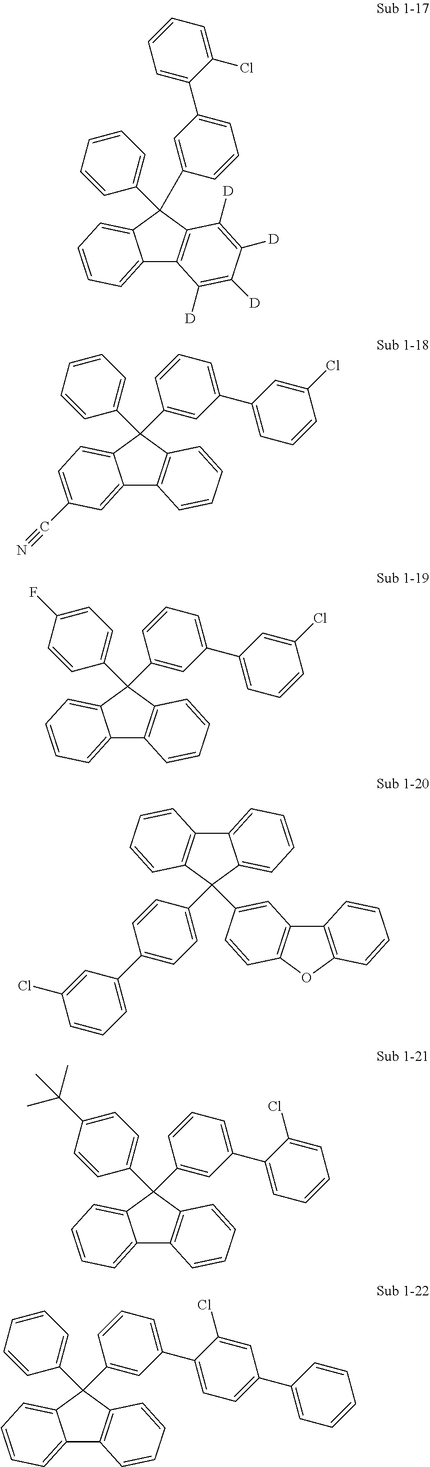

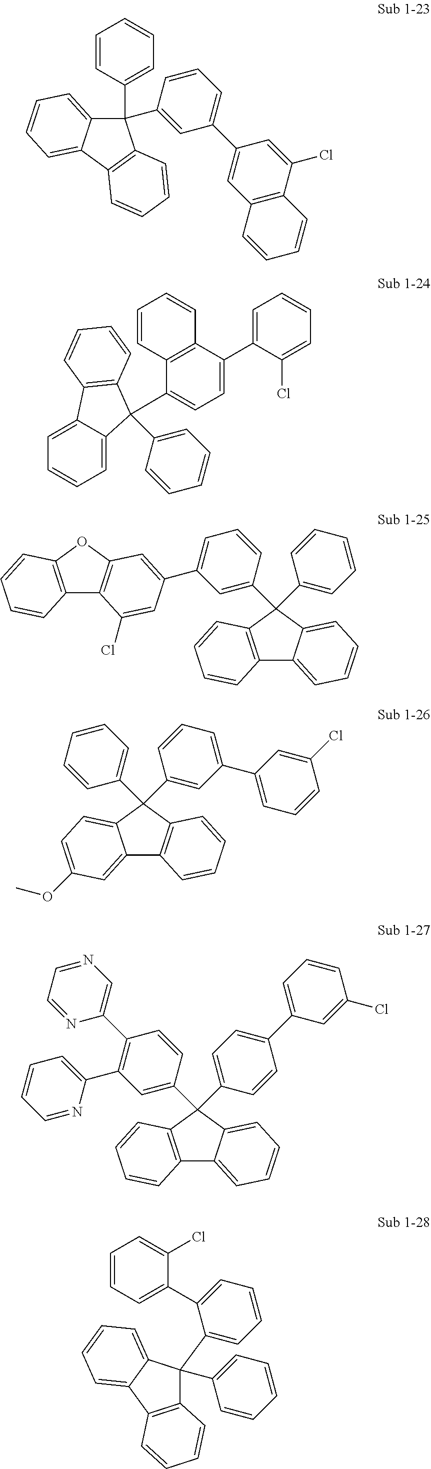

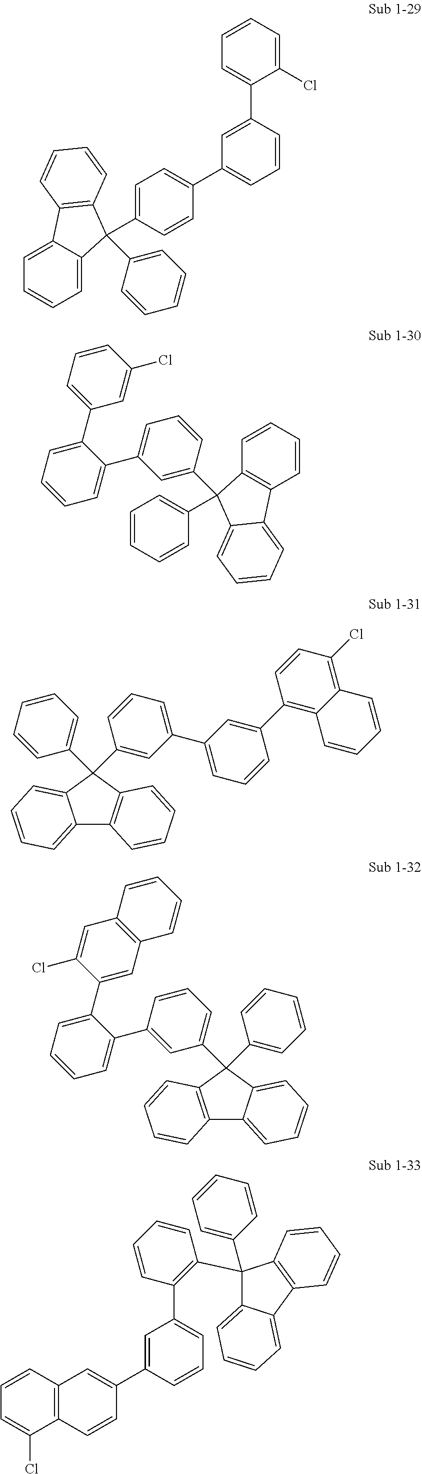

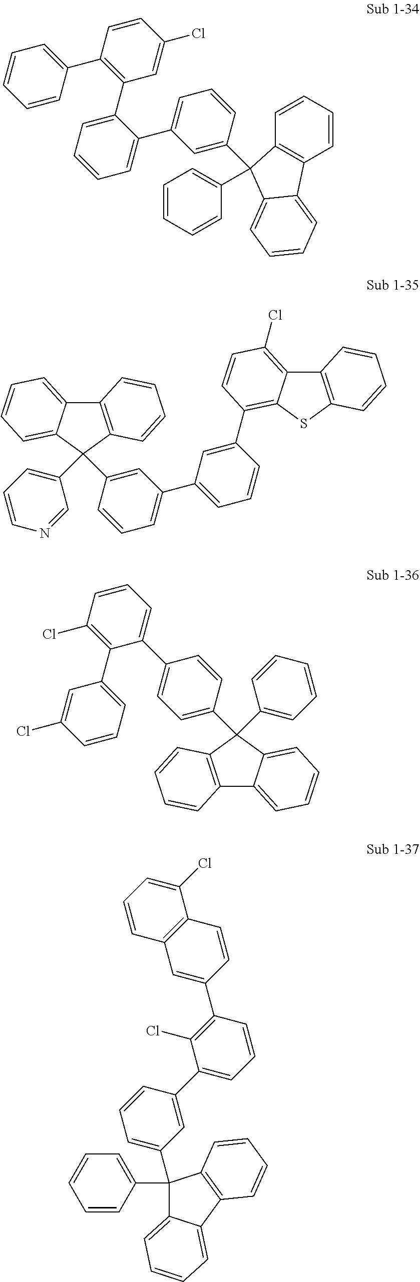 [SMILES: Clc1ccc(-c2ccccc2)c(-c2ccccc2-c2cccc(C3(c4ccccc4)c4ccccc4-c4ccccc43)c2)c1 ; Clc1ccc(-c2cccc(-c3cccc(C4(c5cccnc5)c5ccccc5-c5ccccc54)c3)c2)c2sc3ccccc3c12 ; Clc1cccc(-c2c(Cl)cccc2-c2ccc(C3(c4ccccc4)c4ccccc4-c4ccccc43)cc2)c1 ; Clc1c(-c2cccc(C3(c4ccccc4)c4ccccc4-c4ccccc43)c2)cccc1-c1ccc2c(Cl)cccc2c1]

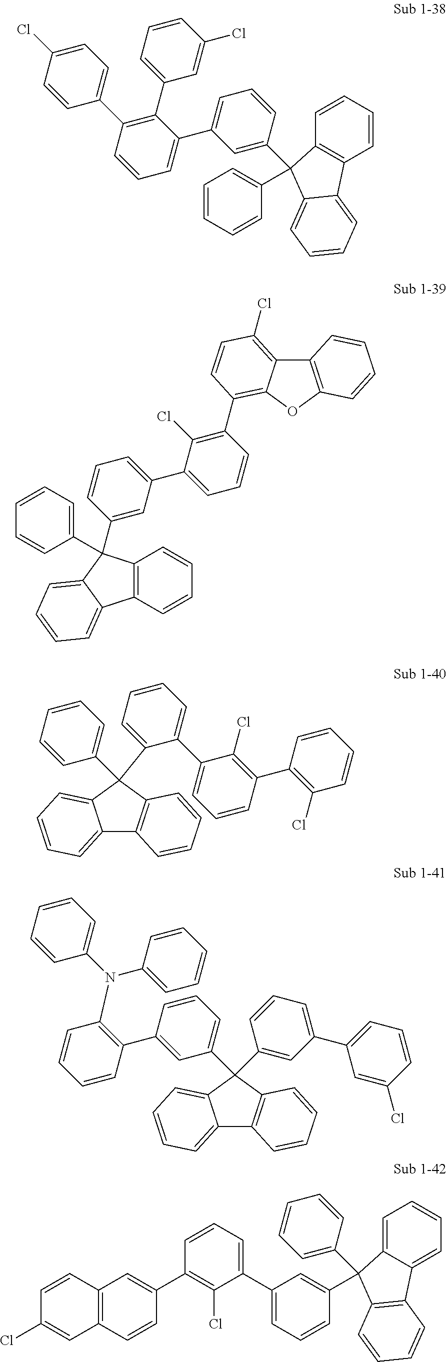

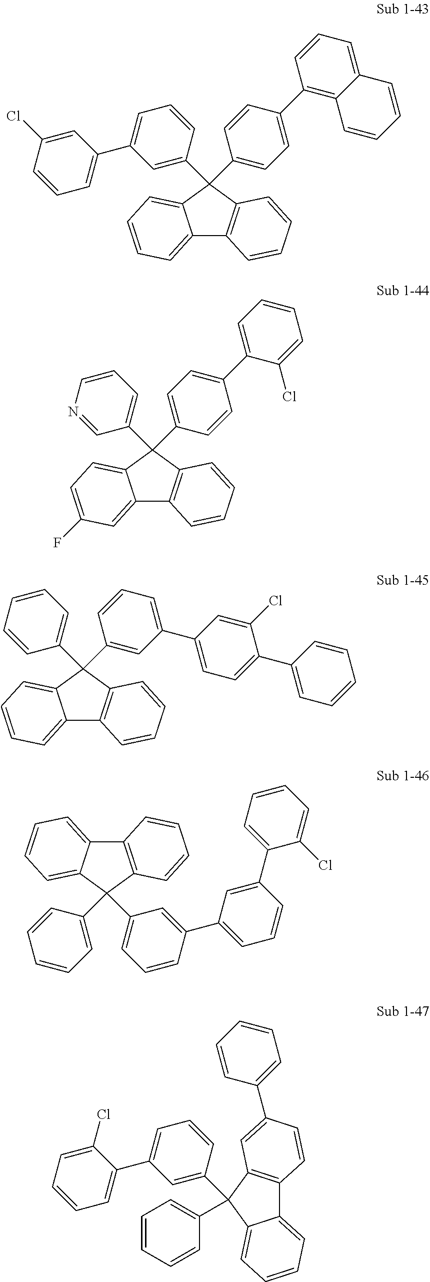

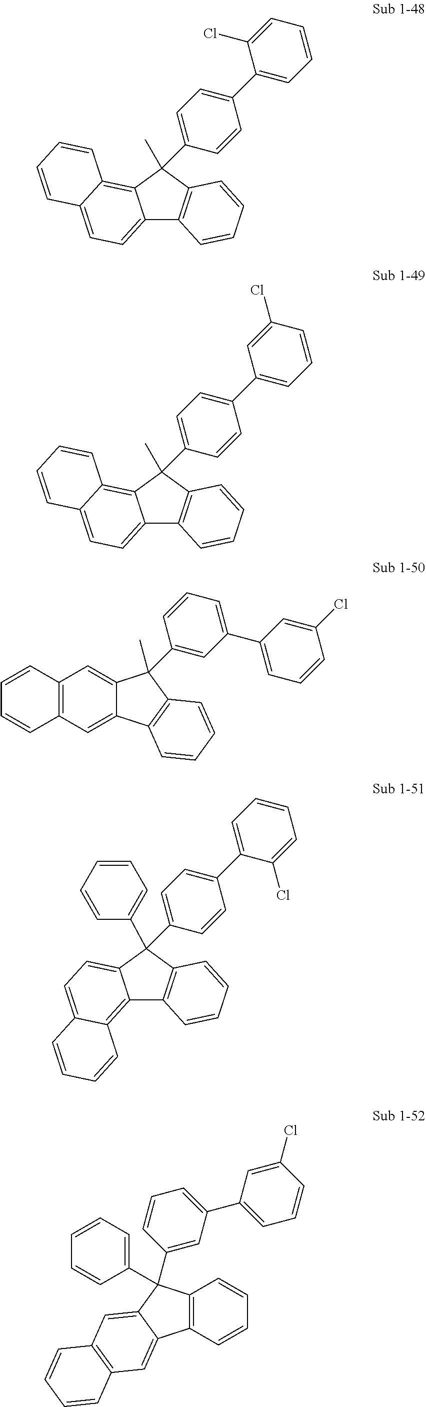 [SMILES: CC1(c2ccc(-c3ccccc3Cl)cc2)c2ccccc2-c2ccc3ccccc3c21 ; CC1(c2ccc(-c3cccc(Cl)c3)cc2)c2ccccc2-c2ccc3ccccc3c21 ; CC1(c2cccc(-c3cccc(Cl)c3)c2)c2ccccc2-c2cc3ccccc3cc21 ; Clc1ccccc1-c1ccc(C2(c3ccccc3)c3ccccc3-c3c2ccc2ccccc32)cc1 ; Clc1cccc(-c2cccc(C3(c4ccccc4)c4ccccc4-c4cc5ccccc5cc43)c2)c1]

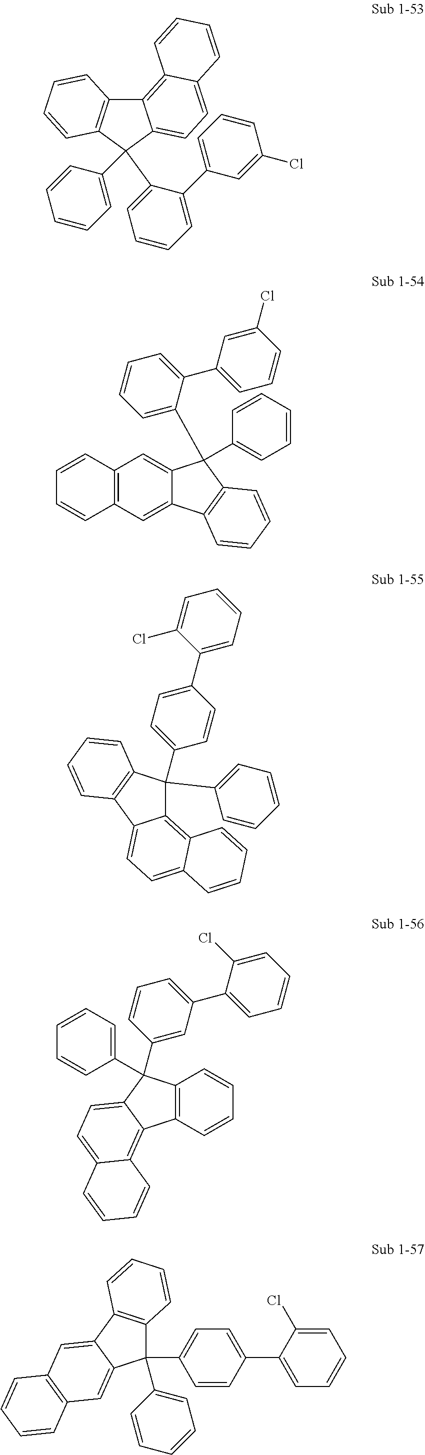

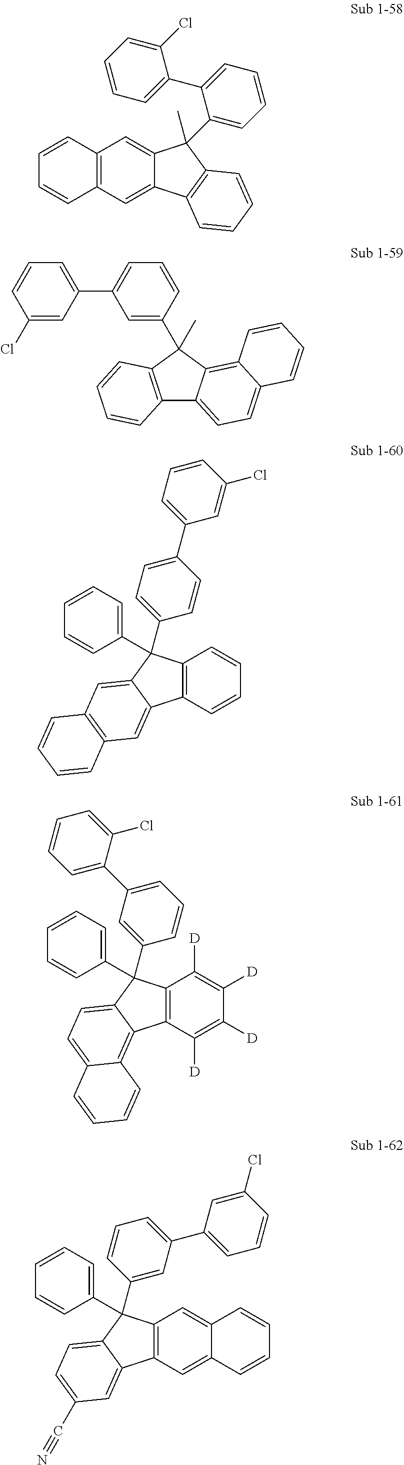

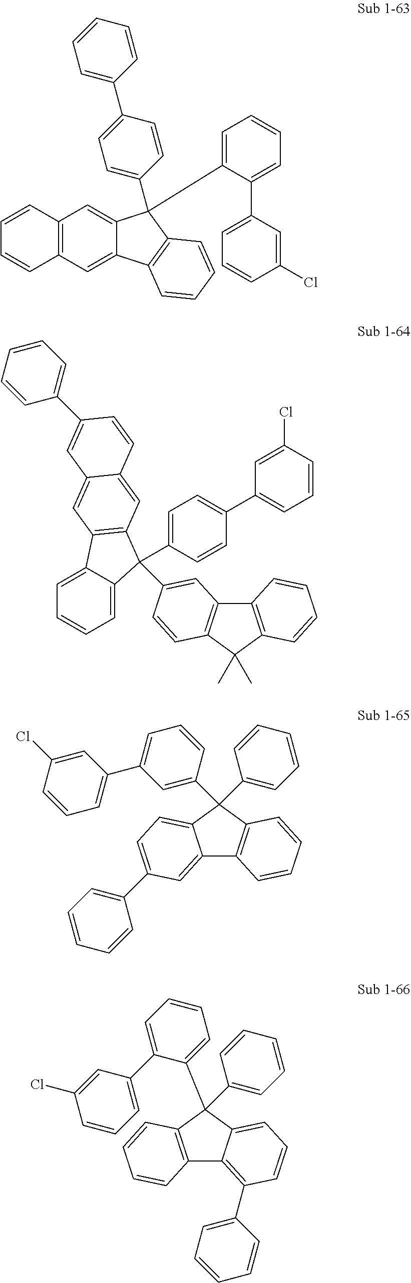

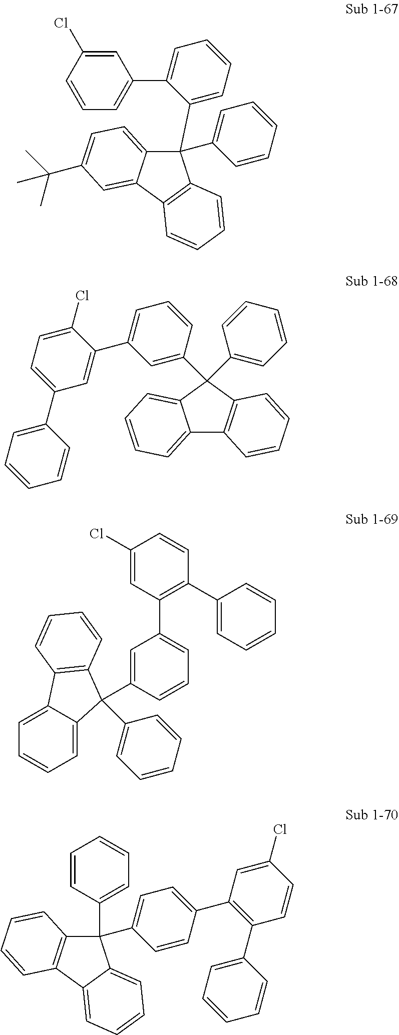 [SMILES: CC(C)(C)c1ccc2c(c1)-c1ccccc1C2(c1ccccc1)c1ccccc1-c1cccc(Cl)c1 ; Clc1ccc(-c2ccccc2)cc1-c1cccc(C2(c3ccccc3)c3ccccc3-c3ccccc32)c1 ; Clc1ccc(-c2ccccc2)c(-c2cccc(C3(c4ccccc4)c4ccccc4-c4ccccc43)c2)c1 ; Clc1ccc(-c2ccccc2)c(-c2ccc(C3(c4ccccc4)c4ccccc4-c4ccccc43)cc2)c1]

TABLE 1

| com- | | com- | |
|---|---|---|---|
| pound | FD-MS | pound | FD-MS |
| Sub 1-1 | m/z = 366.12(C₂₆H₁₉Cl = 366.89) | Sub 1-2 | m/z = 366.12(C₂₆H₁₉Cl = 366.89) |
| Sub 1-3 | m/z = 366.12(C₂₆H₁₉Cl = 366.89) | Sub 1-4 | m/z = 428.13(C₃₁H₂₁Cl = 428.96) |
| Sub 1-5 | m/z = 428.13(C₃₁H₂₁Cl = 428.96) | Sub 1-6 | m/z = 428.13(C₃₁H₂₁Cl = 428.96) |
| Sub 1-7 | m/z = 428.13(C₃₁H₂₁Cl = 428.96) | Sub 1-8 | m/z = 366.12(C₂₆H₁₉Cl = 366.89) |
| Sub 1-9 | m/z = 428.13(C₃₁H₂₁Cl = 428.96) | Sub 1-10 | m/z = 504.16(C₃₇H₂₅Cl = 505.06) |
| Sub 1-11 | m/z = 593.19(C₄₃H₂₈ClN = 594.15) | Sub 1-12 | m/z = 366.12(C₂₆H₁₉Cl = 366.89) |
| Sub 1-13 | m/z = 478.15(C₃₅H₂₃Cl = 479.02) | Sub 1-14 | m/z = 478.15(C₃₅H₂₃Cl = 479.02) |
| Sub 1-15 | m/z = 504.16(C₃₇H₂₅Cl = 505.06) | Sub 1-16 | m/z = 504.16(C₃₇H₂₅Cl = 505.06) |
| Sub 1-17 | m/z = 432.16(C₃₁H₁₇D₄Cl = 432.98) | Sub 1-18 | m/z = 453.13(C₃₂H₂₀ClN = 453.97) |
| Sub 1-19 | m/z = 446.12(C₃₁H₂₀ClF = 446.95) | Sub 1-20 | m/z = 518.14(C₃₇H₂₃ClO = 519.04) |
| Sub 1-21 | m/z = 484.2(C₃₅H₂₉Cl = 485.07) | Sub 1-22 | m/z = 504.16(C₃₇H₂₅Cl = 505.06) |
| Sub 1-23 | m/z = 478.15(C₃₅H₂₃Cl = 479.02) | Sub 1-24 | m/z = 478.15(C₃₅H₂₃Cl = 479.02) |
| Sub 1-25 | m/z = 518.14(C₃₇H₂₃ClO = 519.04) | Sub 1-26 | m/z = 458.14(C₃₂H₂₃ClO = 458.99) |
| Sub 1-27 | m/z = 583.18(C₄₀H₂₆ClN₃ = 584.12) | Sub 1-28 | m/z = 428.13(C₃₁H₂₁Cl = 428.96) |
| Sub 1-29 | m/z = 504.16(C₃₇H₂₅Cl = 505.06) | Sub 1-30 | m/z = 504.16(C₃₇H₂₅Cl = 505.06) |
| Sub 1-31 | m/z = 554.18(C₄₁H₂₇Cl = 555.12) | Sub 1-32 | m/z = 554.18(C₄₁H₂₇Cl = 555.12) |
| Sub 1-33 | m/z = 554.18(C₄₁H₂₇Cl = 555.12) | Sub 1-34 | m/z = 580.2(C₄₃H₂₉Cl = 581.16) |
| Sub 1-35 | m/z = 611.15(C₄₂H₂₆ClNS = 612.19) | Sub 1-36 | m/z = 538.13(C₃₇H₂₄Cl₂ = 539.5) |
| Sub 1-37 | m/z = 588.14(C₄₁H₂₆Cl₂ = 589.56) | Sub 1-38 | m/z = 614.16(C₄₃H₂₈Cl₂ = 615.6) |
| Sub 1-39 | m/z = 628.14(C₄₃H₂₆Cl₂O = 629.58) | Sub 1-40 | m/z = 538.13(C₃₇H₂₄Cl₂ = 539.5) |
| Sub 1-41 | m/z = 671.24(C₄₉H₃₄ClN = 672.27) | Sub 1-42 | m/z = 588.14(C₄₁H₂₆Cl₂ = 589.56) |
| Sub 1-43 | m/z = 554.18(C₄₁H₂₇Cl = 555.12) | Sub 1-44 | m/z = 447.12(C₃₀H₁₉ClFN = 447.94) |
| Sub 1-45 | m/z = 504.16(C₃₇H₂₅Cl = 505.06) | Sub 1-46 | m/z = 504.16(C₃₇H₂₅Cl = 505.06) |
| Sub 1-47 | m/z = 504.16(C₃₇H₂₅Cl = 505.06) | Sub 1-48 | m/z = 416.13(C₃₀H₂₁Cl = 416.95) |
| Sub 1-49 | m/z = 416.13(C₃₀H₂₁Cl = 416.95) | Sub 1-50 | m/z = 416.13(C₃₀H₂₁Cl = 416.95) |
| Sub 1-51 | m/z = 478.15(C₃₅H₂₃Cl = 479.02) | Sub 1-52 | m/z = 478.15(C₃₅H₂₃Cl = 479.02) |
| Sub 1-53 | m/z = 478.15(C₃₅H₂₃Cl = 479.02) | Sub 1-54 | m/z = 478.15(C₃₅H₂₃Cl = 479.02) |
| Sub 1-55 | m/z = 478.15(C₃₅H₂₃Cl = 479.02) | Sub 1-56 | m/z = 478.15(C₃₅H₂₃Cl = 479.02) |
| Sub 1-57 | m/z = 478.15(C₃₅H₂₃Cl = 479.02) | Sub 1-58 | m/z = 416.13(C₃₀H₂₁Cl = 416.95) |
| Sub 1-59 | m/z = 416.13(C₃₀H₂₁Cl = 416.95) | Sub 1-60 | m/z = 478.15(C₃₅H₂₃Cl = 479.02) |
| Sub 1-61 | m/z = 482.17(C₃₅H₁₉D₄Cl = 483.04) | Sub 1-62 | m/z = 503.14(C₃₆H₂₂ClN = 504.03) |
| Sub 1-63 | m/z = 554.18(C₄₁H₂₇Cl = 555.12) | Sub 1-64 | m/z = 670.24(C₅₀H₃₅Cl = 671.28) |
| Sub 1-65 | m/z = 504.16(C₃₇H₂₅Cl = 505.06) | Sub 1-66 | m/z = 504.16(C₃₇H₂₅Cl = 505.06) |
| Sub 1-67 | m/z = 484.2(C₃₅H₂₉Cl = 485.07) | Sub 1-68 | m/z = 504.16(C₃₇H₂₅Cl = 505.06) |
| Sub 1-69 | m/z = 504.16(C₃₇H₂₅Cl = 505.06) | Sub 1-70 | m/z = 504.16(C₃₇H₂₅Cl = 505.06) |

### Synthesis Example of Sub 2

Sub 2 of Reaction Schemes 1 to 3 may be synthesized by the reaction route of Reaction Scheme 7, but is not limited thereto.

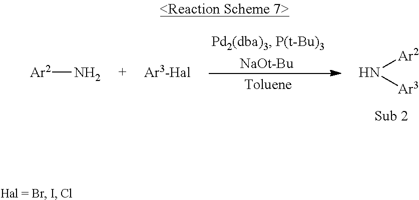

Synthesis examples of specific compounds belonging to Sub 2 are as follows.

### 1. Synthesis Example of Sub 2-1

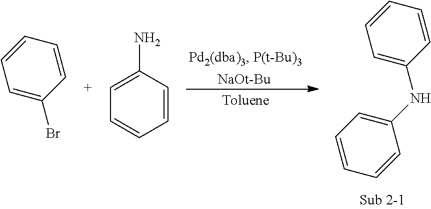

aniline (29.65 g, 318.45 mmol), Pd₂(dba)₃ (8.74 g, 9.55 mmol), P(t-Bu)₃ (6.44 g, 31.84 mmol), NaOt-Bu (61.20 g, 636.9 mmol), Toluene (900 mL) were added to bromobenzene (50 g, 318.45 mmol) and reacted for 2 hours. When the reaction is complete, the mixture is extracted with water, the organic layer is dried over MgSO₄ and concentrated, and the resulting compound was recrystallized by silicagel column to obtain Sub 2-1 (40.41 g, yield: 75%).

### 2. Synthesis Example of Sub 2-4

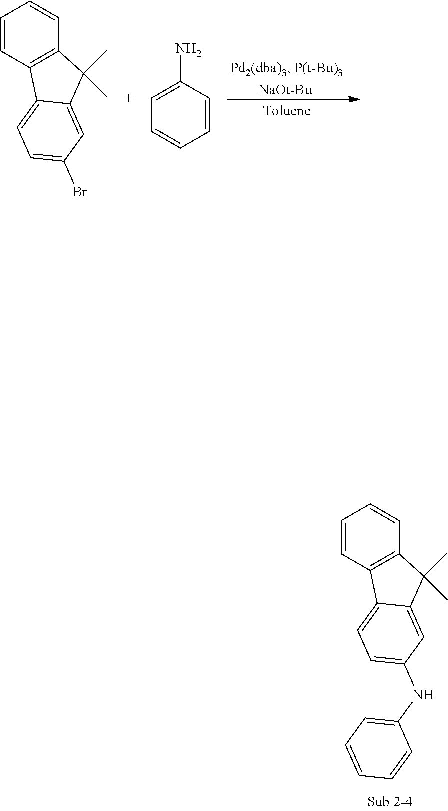

aniline (17 g, 183 mmol), Pd₂(dba)₃ (5.03 g, 5.49 mmol), P(t-Bu)₃ (2.22 g, 10.98 mmol), NaOt-Bu (35.2 g, 366.1 mmol), Toluene (900 mL) were added to 2-bromo-9,9-dimethyl-9H-fluorene (50 g, 183 mmol) and reacted for 2 hours. When the reaction is complete, the mixture is extracted with water, the organic layer is dried over MgSO₄ and concentrated, and the resulting compound was recrystallized by silicagel column to obtain Sub 2-4 (38.1 g, yield: 73%).

### 3. Synthesis Example of Sub 2-11

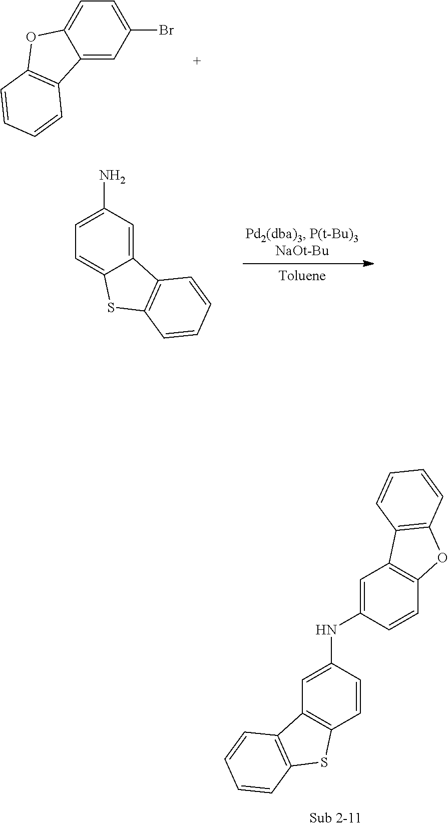 [SMILES: Brc1ccc2oc3ccccc3c2c1 ; Nc1ccc2sc3ccccc3c2c1 ; c1ccc2c(c1)oc1ccc(Nc3ccc4sc5ccccc5c4c3)cc12]

dibenzo[b,d]thiophen-2-amine (40.3 g, 202.4 mmol), Pd₂(dba)₃ (5.56 g, 6.07 mmol), P(t-Bu)₃ (2.46 g, 12.14 mmol), NaOt-Bu (38.9 g, 404.7 mmol), Toluene (1000 mL) were added to 2-bromodibenzo[b,d]furan (50 g, 202.4 mmol) and reacted for 2 hours. When the reaction is complete, the mixture is extracted with water, the organic layer is dried over MgSO₄ and concentrated, and the resulting compound was recrystallized by silicagel column to obtain Sub 2-11 (51.8 g, yield: 70%).

### 4. Synthesis Example of Sub 2-14

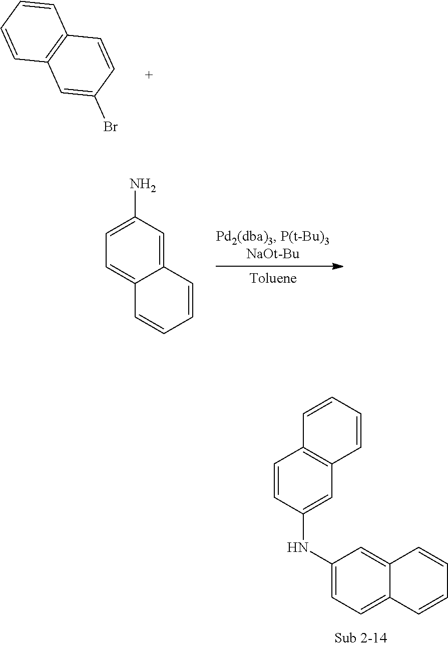 [SMILES: Brc1ccc2ccccc2c1 ; Nc1ccc2ccccc2c1 ; c1ccc2cc(Nc3ccc4ccccc4c3)ccc2c1]

naphthalen-2-amine (34.6 g, 241.5 mmol), Pd₂(dba)₃ (6.63 g, 7.24 mmol), P(t-Bu)₃ (2.93 g, 14.49 mmol), NaOt-Bu (46.4 g, 482.9 mmol), Toluene (1200 mL) were added to 2-bromonaphthalene (50 g, 241.5 mmol) and reacted for 2 hours. When the reaction is complete, the mixture is extracted with water, the organic layer is dried over MgSO₄ and concentrated, and the resulting compound was recrystallized by silicagel column to obtain Sub 2-14 (48.1 g, yield: 74%).

### 5. Synthesis Example of Sub 2-22

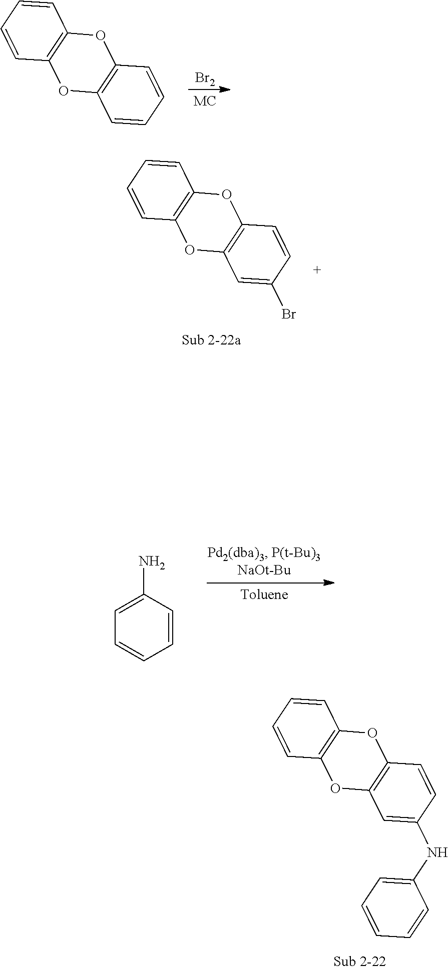

### (1) Synthesis of Sub 2-22a

After dissolving dibenzo[b,e][1,4]dioxine (50 g, 271.45 mmol) in 500 ml of dichloromethane, the mixture was stirred at 0° C. for 30 minutes. Br₂ (43.16 g, 271.45 mmol) was slowly added dropwise, and the reaction solution was stirred at room temperature for 6 hours. When the reaction is complete, the mixture is extracted with water, the organic layer is dried over MgSO₄ and concentrated, the resulting compound was subjected to silicagel column and recrystallization to obtain Sub 2-22a (64.27 g, yield: 90%) as a product.

### (2) Synthesis of Sub 2-22

aniline (17.69 g, 190.04 mmol), Pd₂(dba)₃ (5.22 g, 5.70 mmol), P(t-Bu)₃ (3.84 g, 19.00 mmol), NaOt-Bu (36.52 g, 380.08 mmol), Toluene (600 mL) were added to Sub 2-22a (50 g, 190.04 mmol) and reacted for 2 hours. When the reaction is complete, the mixture is extracted with water, the organic layer is dried over MgSO₄ and concentrated, and the resulting compound was recrystallized by silicagel column to obtain Sub 2-22 (41.33 g, yield: 79%).

### 6. Synthesis Example of Sub 2-36

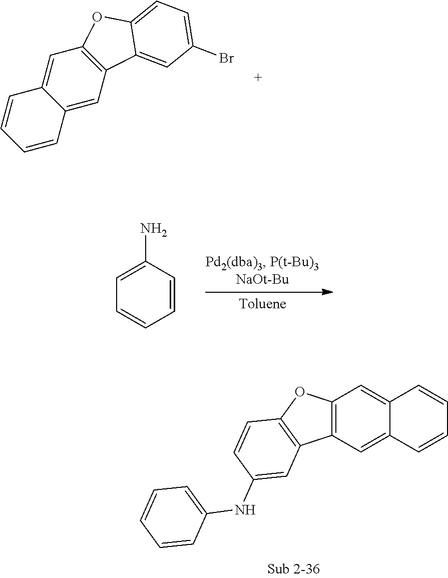 [SMILES: Brc1ccc2oc3cc4ccccc4cc3c2c1 ; Nc1ccccc1 ; c1ccc(Nc2ccc3oc4cc5ccccc5cc4c3c2)cc1]

aniline (15.7 g, 168.3 mmol), Pd₂(dba)₃ (4.62 g, 5.05 mmol), P(t-Bu)₃ (2.04 g, 10.1 mmol), NaOt-Bu (32.3 g, 336.5 mmol), Toluene (850 mL) were added to 2-bromonaphtho[2,3-b]benzofuran (50 g, 168.3 mmol) and reacted for 2 hours. When the reaction is complete, the mixture is extracted with water, the organic layer is dried over MgSO₄ and concentrated, and the resulting compound was recrystallized by silicagel column to obtain Sub 2-36 (38 g, yield: 73%).

### 7. Synthesis Example of Sub 2-39

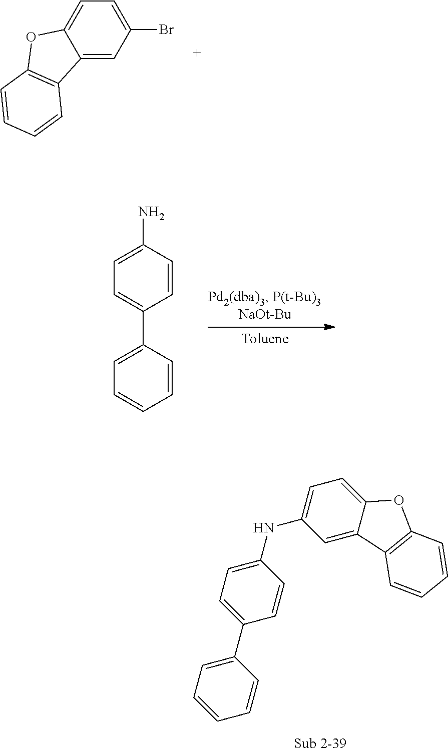 [SMILES: Brc1ccc2oc3ccccc3c2c1 ; Nc1ccc(-c2ccccc2)cc1 ; c1ccc(-c2ccc(Nc3ccc4oc5ccccc5c4c3)cc2)cc1]

[1,1′-biphenyl]-4-amine (34.2 g, 202.4 mmol), Pd₂(dba)₃ (5.56 g, 6.07 mmol), P(t-Bu)₃ (2.46 g, 12.14 mmol), NaOt-Bu (38.9 g, 404.7 mmol), Toluene (1000 mL) were added to 2-bromodibenzo[b,d]furan (50 g, 202.4 mmol) and reacted for 2 hours. When the reaction is complete, the mixture is extracted with water, the organic layer is dried over MgSO₄ and concentrated, and the resulting compound was recrystallized by silicagel column to obtain Sub 2-39 (48.9 g, yield: 72%).

### 8. Synthesis Example of Sub 2-57

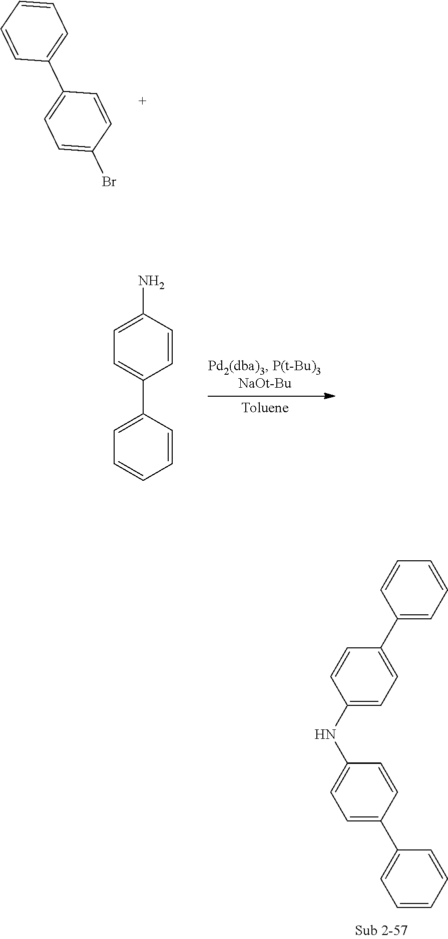 [SMILES: Brc1ccc(-c2ccccc2)cc1 ; Nc1ccc(-c2ccccc2)cc1 ; c1ccc(-c2ccc(Nc3ccc(-c4ccccc4)cc3)cc2)cc1]

[1,1′-biphenyl]-4-amine (36.3 g, 214.5 mmol), Pd₂(dba)₃ (5.89 g, 6.43 mmol), P(t-Bu)₃ (2.6 g, 12.87 mmol), NaOt-Bu (41.2 g, 429 mmol), Toluene (1000 mL) were added to 4-bromo-1,1′-biphenyl (50 g, 214.5 mmol) and reacted for 2 hours. When the reaction is complete, the mixture is extracted with water, the organic layer is dried over MgSO₄ and concentrated, and the resulting compound was recrystallized by silicagel column to obtain Sub 2-57 (48.3 g, yield: 70%).

The compound belonging to Sub 2 may be the following compounds, but is not limited thereto, and Table 1 below shows FD-MS (Field Desorption-Mass Spectrometry) values of the compounds belonging to Sub 2.

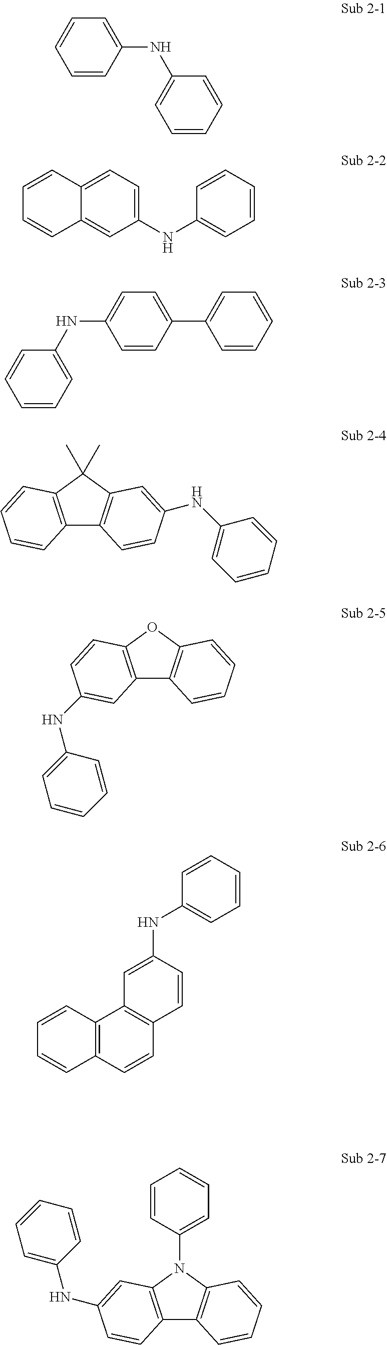

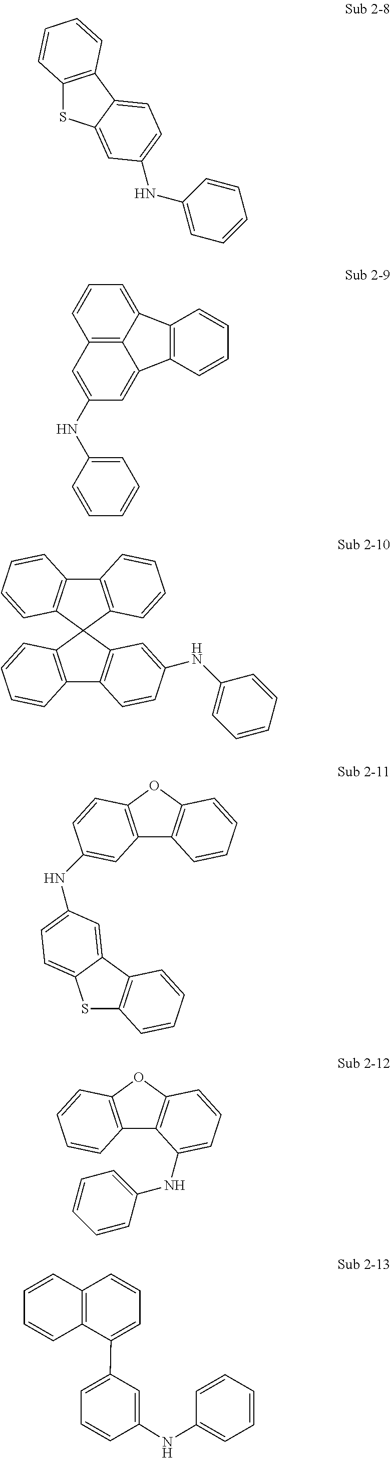

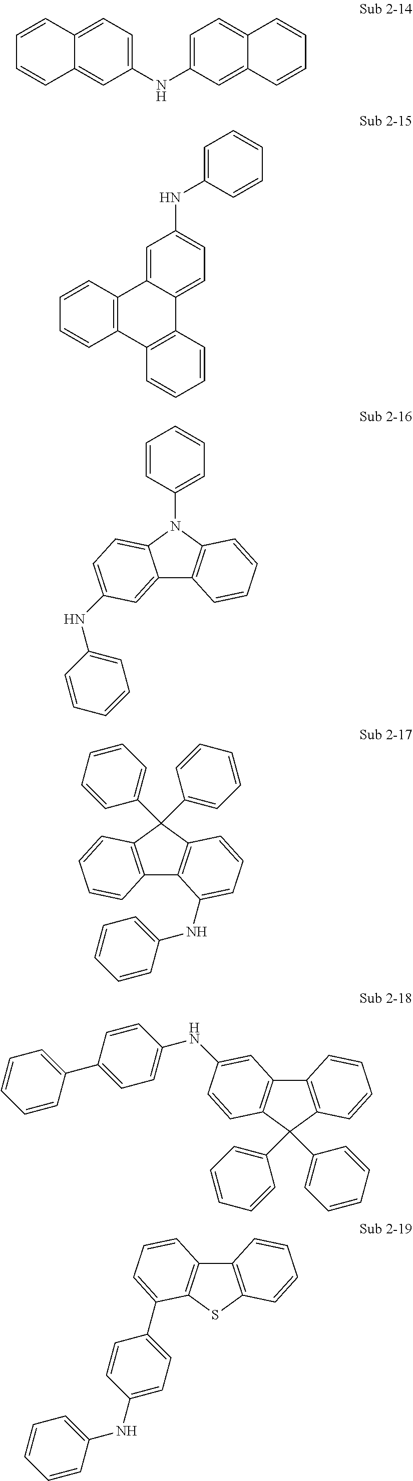

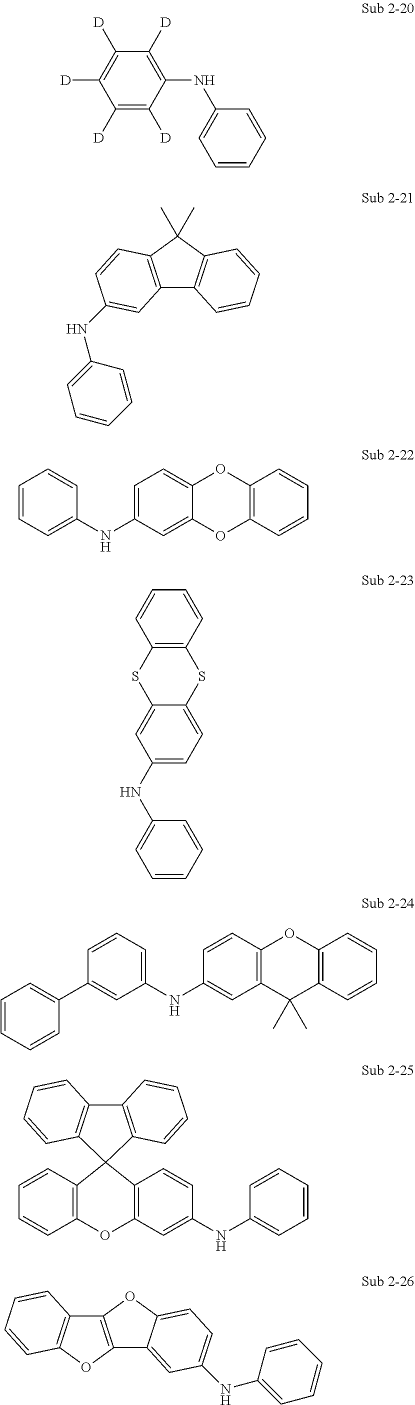

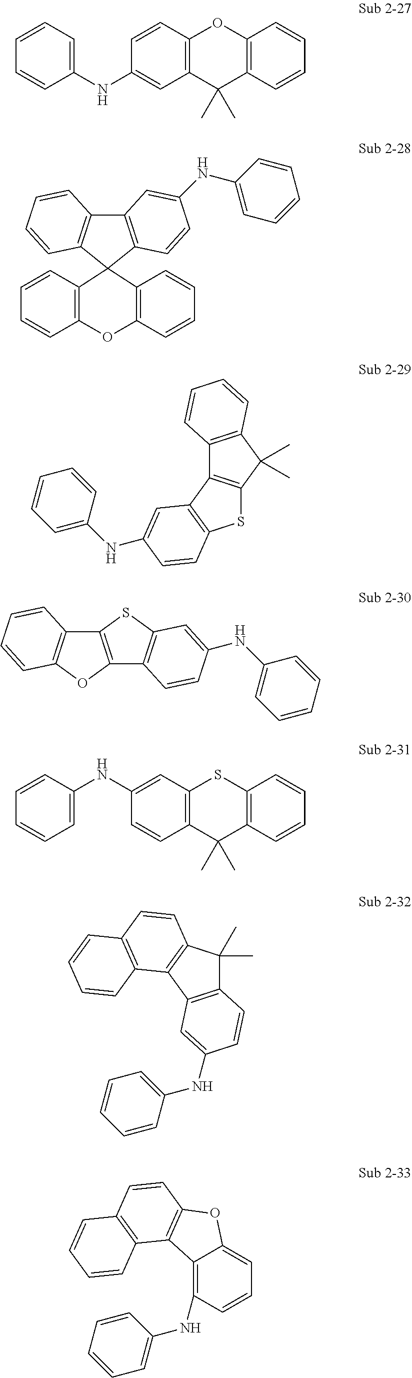

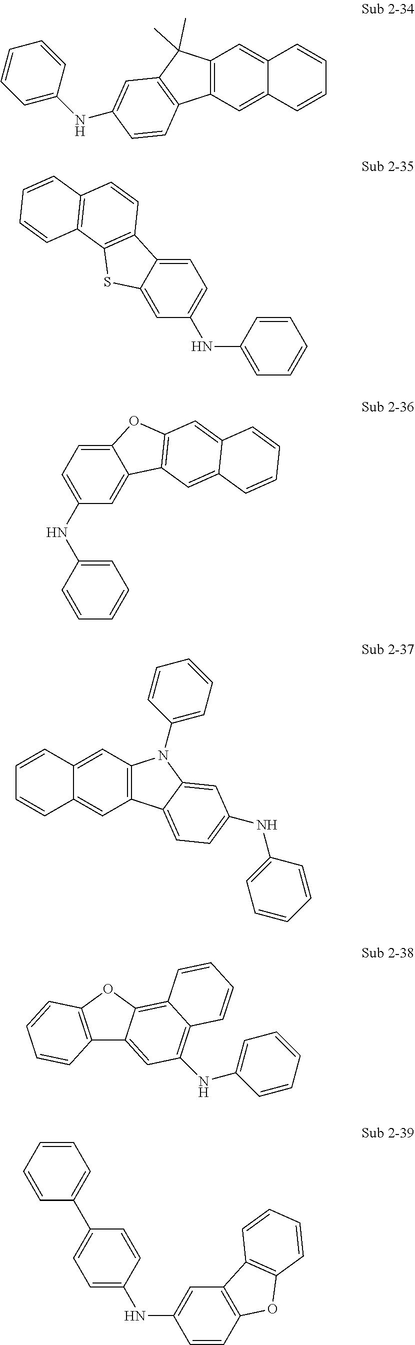 [SMILES: CC1(C)c2cc(Nc3ccccc3)ccc2-c2cc3ccccc3cc21 ; c1ccc(Nc2ccc3c(c2)sc2c4ccccc4ccc32)cc1 ; c1ccc(Nc2ccc3oc4cc5ccccc5cc4c3c2)cc1 ; c1ccc(Nc2ccc3c4cc5ccccc5cc4n(-c4ccccc4)c3c2)cc1 ; c1ccc(Nc2cc3c4ccccc4oc3c3ccccc23)cc1 ; c1ccc(-c2ccc(Nc3ccc4oc5ccccc5c4c3)cc2)cc1]

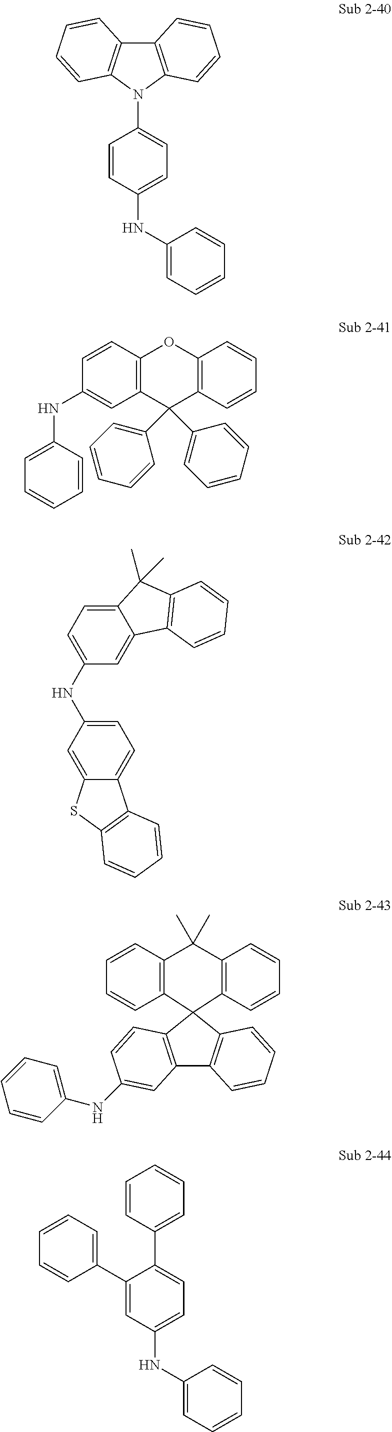 [SMILES: c1ccc(Nc2ccc(-n3c4ccccc4c4ccccc43)cc2)cc1 ; c1ccc(Nc2ccc3c(c2)C(c2ccccc2)(c2ccccc2)c2ccccc2O3)cc1 ; CC1(C)c2ccccc2-c2cc(Nc3ccc4c(c3)sc3ccccc34)ccc21 ; CC1(C)c2ccccc2C2(c3ccccc3-c3cc(Nc4ccccc4)ccc32)c2ccccc21 ; c1ccc(Nc2ccc(-c3ccccc3)c(-c3ccccc3)c2)cc1]

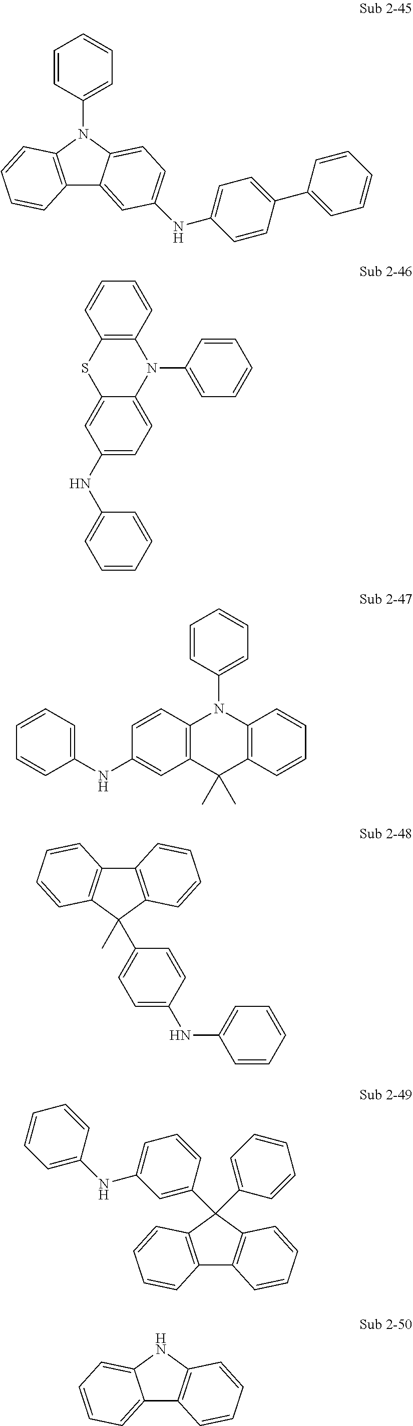 [SMILES: c1ccc(-c2ccc(Nc3ccc4c(c3)c3ccccc3n4-c3ccccc3)cc2)cc1 ; c1ccc(Nc2ccc3c(c2)Sc2ccccc2N3c2ccccc2)cc1 ; CC1(C)c2ccccc2N(c2ccccc2)c2ccc(Nc3ccccc3)cc21 ; CC1(c2ccc(Nc3ccccc3)cc2)c2ccccc2-c2ccccc21 ; c1ccc(Nc2cccc(C3(c4ccccc4)c4ccccc4-c4ccccc43)c2)cc1 ; c1ccc2c(c1)[nH]c1ccccc12]

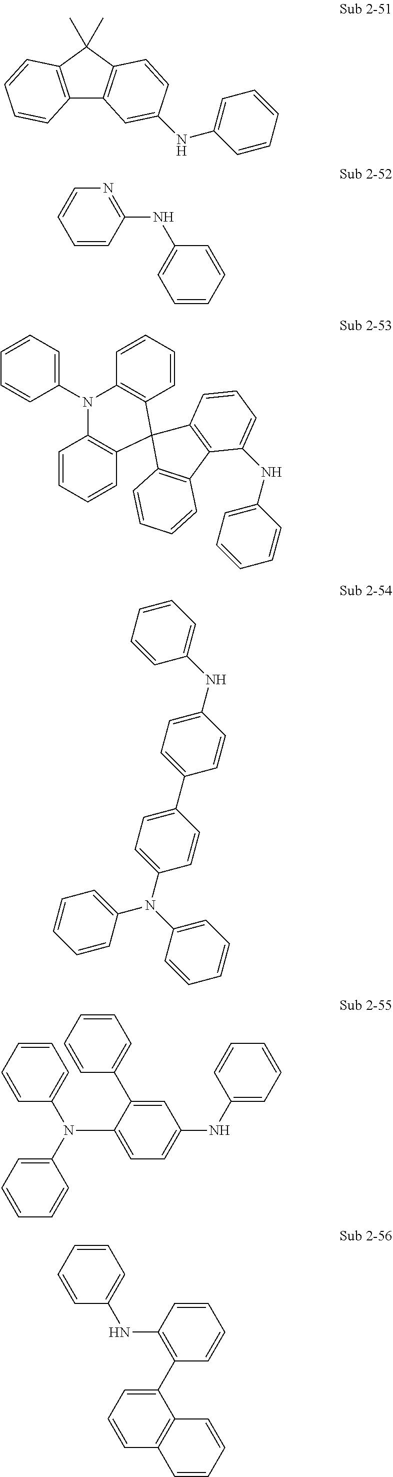

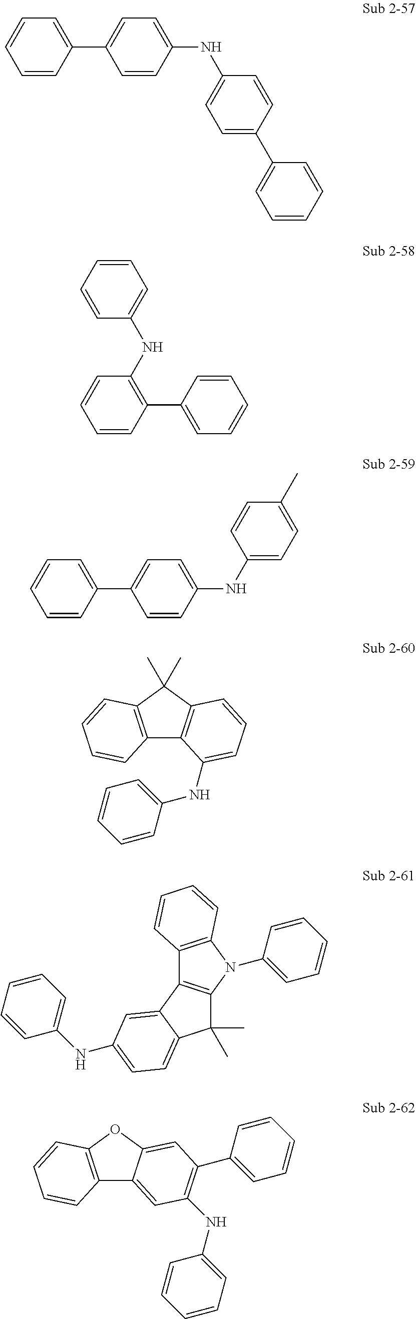

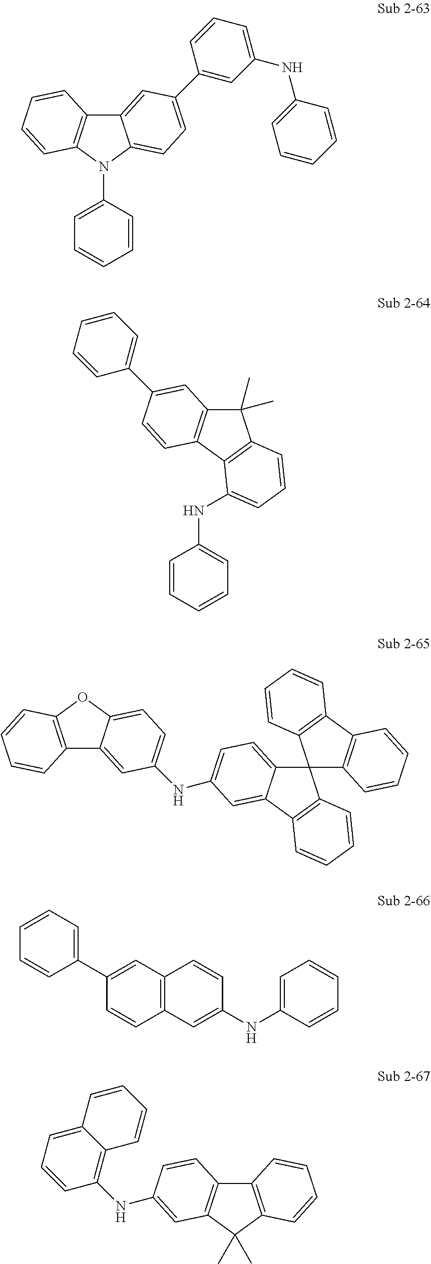 [SMILES: c1ccc(Nc2cccc(-c3ccc4c(c3)c3ccccc3n4-c3ccccc3)c2)cc1 ; CC1(C)c2cc(-c3ccccc3)ccc2-c2c(Nc3ccccc3)cccc21 ; c1ccc2c(c1)-c1ccccc1C21c2ccccc2-c2cc(Nc3ccc4oc5ccccc5c4c3)ccc21 ; c1ccc(Nc2ccc3cc(-c4ccccc4)ccc3c2)cc1 ; CC1(C)c2ccccc2-c2ccc(Nc3cccc4ccccc34)cc21]

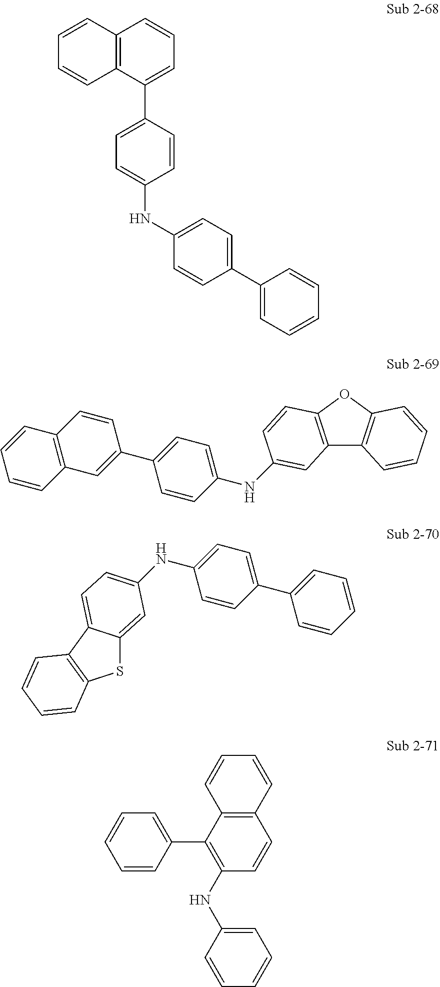 [SMILES: c1ccc(-c2ccc(Nc3ccc(-c4cccc5ccccc45)cc3)cc2)cc1 ; c1ccc2cc(-c3ccc(Nc4ccc5oc6ccccc6c5c4)cc3)ccc2c1 ; c1ccc(-c2ccc(Nc3ccc4c(c3)sc3ccccc34)cc2)cc1 ; c1ccc(Nc2ccc3ccccc3c2-c2ccccc2)cc1]

TABLE 2

| com- | | com- | |
|---|---|---|---|
| pound | FD-MS | pound | FD-MS |
| Sub 2-1 | m/z = 169.09(C₁₂H₁₁N = 169.23) | Sub 2-2 | m/z = 219.1(C₁₆H₁₃N = 219.29) |
| Sub 2-3 | m/z = 245.12(C₁₈H₁₅N = 245.33) | Sub 2-4 | m/z = 285.15(C₂₁H₁₉N = 285.39) |
| Sub 2-5 | m/z = 259.1(C₁₈H₁₃NO = 259.31) | Sub 2-6 | m/z = 269.12(C₂₀H₁₅N = 269.35) |
| Sub 2-7 | m/z = 334.15(C₂₄H₁₈N₂ = 334.42) | Sub 2-8 | m/z = 275.08(C₁₈H₁₃NS = 275.37) |
| Sub 2-9 | m/z = 293.12(C₂₂H₁₅N = 293.37) | Sub 2-10 | m/z = 407.17(C₃₁H₂₁N = 407.52) |
| Sub 2-11 | m/z = 365.09(C₂₄H₁₅NOS = 365.45) | Sub 2-12 | m/z = 259.1(C₁₈H₁₃NO = 259.31) |
| Sub 2-13 | m/z = 295.14(C₂₂H₁₇N = 295.39) | Sub 2-14 | m/z = 269.12(C₂₀H₁₅N = 269.35) |
| Sub 2-15 | m/z = 319.14(C₂₄H₁₇N = 319.41) | Sub 2-16 | m/z = 334.15(C₂₄H₁₈N₂ = 334.42) |
| Sub 2-17 | m/z = 409.18(C₃₁H₂₃N = 409.53) | Sub 2-18 | m/z = 485.21(C₃₇H₂₇N = 485.63) |
| Sub 2-19 | m/z = 351.11(C₂₄H₁₇NS = 351.47) | Sub 2-20 | m/z = 174.12(C₁₂H₆D₅N = 174.26) |
| Sub 2-21 | m/z = 285.15(C₂₁H₁₉N = 285.39) | Sub 2-22 | m/z = 275.09(C₁₈H₁₃NO₂ = 275.31) |
| Sub 2-23 | m/z = 307.05(C₁₈H₁₃NS₂ = 307.43) | Sub 2-24 | m/z = 377.18(C₂₇H₂₃NO = 377.49) |
| Sub 2-25 | m/z = 423.16(C₃₁H₂₁NO = 423.52) | Sub 2-26 | m/z = 299.09(C₂₀H₁₃NO₂ = 299.33) |
| Sub 2-27 | m/z = 301.15(C₂₁H₁₉NO = 301.39) | Sub 2-28 | m/z = 423.16(C₃₁H₂₁NO = 423.52) |
| Sub 2-29 | m/z = 341.12(C₂₃H₁₉NS = 341.47) | Sub 2-30 | m/z = 315.07(C₂₀H₁₃NOS = 315.39) |
| Sub 2-31 | m/z = 317.12(C₂₁H₁₉NS = 317.45) | Sub 2-32 | m/z = 335.17(C₂₅H₂₁N = 335.45) |
| Sub 2-33 | m/z = 309.12(C₂₂H₁₅NO = 309.37) | Sub 2-34 | m/z = 335.17(C₂₅H₂₁N = 335.45) |
| Sub 2-35 | m/z = 325.09(C₂₂H₁₅NS = 325.43) | Sub 2-36 | m/z = 309.12(C₂₂H₁₅NO = 309.37) |
| Sub 2-37 | m/z = 384.16(C₂₈H₂₀N₂ = 384.48) | Sub 2-38 | m/z = 309.12(C₂₂H₁₅NO = 309.37) |
| Sub 2-39 | m/z = 335.13(C₂₄H₁₇NO = 335.41) | Sub 2-40 | m/z = 334.15(C₂₄H₁₈N₂ = 334.42) |
| Sub 2-41 | m/z = 425.18(C₃₁H₂₃NO = 425.53) | Sub 2-42 | m/z = 391.14(C₂₇H₂₁NS = 391.53) |
| Sub 2-43 | m/z = 449.21(C₃₄H₂₇N = 449.6) | Sub 2-44 | m/z = 321.15(C₂₄H₁₉N = 321.42) |
| Sub 2-45 | m/z = 410.18(C₃₀H₂₂N₂ = 410.52) | Sub 2-46 | m/z = 366.12(C₂₄H₁₈N₂S = 366.48) |
| Sub 2-47 | m/z = 376.19(C_{27l H24}N₂ = 376.5) | Sub 2-48 | m/z = 347.17(C₂₆H₂₁N = 347.46) |
| Sub 2-49 | m/z = 409.18(C₃₁H₂₃N = 409.53) | Sub 2-50 | m/z = 167.07(C₁₂H₉N = 167.21) |
| Sub 2-51 | m/z = 285.15(C₂₁H₁₉N = 285.39) | Sub 2-52 | m/z = 170.08(C₁₁H₁₀N₂ = 170.22) |
| Sub 2-53 | m/z = 498.21(C₃₇H₂₆N₂ = 498.63) | Sub 2-54 | m/z = 412.19(C₃₀H₂₄N₂ = 412.54) |
| Sub 2-55 | m/z = 412.19(C₃₀H₂₄N₂ = 412.54) | Sub 2-56 | m/z = 295.14(C₂₂H₁₇N = 295.39) |
| Sub 2-57 | m/z = 321.15(C₂₄H₁₉N = 321.42) | Sub 2-58 | m/z = 245.12(C₁₈H₁₅N = 245.33) |
| Sub 2-59 | m/z = 259.14(C₁₉H₁₇N = 259.35) | Sub 2-60 | m/z = 285.15(C₂₁H₁₉N = 285.39) |
| Sub 2-61 | m/z = 400.19(C₂₉H₂₄N₂ = 400.53) | Sub 2-62 | m/z = 335.13(C₂₄H₁₇NO = 335.41) |
| Sub 2-63 | m/z = 410.18(C₃₀H₂₂N₂ = 410.52) | Sub 2-64 | m/z = 361.18(C₂₇H₂₃N = 361.49) |
| Sub 2-65 | m/z = 497.18(C₃₇H₂₃NO = 497.6) | Sub 2-66 | m/z = 295.14(C₂₂H₁₇N = 295.39) |
| Sub 2-67 | m/z = 335.17(C₂₅H₂₁N = 335.45) | Sub 2-68 | m/z = 371.17(C₂₈H₂₁N = 371.48) |
| Sub 2-69 | m/z = 385.15(C₂₈H₁₉NO = 385.47) | Sub 2-70 | m/z = 351.11(C₂₄H₁₇NS = 351.47) |
| Sub 2-71 | m/z = 295.14(C₂₂H₁₇N = 295.39) | | |

### Synthesis Example of Final Products

### 1. Synthesis Example of 1-5

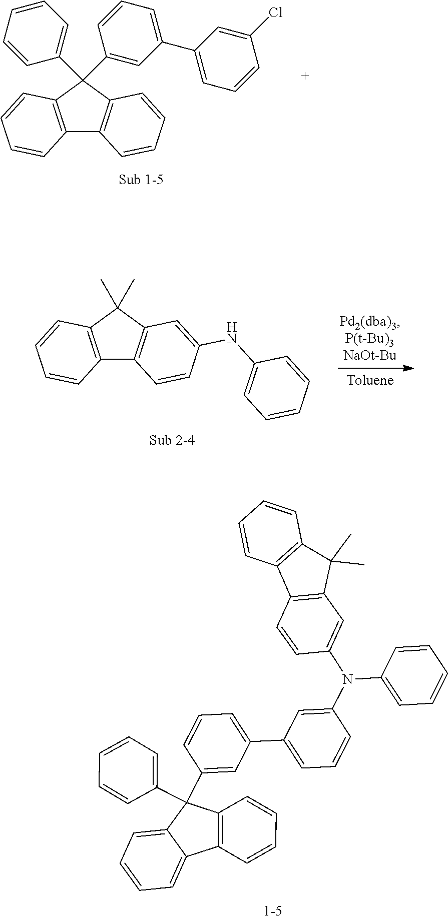 [SMILES: Clc1cccc(-c2cccc(C3(c4ccccc4)c4ccccc4-c4ccccc43)c2)c1 ; CC1(C)c2ccccc2-c2ccc(Nc3ccccc3)cc21 ; CC1(C)c2ccccc2-c2ccc(N(c3ccccc3)c3cccc(-c4cccc(C5(c6ccccc6)c6ccccc6-c6ccccc65)c4)c3)cc21]

Sub 2-4 (13.3 g, 46.6 mmol), Pd₂(dba)₃ (1.28 g, 1.4 mmol), P(t-Bu)₃ (0.57 g, 2.8 mmol), NaOt-Bu (9.0 g, 93.2 mmol), Toluene (200 mL) were added to Sub 1-5 (20 g, 46.6 mmol) and reacted for 2 hours. When the reaction is complete, the mixture is extracted with water, the organic layer is dried over MgSO₄ and concentrated, and the resulting compound was recrystallized by silicagel column to obtain 1-5 (22.4 g, yield: 71%).

### 2. Synthesis Example of 1-9

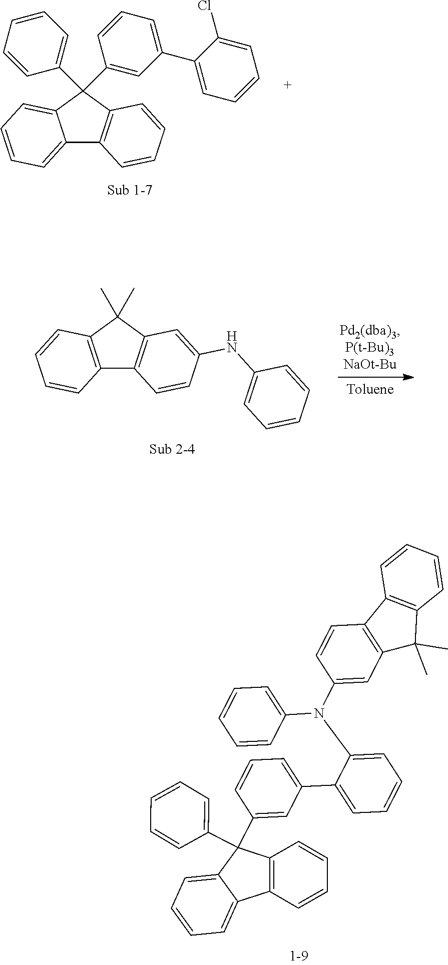 [SMILES: Clc1ccccc1-c1cccc(C2(c3ccccc3)c3ccccc3-c3ccccc32)c1 ; CC1(C)c2ccccc2-c2ccc(Nc3ccccc3)cc21 ; CC1(C)c2ccccc2-c2ccc(N(c3ccccc3)c3ccccc3-c3cccc(C4(c5ccccc5)c5ccccc5-c5ccccc54)c3)cc21]

Sub 2-4 (13.3 g, 46.6 mmol), Pd₂(dba)₃ (1.28 g, 1.4 mmol), P(t-Bu)₃ (0.57 g, 2.8 mmol), NaOt-Bu (9.0 g, 93.2 mmol), Toluene (200 mL) were added to Sub 1-7 (20 g, 46.6 mmol) and reacted for 2 hours. When the reaction is complete, the mixture is extracted with water, the organic layer is dried over MgSO₄ and concentrated, and the resulting compound was recrystallized by silicagel column to obtain 1-9 (21.8 g, yield: 69%).

### 3. Synthesis Example of 1-32

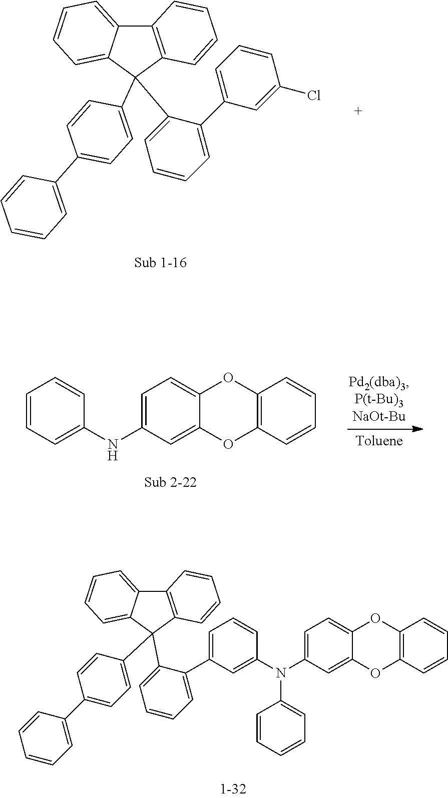 [SMILES: Clc1cccc(-c2ccccc2C2(c3ccc(-c4ccccc4)cc3)c3ccccc3-c3ccccc32)c1 ; c1ccc(Nc2ccc3c(c2)Oc2ccccc2O3)cc1 ; c1ccc(-c2ccc(C3(c4ccccc4-c4cccc(N(c5ccccc5)c5ccc6c(c5)Oc5ccccc5O6)c4)c4ccccc4-c4ccccc43)cc2)cc1]

Sub 2-22 (10.9 g, 39.6 mmol), Pd₂(dba)₃ (1.09 g, 1.19 mmol), P(t-Bu)₃ (0.48 g, 2.38 mmol), NaOt-Bu (7.6 g, 79.2 mmol), Toluene (200 mL) were added to Sub 1-16 (20 g, 39.6 mmol) and reacted for 2 hours. When the reaction is complete, the mixture is extracted with water, the organic layer is dried over MgSO₄ and concentrated, and the resulting compound was recrystallized by silicagel column to obtain 1-32 (18.3 g, yield: 62%).

### 4. Synthesis Example of 1-56

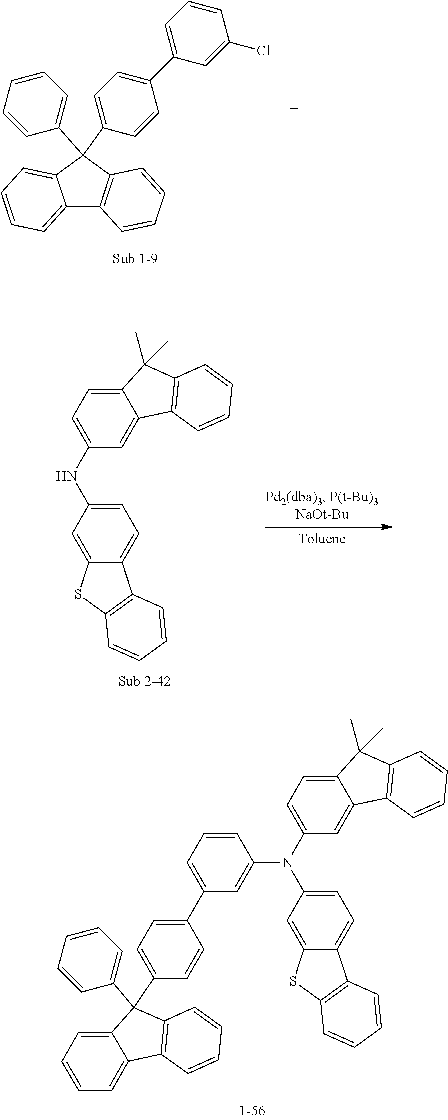 [SMILES: Clc1cccc(-c2ccc(C3(c4ccccc4)c4ccccc4-c4ccccc43)cc2)c1 ; CC1(C)c2ccccc2-c2cc(Nc3ccc4c(c3)sc3ccccc34)ccc21 ; CC1(C)c2ccccc2-c2cc(N(c3cccc(-c4ccc(C5(c6ccccc6)c6ccccc6-c6ccccc65)cc4)c3)c3ccc4c(c3)sc3ccccc34)ccc21]

Sub 2-42 (18.3 g, 46.6 mmol), Pd₂(dba)₃ (1.28 g, 1.4 mmol), P(t-Bu)₃ (0.57 g, 2.8 mmol), NaOt-Bu (9.0 g, 93.2 mmol), Toluene (230 mL) were added to Sub 1-9 (20 g, 46.6 mmol) and reacted for 2 hours. When the reaction is complete, the mixture is extracted with water, the organic layer is dried over MgSO₄ and concentrated, and the resulting compound was recrystallized by silicagel column to obtain 1-56 (25.6 g, yield: 70%).

### 5. Synthesis Example of 1-57

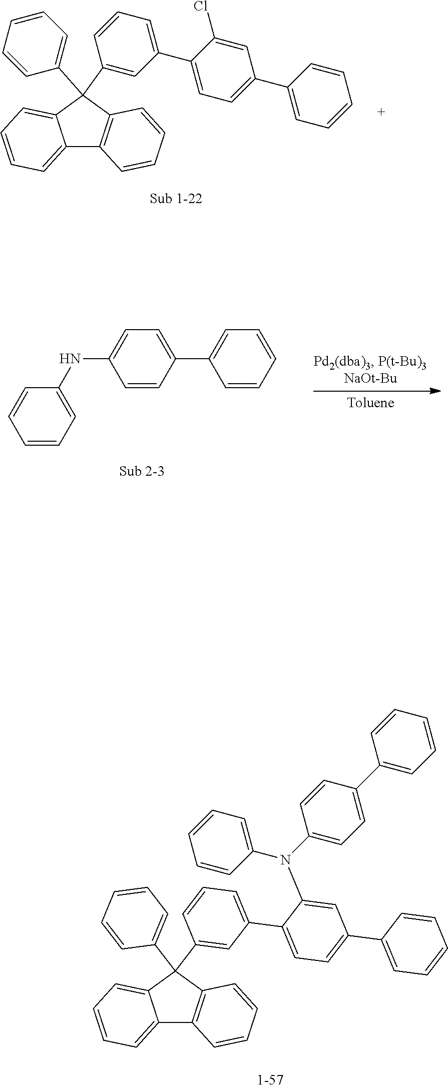 [SMILES: Clc1cc(-c2ccccc2)ccc1-c1cccc(C2(c3ccccc3)c3ccccc3-c3ccccc32)c1 ; c1ccc(Nc2ccc(-c3ccccc3)cc2)cc1 ; c1ccc(-c2ccc(N(c3ccccc3)c3cc(-c4ccccc4)ccc3-c3cccc(C4(c5ccccc5)c5ccccc5-c5ccccc54)c3)cc2)cc1]

Sub 2-3 (9.7 g, 39.6 mmol), Pd₂(dba)₃ (1.09 g, 1.19 mmol), P(t-Bu)₃ (0.48 g, 2.38 mmol), NaOt-Bu (7.6 g, 79.2 mmol), Toluene (200 mL) were added to Sub 1-22 (20 g, 39.6 mmol) and reacted for 2 hours. When the reaction is complete, the mixture is extracted with water, the organic layer is dried over MgSO₄ and concentrated, and the resulting compound was recrystallized by silicagel column to obtain 1-57 (18.9 g, yield: 67%).

### 6. Synthesis Example of 1-75

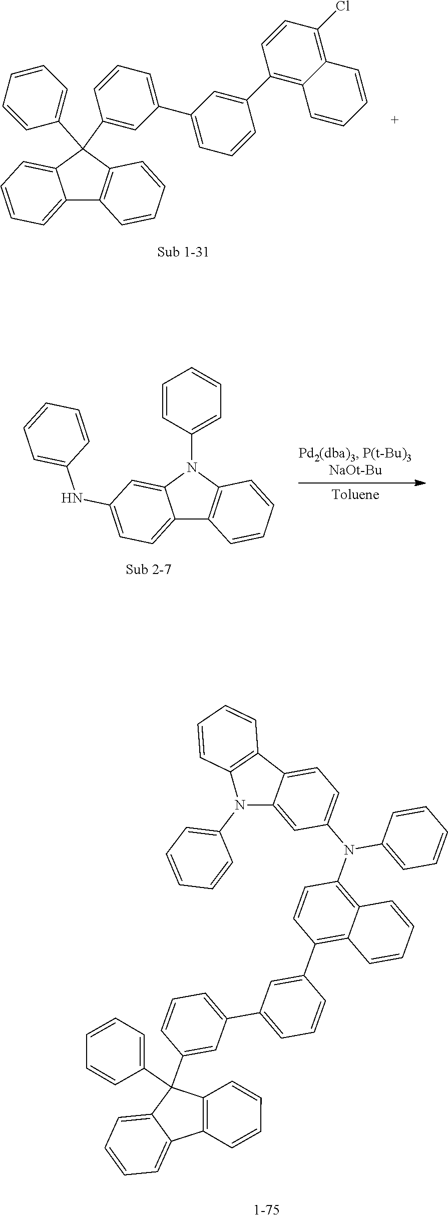 [SMILES: Clc1ccc(-c2cccc(-c3cccc(C4(c5ccccc5)c5ccccc5-c5ccccc54)c3)c2)c2ccccc12 ; c1ccc(Nc2ccc3c4ccccc4n(-c4ccccc4)c3c2)cc1 ; c1ccc(N(c2ccc3c4ccccc4n(-c4ccccc4)c3c2)c2ccc(-c3cccc(-c4cccc(C5(c6ccccc6)c6ccccc6-c6ccccc65)c4)c3)c3ccccc23)cc1]

Sub 2-7 (12.0 g, 36.0 mmol), Pd₂(dba)₃ (0.99 g, 1.08 mmol), P(t-Bu)₃ (0.44 g, 2.16 mmol), NaOt-Bu (6.9 g, 72.1 mmol), Toluene (200 mL) were added to Sub 1-31 (20 g, 36.0 mmol) and reacted for 2 hours. When the reaction is complete, the mixture is extracted with water, the organic layer is dried over MgSO₄ and concentrated, and the resulting compound was recrystallized by silicagel column to obtain 1-75 (21.5 g, yield: 70%).

### 7. Synthesis Example of 1-82

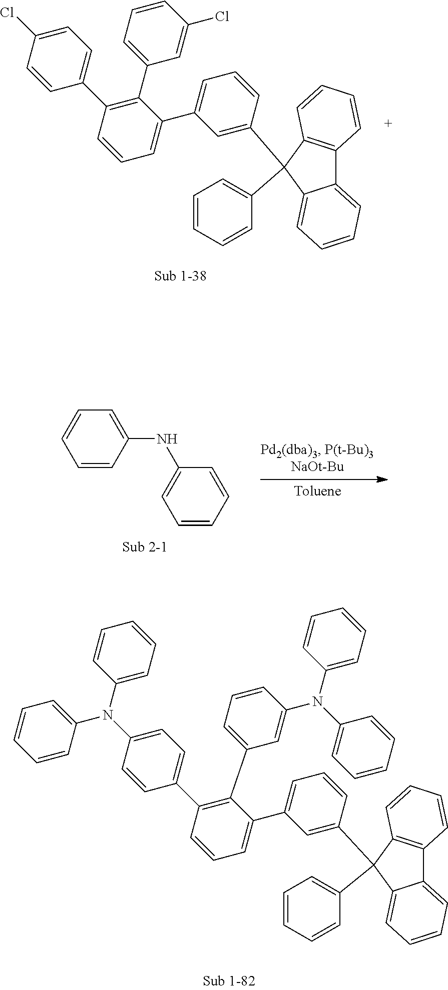 [SMILES: Clc1ccc(-c2cccc(-c3cccc(C4(c5ccccc5)c5ccccc5-c5ccccc54)c3)c2-c2cccc(Cl)c2)cc1 ; c1ccc(Nc2ccccc2)cc1 ; c1ccc(N(c2ccccc2)c2ccc(-c3cccc(-c4cccc(C5(c6ccccc6)c6ccccc6-c6ccccc65)c4)c3-c3cccc(N(c4ccccc4)c4ccccc4)c3)cc2)cc1]

Sub 2-1 (11.0 g, 65.0 mmol), Pd₂(dba)₃ (1.78 g, 1.95 mmol), P(t-Bu)₃ (0.79 g, 3.9 mmol), NaOt-Bu (12.5 g, 130.0 mmol), Toluene (180 mL) were added to Sub 1-38 (20 g, 32.5 mmol) and reacted for 2 hours. When the reaction is complete, the mixture is extracted with water, the organic layer is dried over MgSO₄ and concentrated, and the resulting compound was recrystallized by silicagel column to obtain 1-82 (19.5 g, yield: 68%).

### 8. Synthesis Example of 1-109

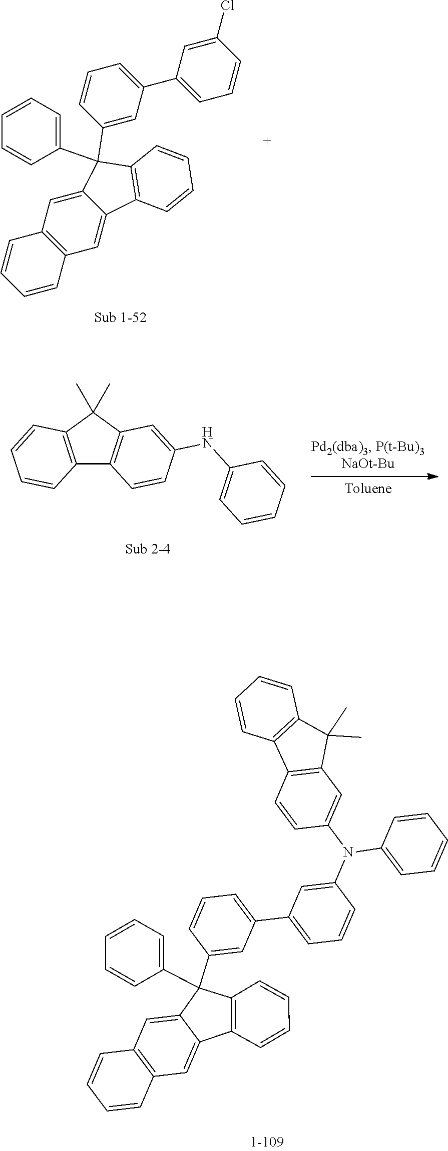 [SMILES: Clc1cccc(-c2cccc(C3(c4ccccc4)c4ccccc4-c4cc5ccccc5cc43)c2)c1 ; CC1(C)c2ccccc2-c2ccc(Nc3ccccc3)cc21 ; CC1(C)c2ccccc2-c2ccc(N(c3ccccc3)c3cccc(-c4cccc(C5(c6ccccc6)c6ccccc6-c6cc7ccccc7cc65)c4)c3)cc21]

Sub 2-4 (11.9 g, 41.8 mmol), Pd₂(dba)₃ (1.15 g, 1.25 mmol), P(t-Bu)₃ (0.51 g, 2.51 mmol), NaOt-Bu (8.0 g, 83.5 mmol), Toluene (200 mL) were added to Sub 1-52 (20 g, 41.8 mmol) and reacted for 2 hours. When the reaction is complete, the mixture is extracted with water, the organic layer is dried over MgSO₄ and concentrated, and the resulting compound was recrystallized by silicagel column to obtain 1-109 (21.9 g, yield: 72%).

### 9. Synthesis Example of 1-115

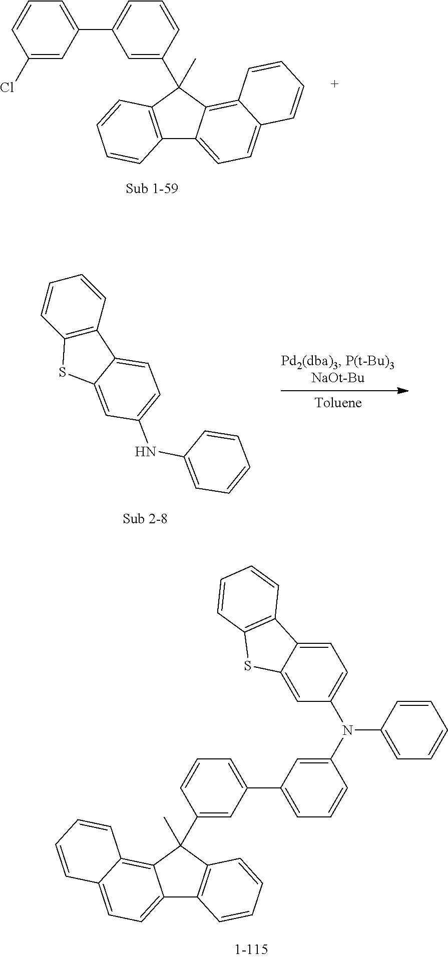 [SMILES: CC1(c2cccc(-c3cccc(Cl)c3)c2)c2ccccc2-c2ccc3ccccc3c21 ; c1ccc(Nc2ccc3c(c2)sc2ccccc23)cc1 ; CC1(c2cccc(-c3cccc(N(c4ccccc4)c4ccc5c(c4)sc4ccccc45)c3)c2)c2ccccc2-c2ccc3ccccc3c21]

Sub 2-8 (13.2 g, 48.0 mmol), Pd₂(dba)₃ (1.32 g, 1.44 mmol), P(t-Bu)₃ (0.58 g, 2.88 mmol), NaOt-Bu (9.2 g, 95.9 mmol), Toluene (240 mL) were added to Sub 1-59 (20 g, 48.0 mmol) and reacted for 2 hours. When the reaction is complete, the mixture is extracted with water, the organic layer is dried over MgSO₄ and concentrated, and the resulting compound was recrystallized by silicagel column to obtain 1-115 (22.0 g, yield: 70%).

Meanwhile, FD-MS values of compounds 1-1 to 1-132 of the present invention prepared according to the above synthesis examples are shown in Table 3.

TABLE 3

| com- | | com- | |
|---|---|---|---|
| pound | FD-MS | pound | FD-MS |
| 1-1 | m/z = 499.23(C₃₈H₂₉N = 499.66) | 1-2 | m/z = 499.23(C₃₈H₂₉N = 499.66) |
| 1-3 | m/z = 549.25(C₄₂H₃₁N = 549.72) | 1-4 | m/z = 637.28(C₄₉H₃₅N = 637.83) |
| 1-5 | m/z = 677.31(C₅₂H₃₉N = 677.89) | 1-6 | m/z = 561.25(C₄₃H₃₁N = 561.73) |
| 1-7 | m/z = 651.26(C₄₉H₃₃NO = 651.81) | 1-8 | m/z = 661.28(C₅₁H₃₅N = 661.85) |
| 1-9 | m/z = 677.31(C₅₂H₃₉N = 677.89) | 1-10 | m/z = 664.29(C₅₀H₃₆N₂ = 664.8) |
| 1-11 | m/z = 605.22(C₄₄H₃₁NS = 605.8) | 1-12 | m/z = 685.28(C₅₃H₃₅N = 685.87) |
| 1-13 | m/z = 799.32(C₆₂H₄₁N = 800.02) | 1-14 | m/z = 687.29(C₅₃H₃₇N = 687.89) |
| 1-15 | m/z = 757.24(C₅₅H₃₅NOS = 757.95) | 1-16 | m/z = 726.3(C₅₅H₃₈N₂ = 726.92) |
| 1-17 | m/z = 589.24(C₄₄H₃₁NO = 589.74) | 1-18 | m/z = 687.29(C₅₃H₃₇N = 687.89) |
| 1-19 | m/z = 711.29(C₅₅H₃₇N = 711.91) | 1-20 | m/z = 711.29(C₅₅H₃₇N = 711.91) |
| 1-21 | m/z = 739.32(C₅₇H₄₁N = 739.96) | 1-22 | m/z = 677.31(C₅₂H₃₉N = 677.89) |
| 1-23 | m/z = 611.26(C₄₇H₃₃N = 611.79) | 1-24 | m/z = 726.3(C₅₅H₃₈N₂ = 726.92) |
| 1-25 | m/z = 677.31(C₅₂H₃₉N = 677.89) | 1-26 | m/z = 815.36(C₆₃H₄₅N = 816.06) |
| 1-27 | m/z = 819.30(C₆₁H₄₁NS = 820.07) | 1-28 | m/z = 637.28(C₄₉H₃₅N = 637.83) |
| 1-29 | m/z = 566.28(C₄₃H₂₆D₅N = 566.76) | 1-30 | m/z = 681.33(C₅₂H₃₅D₄N = 681.92) |
| 1-31 | m/z = 586.24(C₄₄H₃₀N₂ = 586.74) | 1-32 | m/z = 743.28(C₅₅H₃₇NO₂ = 743.91) |
| 1-33 | m/z = 817.31(C₆₂H₄₀FN = 818.01) | 1-34 | m/z = 699.21(C₄₉H₃₃NS₂ = 699.93) |
| 1-35 | m/z = 769.33(C₅₈H₄₃NO = 769.99) | 1-36 | m/z = 651.26(C₄₉H₃₃NO = 651.81) |
| 1-37 | m/z = 617.31(C₄₇H₃₉N = 617.84) | 1-38 | m/z = 691.25(C₅₁H₃₃NO₂ = 691.83) |
| 1-39 | m/z = 693.3(C₅₂H₃₉NO = 693.89) | 1-40 | m/z = 815.32(C₆₂H₄₁NO = 816.02) |
| 1-41 | m/z = 815.32(C₆₂H₄₁NO = 816.02) | 1-42 | m/z = 671.26(C₄₉H₃₇NS = 671.9) |
| 1-43 | m/z = 707.23(C₅₁H₃₃NOS = 707.89) | 1-44 | m/z = 785.31(C₅₈H₄₃NS = 786.05) |
| 1-45 | m/z = 727.32(C₅₆H₄₁N = 727.95) | 1-46 | m/z = 727.32(C₅₆H₄₁N = 727.95) |
| 1-47 | m/z = 655.23(C₄₈H₃₃NS = 655.86) | 1-48 | m/z = 701.27(C₅₃H₃₅NO = 701.87) |
| 1-49 | m/z = 677.31(C₅₂H₃₉N = 677.89) | 1-50 | m/z = 701.27(C₅₃H₃₅NO = 701.87) |
| 1-51 | m/z = 776.32(C₅₉H₄₀N₂ = 776.98) | 1-52 | m/z = 701.27(C₅₃H₃₅NO = 701.87) |
| 1-53 | m/z = 677.31(C₅₂H₃₉N = 677.89) | 1-54 | m/z = 665.27(C₅₀H₃₅NO = 665.84) |
| 1-55 | m/z = 726.3(C₅₅H₃₈N₂ = 726.92) | 1-56 | m/z = 783.3(C₅₈H₄₁NS = 784.03) |
| 1-57 | m/z = 713.31(C₅₅H₃₉N = 713.92) | 1-58 | m/z = 727.32(C₅₆H₄₁N = 727.95) |
| 1-59 | m/z = 817.33(C₆₂H₄₃NO = 818.03) | 1-60 | m/z = 611.26(C₄₇H₃₃N = 611.79) |
| 1-61 | m/z = 841.37(C₆₅H₄₇N = 842.1) | 1-62 | m/z = 713.31(C₅₅H₃₉N = 713.92) |
| 1-63 | m/z = 651.26(C₄₉H₃₃NO = 651.81) | 1-64 | m/z = 802.33(C₆₁H₄₂N₂ = 803.02) |
| 1-65 | m/z = 829.33(C₆₃H₄₃NO = 830.04) | 1-66 | m/z = 758.28(C₅₅H₃₈N₂S = 758.98) |
| 1-67 | m/z = 768.35(C₅₈H₄₄N₂ = 769) | 1-68 | m/z = 716.29(C₅₂H₃₆N₄ = 716.89) |
| 1-69 | m/z = 801.34(C₆₂H₄₃N = 802.03) | 1-70 | m/z = 559.23(C₄₃H₂₉N = 559.71) |
| 1-71 | m/z = 739.32(C₅₇H₄₁N = 739.96) | 1-72 | m/z = 637.28(C₄₉H₃₅N = 637.83) |
| 1-73 | m/z = 753.34(C₅₈H₄₃N = 753.99) | 1-74 | m/z = 638.27(C₄₈H₃₄N₂ = 638.81) |
| 1-75 | m/z = 852.35(C₆₅H₄₄N₂ = 853.08) | 1-76 | m/z = 777.3(C₅₉H₃₉NO = 777.97) |
| 1-77 | m/z = 687.29(C₅₃H₃₇N = 687.89) | 1-78 | m/z = 713.31(C₅₅H₃₉N = 713.92) |
| 1-79 | m/z = 744.26(C₅₄H₃₆N₂S = 744.96) | 1-80 | m/z = 804.35(C₆₁H₄₄N₂ = 805.04) |
| 1-81 | m/z = 970.43(C₇₄H₅₄N₂= 971.26) | 1-82 | m/z = 880.38(C₆₇H₄₈N₂ = 881.14) |
| 1-83 | m/z = 894.36(C₆₇H₄₆N₂O = 895.12) | 1-84 | m/z = 804.35(C₆₁H₄₄N₂ = 805.04) |
| 1-85 | m/z = 804.35(C₆₁H₄₄N₂ = 805.04) | 1-86 | m/z = 804.35(C₆₁H₄₄N₂ = 805.04) |
| 1-87 | m/z = 804.35(C₆₁H₄₄N₂ = 805.04) | 1-88 | m/z = 854.37(C₆₅H₄₆N₂ = 855.1) |
| 1-89 | m/z = 828.35(C₆₃H₄₄N₂ = 829.06) | 1-90 | m/z = 763.32(C₅₉H₄₁N = 763.98) |
| 1-91 | m/z = 713.31(C₅₅H₃₉N = 713.92) | 1-92 | m/z = 637.28(C₄₉H₃₅N = 637.8) |
| 1-93 | m/z = 615.29(C₄₇H₃₇N = 615.82) | 1-94 | m/z = 687.29(C₅₃H₃₇N = 687.89) |
| 1-95 | m/z = 651.29(C₅₀H₃₇N = 651.85) | 1-96 | m/z = 656.26(C₄₈H₃₃FN₂ = 656.8) |
| 1-97 | m/z = 803.36(C₆₂H₄₅N = 804.05) | 1-98 | m/z = 792.35(C₆₀H₄₄N₂ = 793.03) |
| 1-99 | m/z = 802.33(C₆₁H₄₂N₂ = 803.02) | 1-100 | m/z = 727.29(C₅₅H₃₇NO = 727.91) |
| 1-101 | m/z = 829.37(C₆₄H₄₇N = 830.09) | 1-102 | m/z = 753.34(C₅₈H₄₃N = 753.99) |
| 1-103 | m/z = 802.33(C₆₁H₄₂N₂ = 803.02) | 1-104 | m/z = 889.33(C₆₈H₄₃NO = 890.1) |
| 1-105 | m/z = 549.25(C₄₂H₃₁N = 549.72) | 1-106 | m/z = 549.25(C₄₂H₃₁N = 549.72) |
| 1-107 | m/z = 599.26(C₄₆H₃₃N = 599.78) | 1-108 | m/z = 687.29(C₅₃H₃₇N = 687.89) |
| 1-109 | m/z = 727.32(C₅₆H₄₁N = 727.95) | 1-110 | m/z = 611.26(C₄₇H₃₃N = 611.79) |
| 1-111 | m/z = 701.27(C₅₃H₃₅NO = 701.87) | 1-112 | m/z = 711.29(C₅₅H₃₇N = 711.91) |
| 1-113 | m/z = 727.32(C₅₆H₄₁N = 727.95) | 1-114 | m/z = 714.3(C₅₄H₃₈N₂ = 714.91) |
| 1-115 | m/z = 655.23(C₄₈H₃₃NS = 655.86) | 1-116 | m/z = 737.31(C₅₇H₃₉N = 737.95) |
| 1-117 | m/z = 616.29(C₄₇H₂₈D₅N = 616.82) | 1-118 | m/z = 731.35(C₅₆H₃₇D₄N = 731.98) |
| 1-119 | m/z = 636.26(C₄₈H₃₂N₂ = 636.8) | 1-120 | m/z = 793.3(C₅₉H₃₉NO₂ = 793.97) |
| 1-121 | m/z = 875.36(C₆₈H₄₅N = 876.12) | 1-122 | m/z = 775.24(C₅₅H₃₇NS₂ = 776.03) |
| 1-123 | m/z = 825.4(C₆₂H₅₁NO = 826.1) | 1-124 | m/z = 803.36(C₆₂H₄₅N = 804.05) |
| 1-125 | m/z = 877.37(C₆₈H₄₇N = 878.13) | 1-126 | m/z = 803.36(C₆₂H₄₅N = 804.05) |
| 1-127 | m/z = 789.34(C₆₁H₄₃N = 790.02) | 1-128 | m/z = 839.36(C₆₅H₄₅N = 840.08) |
| 1-129 | m/z = 829.37(C₆₄H₄₇N = 830.09) | 1-130 | m/z = 715.29(C₅₄H₃₇NO = 715.9) |
| 1-131 | m/z = 819.3(C₆₁H₄₁NS = 820.07) | 1-132 | m/z = 687.29(C₅₃H₃₇N = 687.89) |

### Manufacturing Evaluation of Organic Electronic Elements

### [Example 1] Green Organic Light Emitting Device (Hole Transport Layer)

After vacuum deposition of N1-(naphthalen-2-yl)-N4,N4-bis(4-(naphthalen-2-yl(phenyl)amino)phenyl)-N1-phenylbenzene-1,4-diamine(Hereinafter, abbreviated as 2-TNATA) to a thickness of 60 nm on the ITO layer (anode) formed on the glass substrate to form a hole injection layer, Compound 1-5 of the present invention was vacuum-deposited on the hole injection layer to a thickness of 60 nm to form a hole transport layer.

Then, an emitting layer having a thickness of 30 nm was formed on the hole transport layer by using 4,4′-N,N′-dicarbazolebiphenyl (hereinafter abbreviated as CBP) as a host and Ir(ppy)3 as a dopant in a weight ratio of 90:10. Then, (1,1′-biphenyl-4-olato)bis(2-methyl-8-quinolinolato)aluminum (hereinafter abbreviated as BAlq) was deposited on the emitting layer to a thickness of 10 nm to form a hole blocking layer, bis(10-hydroxybenzo[h]quinolinato)beryllium (hereinafter abbreviated as BeBq2) was deposited on the hole blocking layer to a thickness of 35 nm to form an electron transport layer. Thereafter, LiF was deposited to a thickness of 0.2 nm to form an electron injection layer, and then Al was deposited to a thickness of 150 nm to form a cathode, thereby manufacturing an organic electroluminescent device.

### [Example 2] to [Example 10]

An organic electroluminescent device was manufactured in the same manner as in Example 1, except that the compound of the present invention described in Table 4 was used instead of Compound 1-5 as the material of the hole transport layer.

### [Comparative Example 2] to [Comparative Example 10]

An organic electroluminescent device was manufactured in the same manner as in Example 1, except that the following comparative compound A or B was used instead of the compound 1-5 of the present invention as a material for the hole transport layer.

<comparative compound A> <comparative compound A>

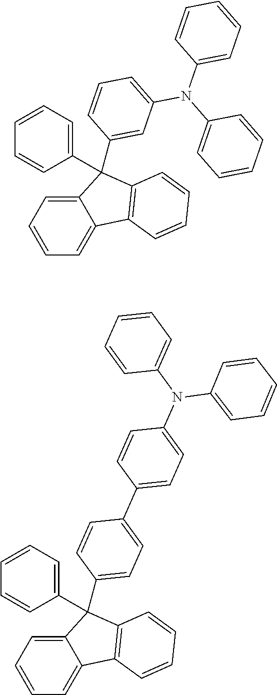 [SMILES: c1ccc(N(c2ccccc2)c2cccc(C3(c4ccccc4)c4ccccc4-c4ccccc43)c2)cc1 ; c1ccc(N(c2ccccc2)c2ccc(-c3ccc(C4(c5ccccc5)c5ccccc5-c5ccccc54)cc3)cc2)cc1]

By applying a forward bias DC voltage to the organic electroluminescent devices manufactured by Examples 1 to 10, Comparative Examples 1 and 2 of the present invention, Electroluminescence (EL) characteristics were measured with PR-650 from Photoresearch, and as a result of the measurement, the T95 lifetime was measured using a lifetime measuring device manufactured by McScience at 5000 cd/m² standard luminance. Table 4 below shows the device fabrication and evaluation results.

TABLE 4

| | | | Current | | | | | |
|---|---|---|---|---|---|---|---|---|
| | | | Density | Brightness | Efficiency | | CIE | |
| | compound | Voltage | (mA/cm²) | (cd/m²) | (cd/A) | T(95) | X | Y |
| comparative | comparative | 6.3 | 19.3 | 5000 | 25.9 | 87.1 | 0.31 | 0.61 |
| example(1) | compound A | | | | | | | |
| comparative | comparative | 6.1 | 15.9 | 5000 | 31.4 | 94.5 | 0.30 | 0.62 |
| example(2) | compound B | | | | | | | |
| example(1) | 1-5 | 5.3 | 12.0 | 5000 | 41.8 | 119.3 | 0.33 | 0.61 |
| example(2) | 1-9 | 5.3 | 11.9 | 5000 | 42.0 | 118.8 | 0.33 | 0.60 |
| example(3) | 1-15 | 5.4 | 12.4 | 5000 | 40.2 | 115.4 | 0.31 | 0.62 |
| example(4) | 1-24 | 5.4 | 12.2 | 5000 | 41.1 | 117.2 | 0.33 | 0.61 |
| example(5) | 1-32 | 5.4 | 12.4 | 5000 | 40.4 | 119.5 | 0.30 | 0.60 |
| example(6) | 1-82 | 5.4 | 12.9 | 5000 | 38.7 | 106.4 | 0.33 | 0.60 |
| example(7) | 1-88 | 5.5 | 12.7 | 5000 | 39.3 | 107.7 | 0.30 | 0.61 |
| example(8) | 1-109 | 5.1 | 13.2 | 5000 | 38.0 | 110.9 | 0.32 | 0.61 |
| example(9) | 1-113 | 5.1 | 13.0 | 5000 | 38.5 | 110.8 | 0.32 | 0.62 |
| example(10) | 1-115 | 5.2 | 13.3 | 5000 | 37.6 | 109.6 | 0.31 | 0.61 |

As can be seen from the results in Table 4, when manufacturing a green organic electronic element using the material for an organic electronic element of the present invention as an hole transport layer material, Compared to Comparative Examples 1 and 2 in which Comparative Compounds A and B were used as the hole transport layer material, the driving voltage of the organic electroluminescent device could be lowered, and the luminous efficiency and lifespan were significantly improved. More specifically, compared to Comparative Example 1 in which a hole transport layer was formed using Comparative Compound A in which phenylene was substituted between the fluorene moiety and the amino group, the device result of Comparative Example 2 in which a hole transport layer was formed using Comparative Compound B in which biphenylene was substituted between a fluorene moiety and an amino group similarly to the compound of the present invention showed an improved value.

The compound of the present invention and Comparative Compound B are identical in that a biphenylene moiety is substituted between the fluorene moiety and the amino group, but the compound of the present invention is different in that the fluorene moiety and the amino group are substituted at the ortho or meta position based on the benzene ring where the amino group is substituted among the biphenylene moieties.

Accordingly, looking at the device results of Comparative Examples and Examples, although Comparative Compounds A and B and the compounds of the present invention consist of similar components, it can be seen that the physical properties of the compound are significantly different depending on the structure of the arylene group between the fluorene moiety and the amino group and the substitution position of the arylene group, when viewed based on the benzene ring substituted with the amino group in the biphenylene moiety as in the compound of the present invention, by substituting the fluorene moiety and the amino group at the ortho or meta position, the physical properties of the compound, such as hole properties, light efficiency properties, energy levels (LUMO and HOMO level, T1 level) hole injection & mobility properties, and electron blocking properties, are more suitable for the hole transport layer, as a result, it can be seen that the device results of Examples 1 to 10, which are completely different from the device characteristics of Comparative Examples 1 and 2, can be derived.

In addition, in the evaluation results of the above-described device fabrication, the device characteristics were described in which the compound of the present invention was applied to the hole transport layer, the compound of the present invention may be applied to any one or more layers of an emitting layer, a hole transport layer, an emitting auxiliary layer, and a capping layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long lifespan, and thus there is industrial applicability.

What is claimed is:

1. A compound represented by Formula (1): 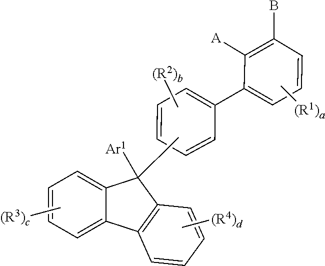
wherein:
1) R¹, R², R³, R⁴, A and B are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₂-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or in case a, b, c and d are 2 or more, R¹, R², R³ and R⁴ are each in plural being the same or different, a plurality of adjacent R¹s, or a plurality of R²s, or a plurality of R³s, or a plurality of R⁴s may be bonded to each other to form a ring,
2) at least one of A and B is a substituent represented by Formula (A): 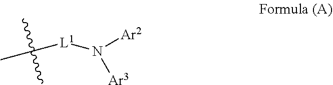
3) a is an integer of 0 to 3; b, c and d are each independently an integer of 0 to 4,
4) Ar¹ is selected from the group consisting of a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;
in Formula (A):
5) L¹ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group;
6) Ar² and Ar³ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or Ar² and Ar³ may be bonded to each other to form a ring,
7) [symbol] represents a position bonded to Formula (1),
8) wherein the aryl group, arylene group, arylamine group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and -L′-N(R^{a})(R^{b}); and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof, and wherein L′ is selected from the group consisting of a single bond; an C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring, and R^{a} and R^{b} are each independently selected from the group consisting of a C₆-C₆₀ aryl group; a fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P.

2. The compound of claim 1, wherein the compound represented by Formula (1) is represented by any one of Formulas (2) to (4): 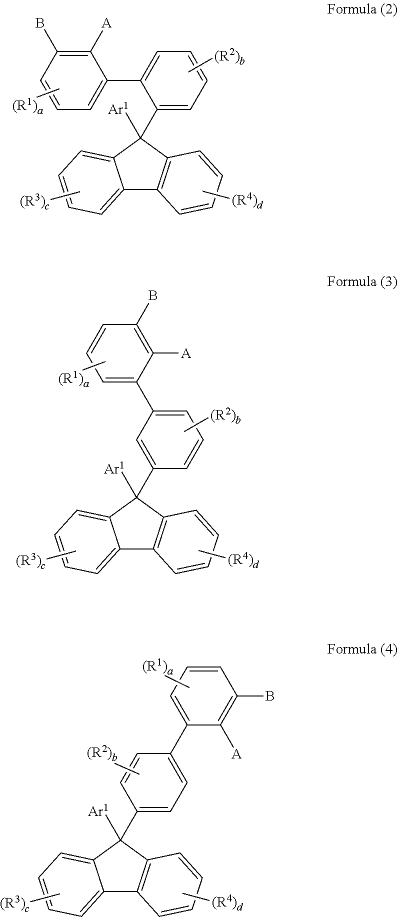
wherein R¹, R², R³, R⁴, A, B, a, b, c, d and Ar¹ are the same as defined in claim 1.

3. The compound of claim 1, wherein the compound represented by Formula (1) is represented by any one of Formulas (5) to (7): 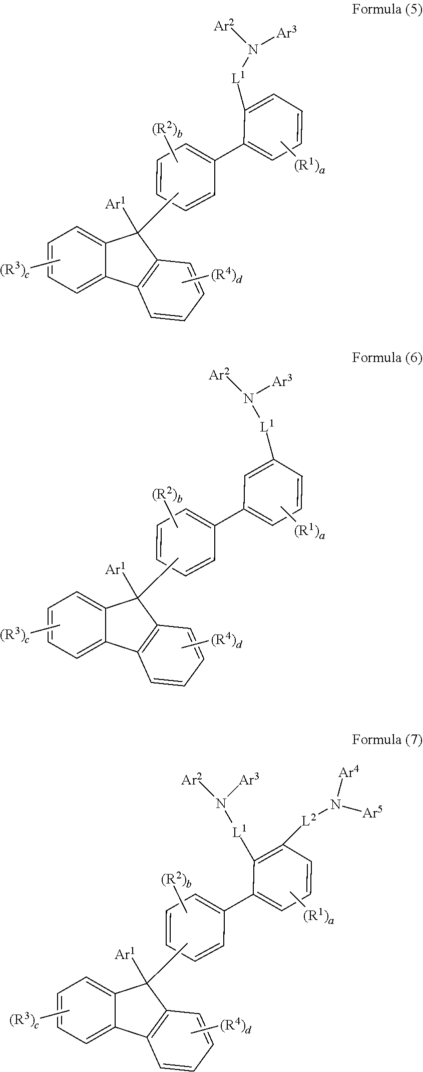
wherein:
1) R¹, R², R³, R⁴, a, b, c, d, L¹, Ar¹, Ar² and Ar³ are the same as defined in claim 1,
2) L² is the same as the definition of L¹, provided that L¹ and L² are the same or different from each other independently,
3) Ar⁴ and Ar⁵ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; or Ar⁴ and Ar⁵ may be bonded to each other to form a ring,
4) a′ is an integer of 0 to 4.

4. The compound of claim 1, wherein the compound represented by Formula (1) is represented by any one of Formulas (8) to (10): 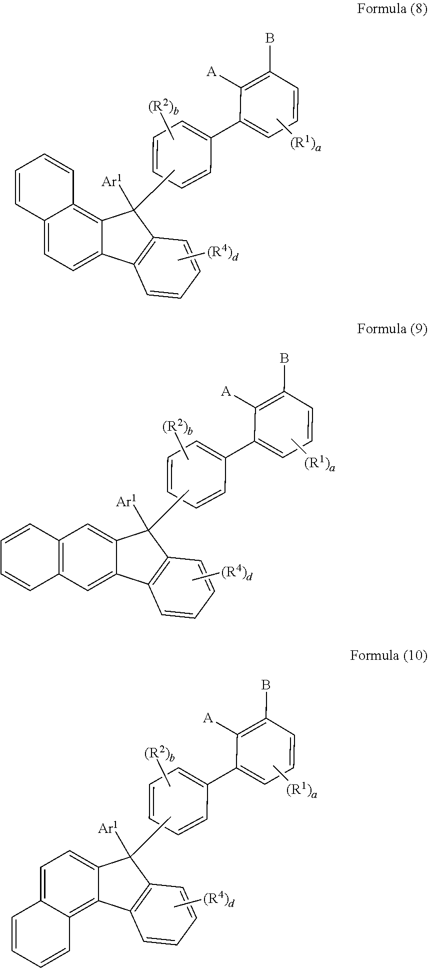
wherein R¹, R², R⁴, A, B, a, b, d and Ar¹ are the same as defined in claim 1.

5. The compound of claim 1, wherein the compound represented by Formula (A) is represented by any one of Formulas (B) to (D): 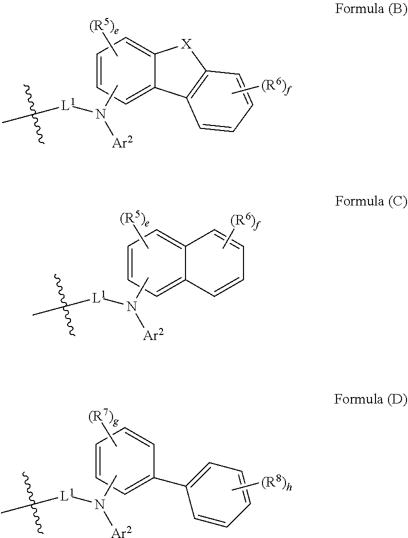
wherein:
1) L¹ and Ar² are the same as defined in claim 1,
2) X is O, S, NR′ or CR′R″,
3) R′ and R″ are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or R′ and R″ may be bonded to each other to form a ring,
4) R⁵, R⁶, R⁷ and R⁸ are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or in case e, f, g and h are 2 or more, R⁵, R⁶, R⁷ and R⁸ are each in plural being the same or different, and a plurality of R⁵ or a plurality of R⁶ or a plurality of R⁷ or a plurality of R⁸ may be bonded to each other to form a ring,
5) e is an integer from 0 to 3, f and g are each independently an integer from 0 to 4, and h is an integer from 0 to 5.

6. The compound of claim 1, wherein the compound represented by Formula (1) is any of following compounds 1-1 to 1-132:

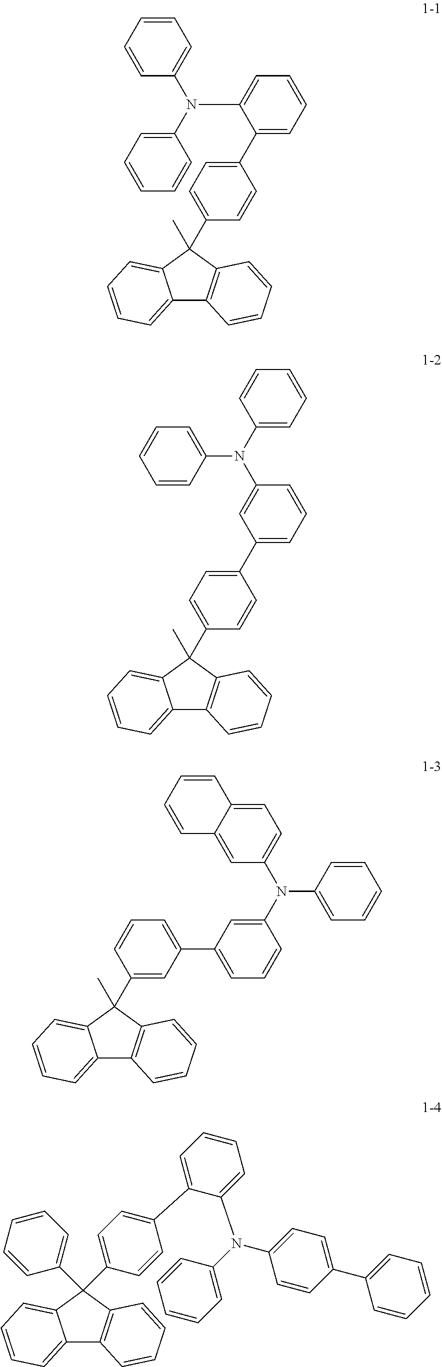

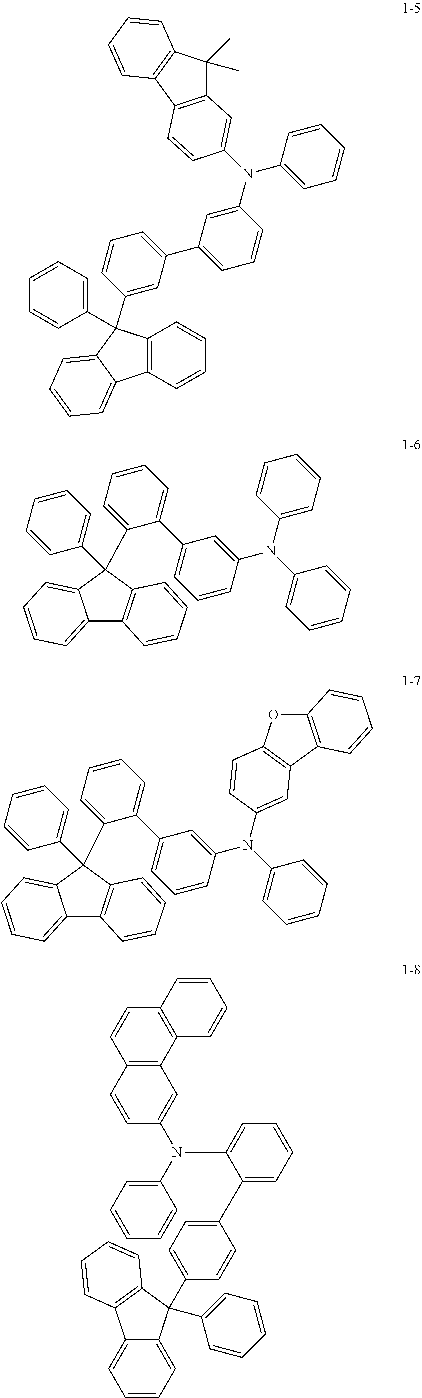 [SMILES: CC1(C)c2ccccc2-c2ccc(N(c3ccccc3)c3cccc(-c4cccc(C5(c6ccccc6)c6ccccc6-c6ccccc65)c4)c3)cc21 ; c1ccc(N(c2ccccc2)c2cccc(-c3ccccc3C3(c4ccccc4)c4ccccc4-c4ccccc43)c2)cc1 ; c1ccc(N(c2cccc(-c3ccccc3C3(c4ccccc4)c4ccccc4-c4ccccc43)c2)c2ccc3oc4ccccc4c3c2)cc1 ; c1ccc(N(c2ccc3ccc4ccccc4c3c2)c2ccccc2-c2ccc(C3(c4ccccc4)c4ccccc4-c4ccccc43)cc2)cc1]

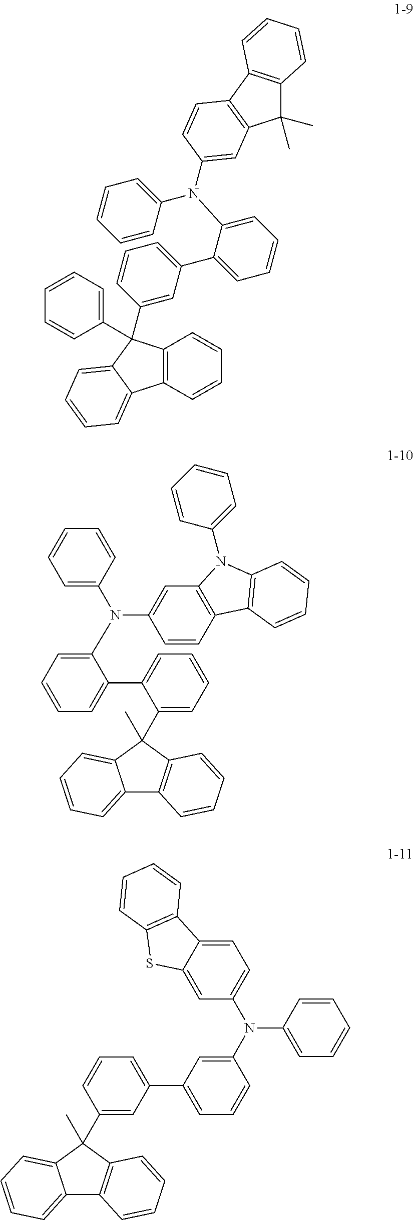 [SMILES: CC1(C)c2ccccc2-c2ccc(N(c3ccccc3)c3ccccc3-c3cccc(C4(c5ccccc5)c5ccccc5-c5ccccc54)c3)cc21 ; CC1(c2ccccc2-c2ccccc2N(c2ccccc2)c2ccc3c4ccccc4n(-c4ccccc4)c3c2)c2ccccc2-c2ccccc21 ; CC1(c2cccc(-c3cccc(N(c4ccccc4)c4ccc5c(c4)sc4ccccc45)c3)c2)c2ccccc2-c2ccccc21]

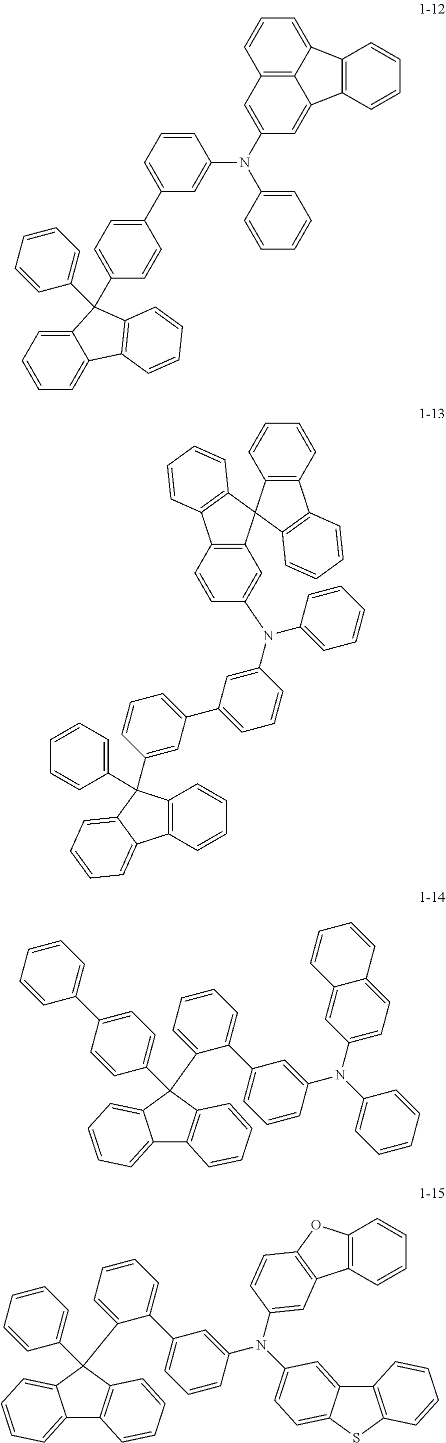 [SMILES: c1ccc(N(c2cccc(-c3ccc(C4(c5ccccc5)c5ccccc5-c5ccccc54)cc3)c2)c2cc3c4c(cccc4c2)-c2ccccc2-3)cc1 ; c1ccc(N(c2cccc(-c3cccc(C4(c5ccccc5)c5ccccc5-c5ccccc54)c3)c2)c2ccc3c(c2)C2(c4ccccc4-c4ccccc42)c2ccccc2-3)cc1 ; c1ccc(-c2ccc(C3(c4ccccc4-c4cccc(N(c5ccccc5)c5ccc6ccccc6c5)c4)c4ccccc4-c4ccccc43)cc2)cc1 ; c1ccc(C2(c3ccccc3-c3cccc(N(c4ccc5oc6ccccc6c5c4)c4ccc5sc6ccccc6c5c4)c3)c3ccccc3-c3ccccc32)cc1]

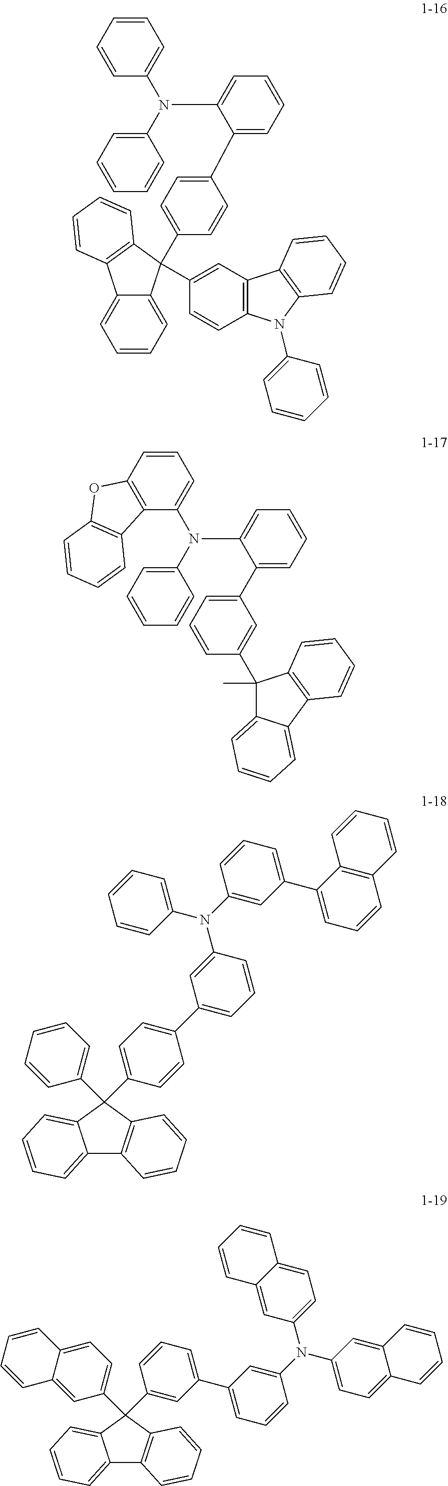 [SMILES: c1ccc(N(c2ccccc2)c2ccccc2-c2ccc(C3(c4ccc5c(c4)c4ccccc4n5-c4ccccc4)c4ccccc4-c4ccccc43)cc2)cc1 ; CC1(c2cccc(-c3ccccc3N(c3ccccc3)c3cccc4oc5ccccc5c34)c2)c2ccccc2-c2ccccc21 ; c1ccc(N(c2cccc(-c3ccc(C4(c5ccccc5)c5ccccc5-c5ccccc54)cc3)c2)c2cccc(-c3cccc4ccccc34)c2)cc1 ; c1cc(-c2cccc(C3(c4ccc5ccccc5c4)c4ccccc4-c4ccccc43)c2)cc(N(c2ccc3ccccc3c2)c2ccc3ccccc3c2)c1]

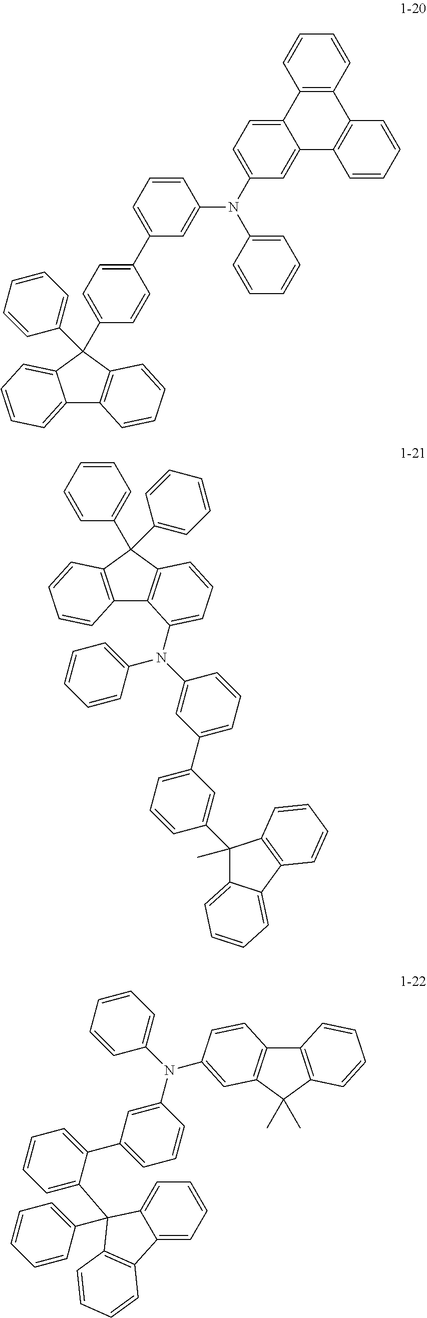

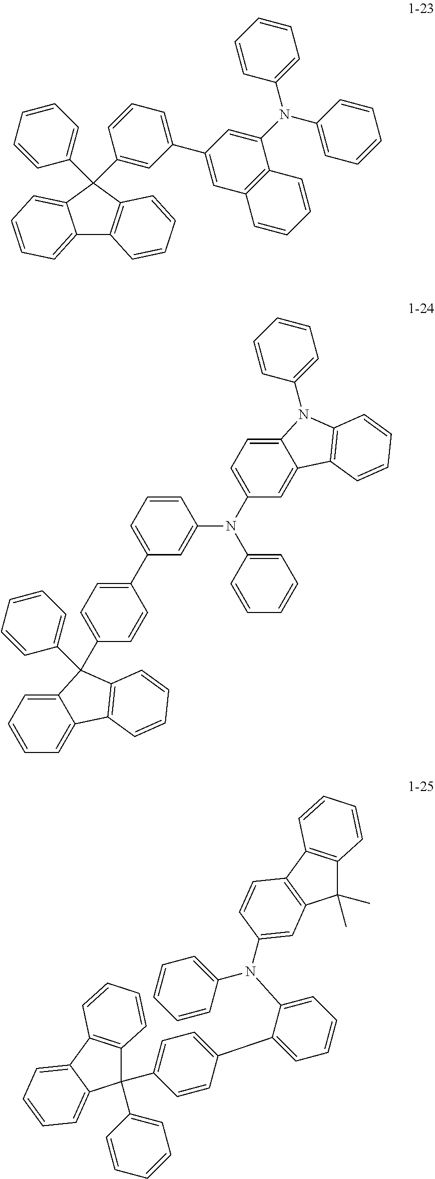 [SMILES: c1ccc(N(c2ccccc2)c2cc(-c3cccc(C4(c5ccccc5)c5ccccc5-c5ccccc54)c3)cc3ccccc23)cc1 ; c1ccc(N(c2cccc(-c3ccc(C4(c5ccccc5)c5ccccc5-c5ccccc54)cc3)c2)c2ccc3c(c2)c2ccccc2n3-c2ccccc2)cc1 ; CC1(C)c2ccccc2-c2ccc(N(c3ccccc3)c3ccccc3-c3ccc(C4(c5ccccc5)c5ccccc5-c5ccccc54)cc3)cc21]

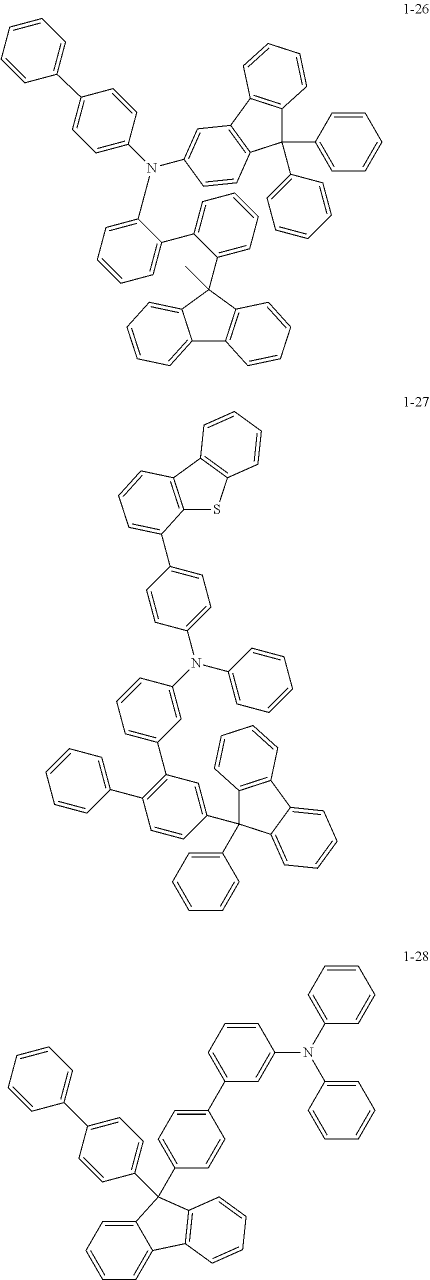 [SMILES: CC1(c2ccccc2-c2ccccc2N(c2ccc(-c3ccccc3)cc2)c2ccc3c(c2)-c2ccccc2C3(c2ccccc2)c2ccccc2)c2ccccc2-c2ccccc21 ; c1ccc(-c2ccc(C3(c4ccccc4)c4ccccc4-c4ccccc43)cc2-c2cccc(N(c3ccccc3)c3ccc(-c4cccc5c4sc4ccccc45)cc3)c2)cc1 ; c1ccc(-c2ccc(C3(c4ccc(-c5cccc(N(c6ccccc6)c6ccccc6)c5)cc4)c4ccccc4-c4ccccc43)cc2)cc1]

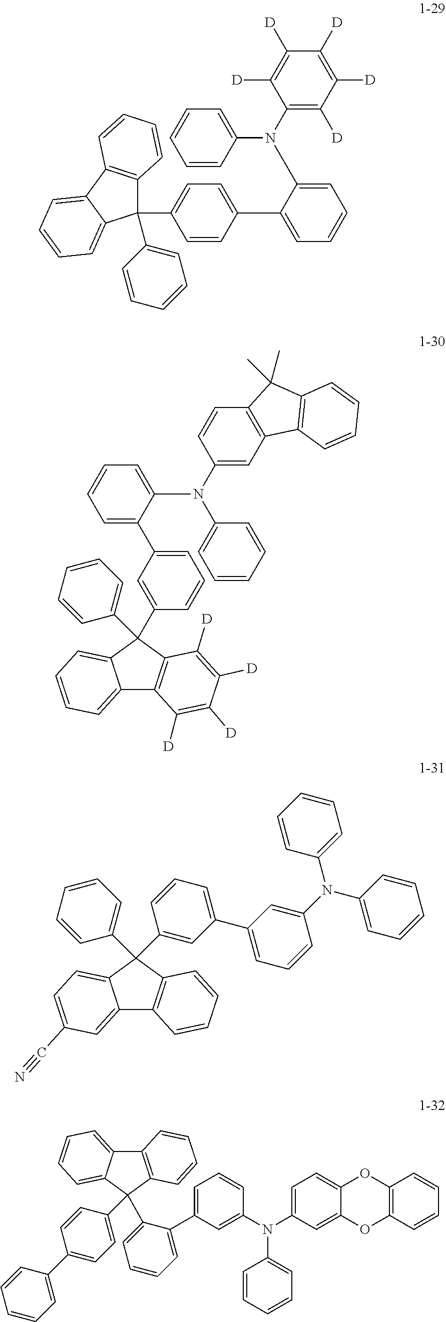 [SMILES: [2H]c1c([2H])c([2H])c(N(c2ccccc2)c2ccccc2-c2ccc(C3(c4ccccc4)c4ccccc4-c4ccccc43)cc2)c([2H])c1[2H] ; [2H]c1c([2H])c([2H])c2c(c1[2H])-c1ccccc1C2(c1ccccc1)c1cccc(-c2ccccc2N(c2ccccc2)c2ccc3c(c2)-c2ccccc2C3(C)C)c1 ; N#Cc1ccc2c(c1)-c1ccccc1C2(c1ccccc1)c1cccc(-c2cccc(N(c3ccccc3)c3ccccc3)c2)c1 ; c1ccc(-c2ccc(C3(c4ccccc4-c4cccc(N(c5ccccc5)c5ccc6c(c5)Oc5ccccc5O6)c4)c4ccccc4-c4ccccc43)cc2)cc1]

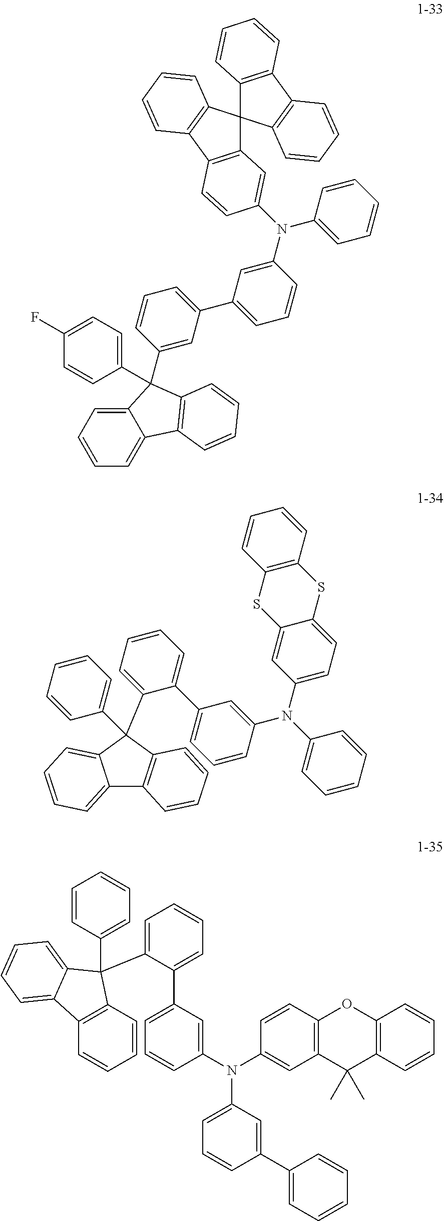 [SMILES: Fc1ccc(C2(c3cccc(-c4cccc(N(c5ccccc5)c5ccc6c(c5)C5(c7ccccc7-c7ccccc75)c5ccccc5-6)c4)c3)c3ccccc3-c3ccccc32)cc1 ; c1ccc(N(c2cccc(-c3ccccc3C3(c4ccccc4)c4ccccc4-c4ccccc43)c2)c2ccc3c(c2)Sc2ccccc2S3)cc1 ; CC1(C)c2ccccc2Oc2ccc(N(c3cccc(-c4ccccc4)c3)c3cccc(-c4ccccc4C4(c5ccccc5)c5ccccc5-c5ccccc54)c3)cc21]

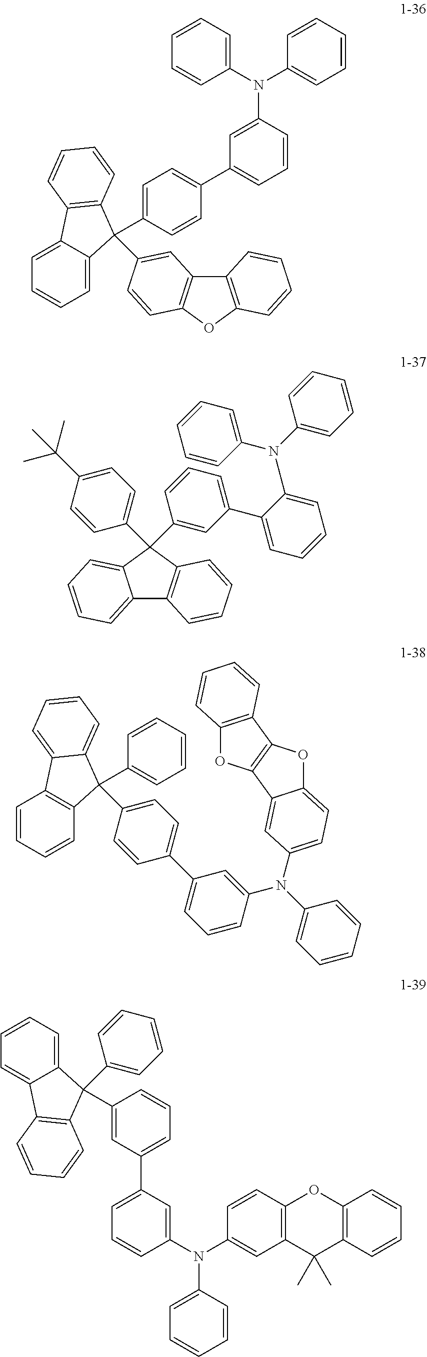 [SMILES: c1ccc(N(c2ccccc2)c2cccc(-c3ccc(C4(c5ccc6oc7ccccc7c6c5)c5ccccc5-c5ccccc54)cc3)c2)cc1 ; CC(C)(C)c1ccc(C2(c3cccc(-c4ccccc4N(c4ccccc4)c4ccccc4)c3)c3ccccc3-c3ccccc32)cc1 ; c1ccc(N(c2cccc(-c3ccc(C4(c5ccccc5)c5ccccc5-c5ccccc54)cc3)c2)c2ccc3oc4c5ccccc5oc4c3c2)cc1 ; CC1(C)c2ccccc2Oc2ccc(N(c3ccccc3)c3cccc(-c4cccc(C5(c6ccccc6)c6ccccc6-c6ccccc65)c4)c3)cc21]

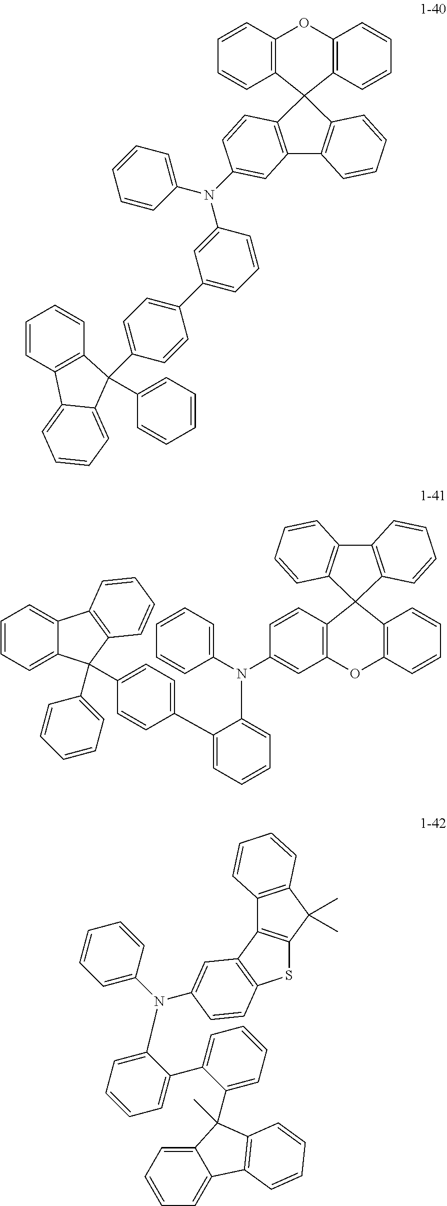 [SMILES: c1ccc(N(c2cccc(-c3ccc(C4(c5ccccc5)c5ccccc5-c5ccccc54)cc3)c2)c2ccc3c(c2)-c2ccccc2C32c3ccccc3Oc3ccccc32)cc1 ; c1ccc(N(c2ccc3c(c2)Oc2ccccc2C32c3ccccc3-c3ccccc32)c2ccccc2-c2ccc(C3(c4ccccc4)c4ccccc4-c4ccccc43)cc2)cc1 ; CC1(C)c2ccccc2-c2c1sc1ccc(N(c3ccccc3)c3ccccc3-c3ccccc3C3(C)c4ccccc4-c4ccccc43)cc21]

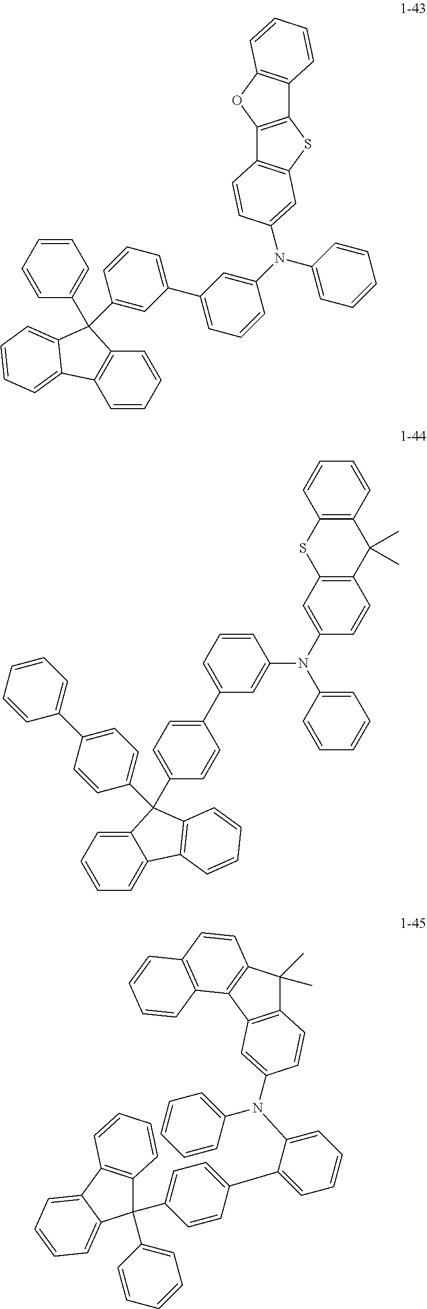 [SMILES: c1ccc(N(c2cccc(-c3cccc(C4(c5ccccc5)c5ccccc5-c5ccccc54)c3)c2)c2ccc3c(c2)sc2c4ccccc4oc32)cc1 ; CC1(C)c2ccccc2Sc2cc(N(c3ccccc3)c3cccc(-c4ccc(C5(c6ccc(-c7ccccc7)cc6)c6ccccc6-c6ccccc65)cc4)c3)ccc21 ; CC1(C)c2ccc(N(c3ccccc3)c3ccccc3-c3ccc(C4(c5ccccc5)c5ccccc5-c5ccccc54)cc3)cc2-c2c1ccc1ccccc21]

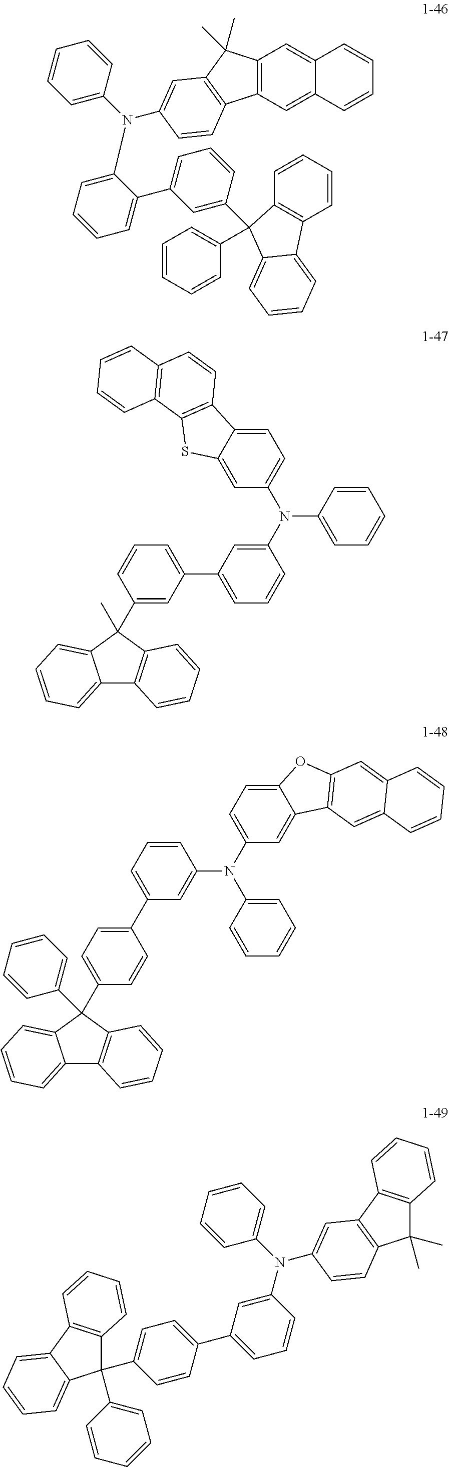 [SMILES: CC1(C)c2cc(N(c3ccccc3)c3ccccc3-c3cccc(C4(c5ccccc5)c5ccccc5-c5ccccc54)c3)ccc2-c2cc3ccccc3cc21 ; CC1(c2cccc(-c3cccc(N(c4ccccc4)c4ccc5c(c4)sc4c6ccccc6ccc54)c3)c2)c2ccccc2-c2ccccc21 ; c1ccc(N(c2cccc(-c3ccc(C4(c5ccccc5)c5ccccc5-c5ccccc54)cc3)c2)c2ccc3oc4cc5ccccc5cc4c3c2)cc1 ; CC1(C)c2ccccc2-c2cc(N(c3ccccc3)c3cccc(-c4ccc(C5(c6ccccc6)c6ccccc6-c6ccccc65)cc4)c3)ccc21]

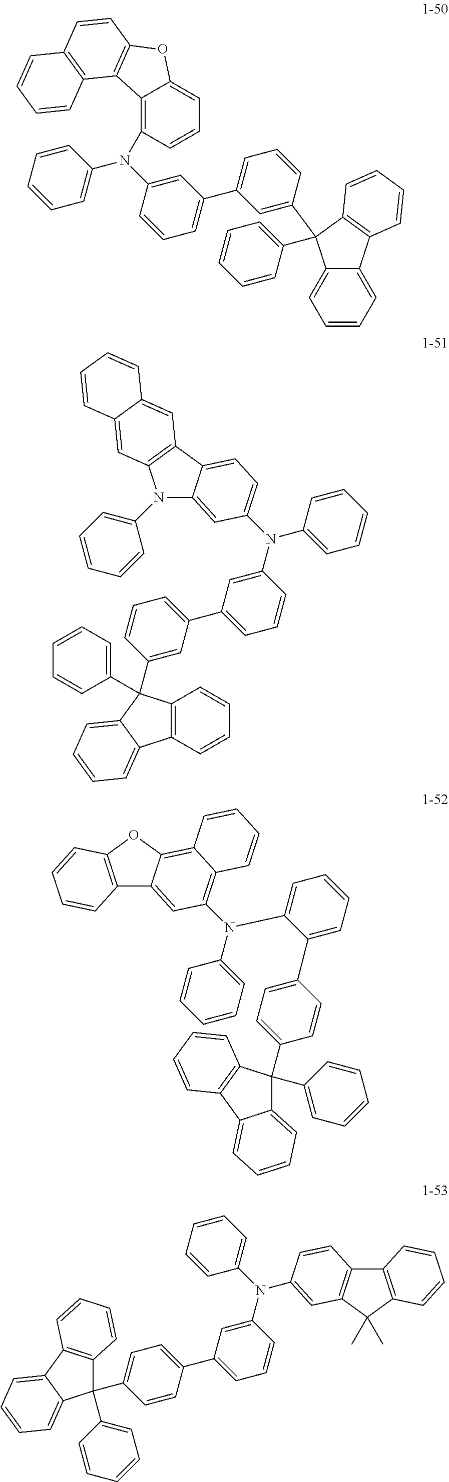 [SMILES: c1ccc(N(c2cccc(-c3cccc(C4(c5ccccc5)c5ccccc5-c5ccccc54)c3)c2)c2cccc3oc4ccc5ccccc5c4c23)cc1 ; c1ccc(N(c2cccc(-c3cccc(C4(c5ccccc5)c5ccccc5-c5ccccc54)c3)c2)c2ccc3c4cc5ccccc5cc4n(-c4ccccc4)c3c2)cc1 ; c1ccc(N(c2ccccc2-c2ccc(C3(c4ccccc4)c4ccccc4-c4ccccc43)cc2)c2cc3c4ccccc4oc3c3ccccc23)cc1 ; CC1(C)c2ccccc2-c2ccc(N(c3ccccc3)c3cccc(-c4ccc(C5(c6ccccc6)c6ccccc6-c6ccccc65)cc4)c3)cc21]

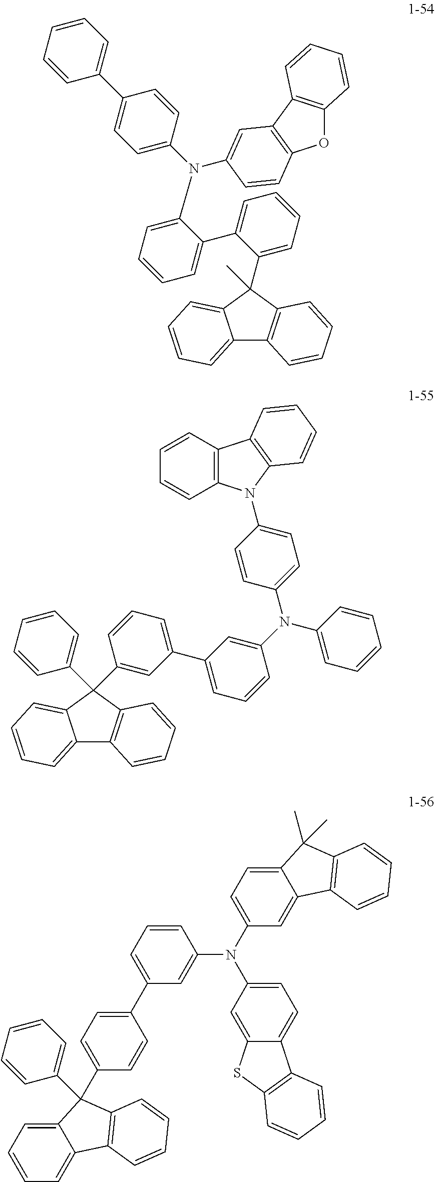

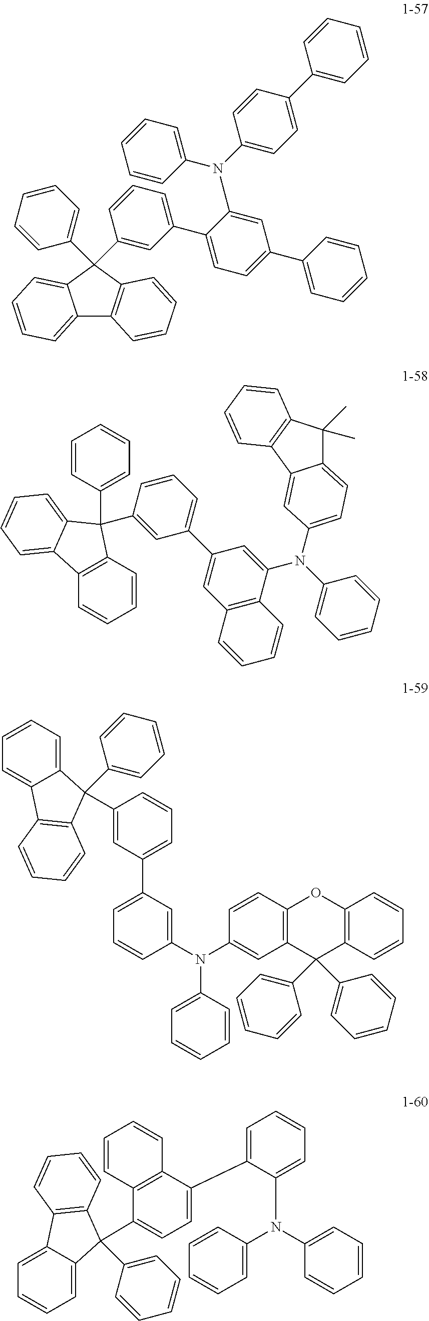 [SMILES: c1ccc(-c2ccc(N(c3ccccc3)c3cc(-c4ccccc4)ccc3-c3cccc(C4(c5ccccc5)c5ccccc5-c5ccccc54)c3)cc2)cc1 ; CC1(C)c2ccccc2-c2cc(N(c3ccccc3)c3cc(-c4cccc(C5(c6ccccc6)c6ccccc6-c6ccccc65)c4)cc4ccccc34)ccc21 ; c1ccc(N(c2cccc(-c3cccc(C4(c5ccccc5)c5ccccc5-c5ccccc54)c3)c2)c2ccc3c(c2)C(c2ccccc2)(c2ccccc2)c2ccccc2O3)cc1 ; c1ccc(N(c2ccccc2)c2ccccc2-c2ccc(C3(c4ccccc4)c4ccccc4-c4ccccc43)c3ccccc23)cc1]

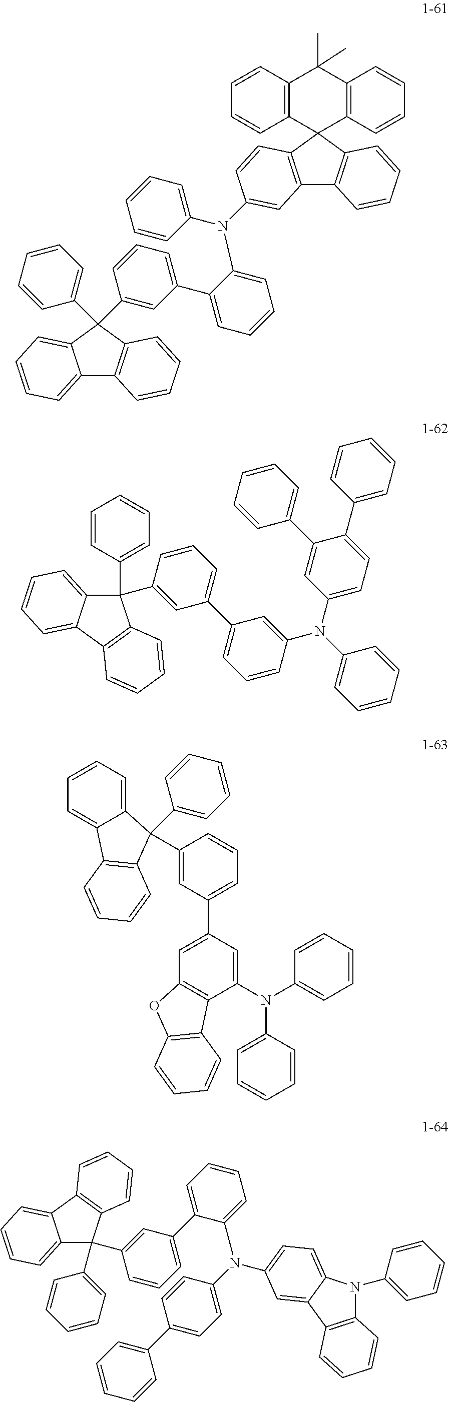 [SMILES: CC1(C)c2ccccc2C2(c3ccccc3-c3cc(N(c4ccccc4)c4ccccc4-c4cccc(C5(c6ccccc6)c6ccccc6-c6ccccc65)c4)ccc32)c2ccccc21 ; c1ccc(-c2ccc(N(c3ccccc3)c3cccc(-c4cccc(C5(c6ccccc6)c6ccccc6-c6ccccc65)c4)c3)cc2-c2ccccc2)cc1 ; c1ccc(N(c2ccccc2)c2cc(-c3cccc(C4(c5ccccc5)c5ccccc5-c5ccccc54)c3)cc3oc4ccccc4c23)cc1 ; c1ccc(-c2ccc(N(c3ccc4c(c3)c3ccccc3n4-c3ccccc3)c3ccccc3-c3cccc(C4(c5ccccc5)c5ccccc5-c5ccccc54)c3)cc2)cc1]

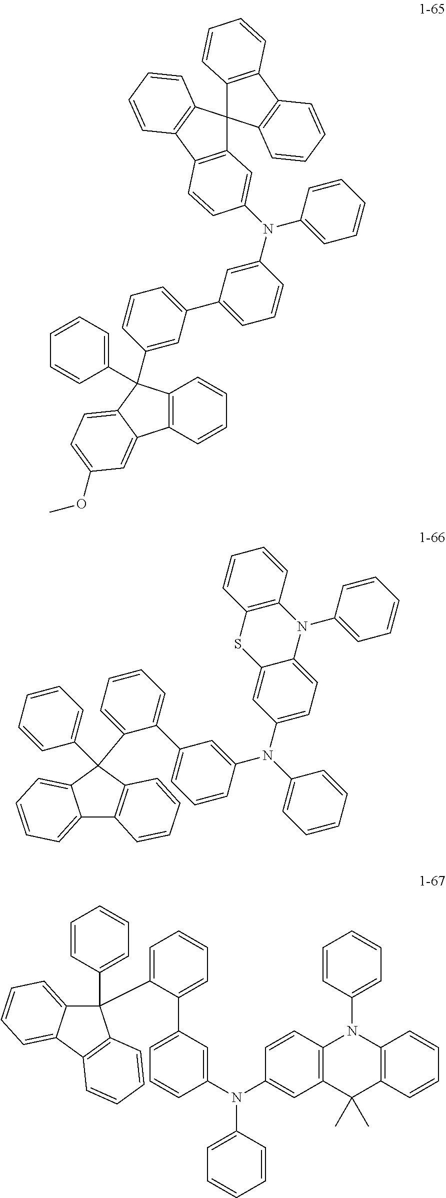 [SMILES: COc1ccc2c(c1)-c1ccccc1C2(c1ccccc1)c1cccc(-c2cccc(N(c3ccccc3)c3ccc4c(c3)C3(c5ccccc5-c5ccccc53)c3ccccc3-4)c2)c1 ; c1ccc(N(c2cccc(-c3ccccc3C3(c4ccccc4)c4ccccc4-c4ccccc43)c2)c2ccc3c(c2)Sc2ccccc2N3c2ccccc2)cc1 ; CC1(C)c2ccccc2N(c2ccccc2)c2ccc(N(c3ccccc3)c3cccc(-c4ccccc4C4(c5ccccc5)c5ccccc5-c5ccccc54)c3)cc21]

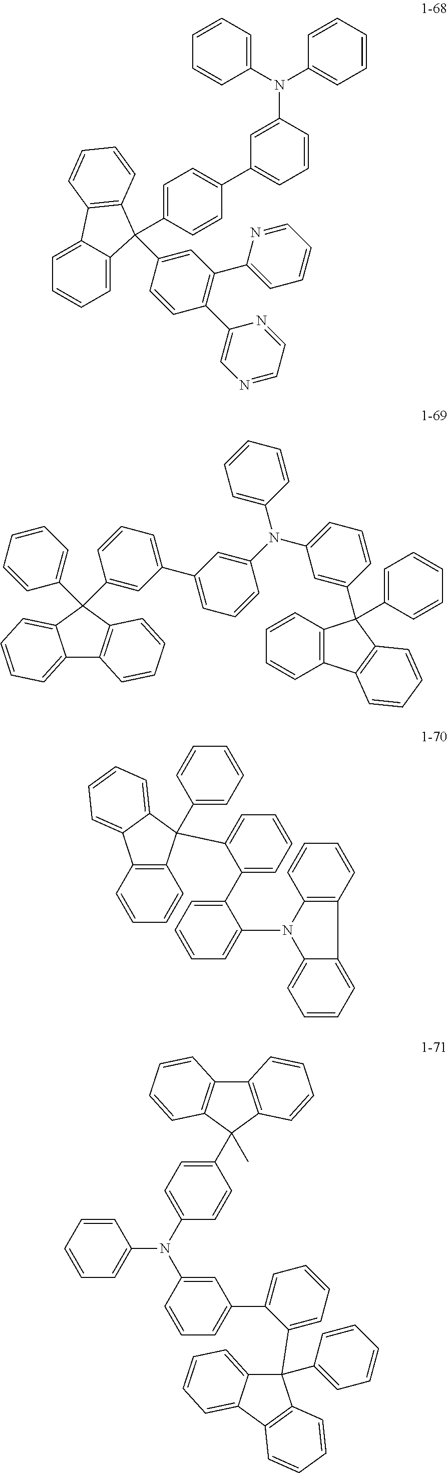 [SMILES: c1ccc(N(c2ccccc2)c2cccc(-c3ccc(C4(c5ccc(-c6cnccn6)c(-c6ccccn6)c5)c5ccccc5-c5ccccc54)cc3)c2)cc1 ; c1ccc(N(c2cccc(-c3cccc(C4(c5ccccc5)c5ccccc5-c5ccccc54)c3)c2)c2cccc(C3(c4ccccc4)c4ccccc4-c4ccccc43)c2)cc1 ; c1ccc(C2(c3ccccc3-c3ccccc3-n3c4ccccc4c4ccccc43)c3ccccc3-c3ccccc32)cc1 ; CC1(c2ccc(N(c3ccccc3)c3cccc(-c4ccccc4C4(c5ccccc5)c5ccccc5-c5ccccc54)c3)cc2)c2ccccc2-c2ccccc21]

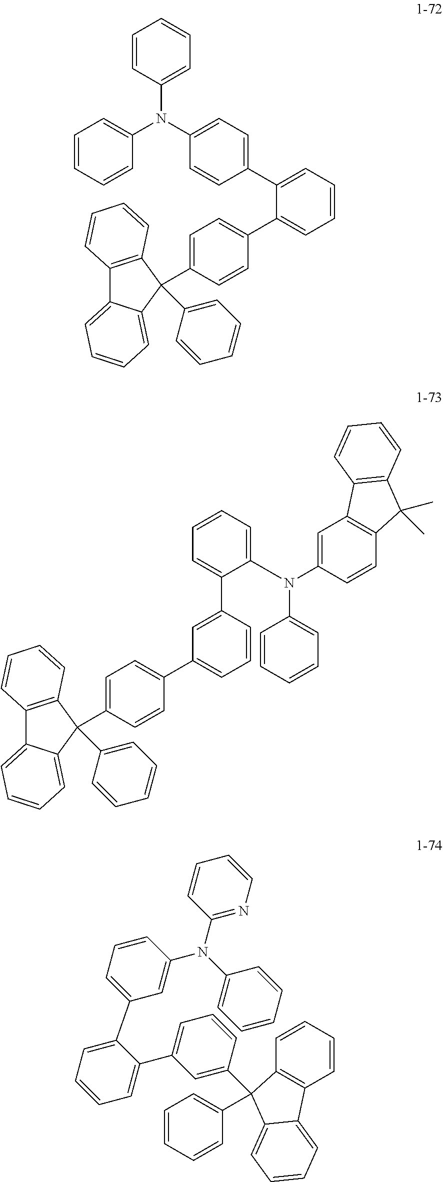 [SMILES: c1ccc(N(c2ccccc2)c2ccc(-c3ccccc3-c3ccc(C4(c5ccccc5)c5ccccc5-c5ccccc54)cc3)cc2)cc1 ; CC1(C)c2ccccc2-c2cc(N(c3ccccc3)c3ccccc3-c3cccc(-c4ccc(C5(c6ccccc6)c6ccccc6-c6ccccc65)cc4)c3)ccc21 ; c1ccc(N(c2cccc(-c3ccccc3-c3cccc(C4(c5ccccc5)c5ccccc5-c5ccccc54)c3)c2)c2ccccn2)cc1]

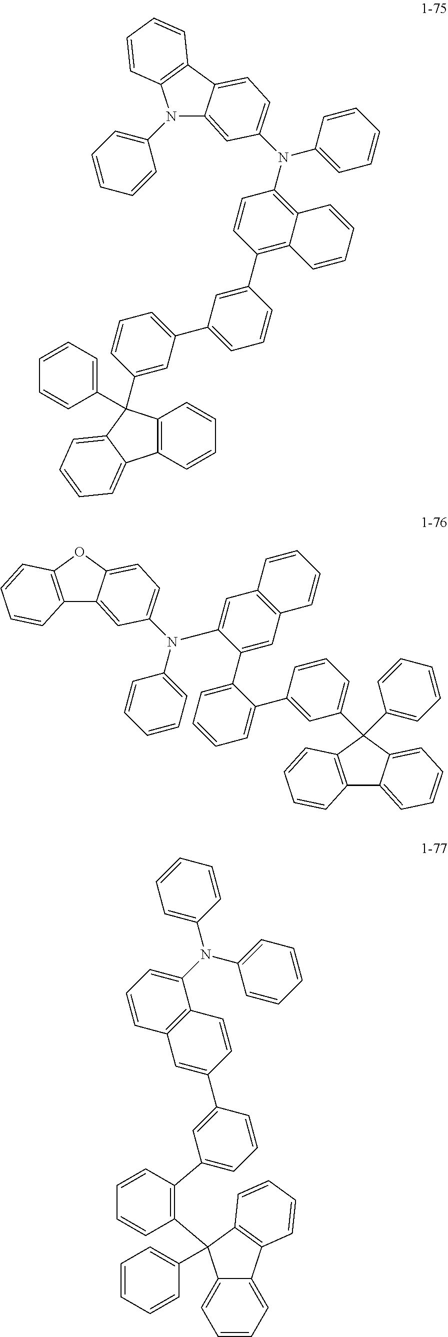 [SMILES: c1ccc(N(c2ccc3c4ccccc4n(-c4ccccc4)c3c2)c2ccc(-c3cccc(-c4cccc(C5(c6ccccc6)c6ccccc6-c6ccccc65)c4)c3)c3ccccc23)cc1 ; c1ccc(N(c2ccc3oc4ccccc4c3c2)c2cc3ccccc3cc2-c2ccccc2-c2cccc(C3(c4ccccc4)c4ccccc4-c4ccccc43)c2)cc1 ; c1ccc(N(c2ccccc2)c2cccc3cc(-c4cccc(-c5ccccc5C5(c6ccccc6)c6ccccc6-c6ccccc65)c4)ccc23)cc1]

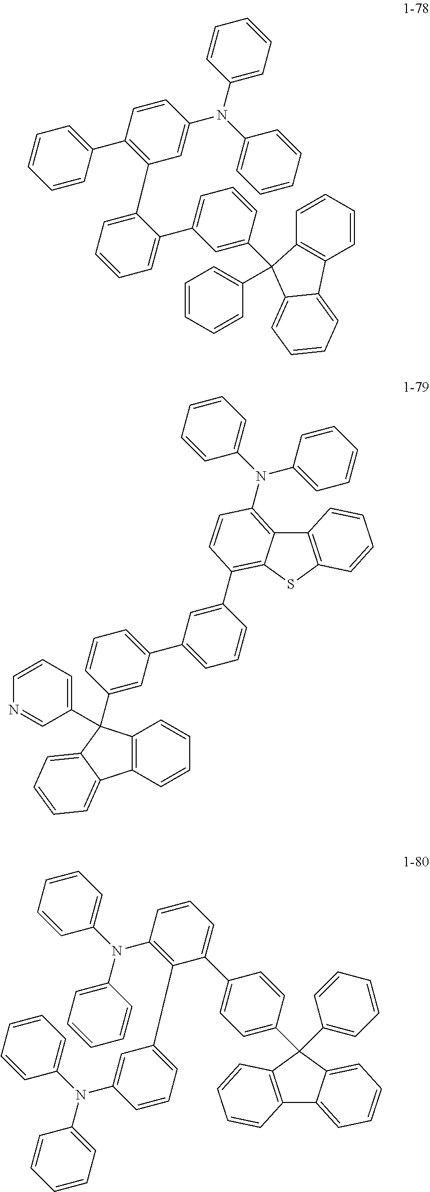 [SMILES: c1ccc(-c2ccc(N(c3ccccc3)c3ccccc3)cc2-c2ccccc2-c2cccc(C3(c4ccccc4)c4ccccc4-c4ccccc43)c2)cc1 ; c1ccc(N(c2ccccc2)c2ccc(-c3cccc(-c4cccc(C5(c6cccnc6)c6ccccc6-c6ccccc65)c4)c3)c3sc4ccccc4c23)cc1 ; c1ccc(N(c2ccccc2)c2cccc(-c3c(-c4ccc(C5(c6ccccc6)c6ccccc6-c6ccccc65)cc4)cccc3N(c3ccccc3)c3ccccc3)c2)cc1]

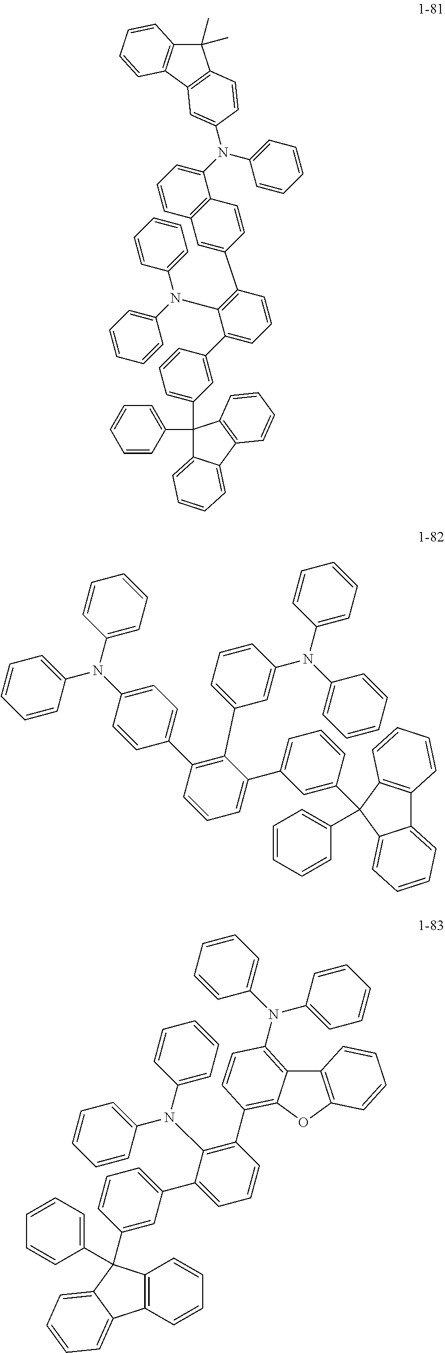 [SMILES: CC1(C)c2ccccc2-c2cc(N(c3ccccc3)c3cccc4cc(-c5cccc(-c6cccc(C7(c8ccccc8)c8ccccc8-c8ccccc87)c6)c5N(c5ccccc5)c5ccccc5)ccc34)ccc21 ; c1ccc(N(c2ccccc2)c2ccc(-c3cccc(-c4cccc(C5(c6ccccc6)c6ccccc6-c6ccccc65)c4)c3-c3cccc(N(c4ccccc4)c4ccccc4)c3)cc2)cc1 ; c1ccc(N(c2ccccc2)c2c(-c3cccc(C4(c5ccccc5)c5ccccc5-c5ccccc54)c3)cccc2-c2ccc(N(c3ccccc3)c3ccccc3)c3c2oc2ccccc23)cc1]

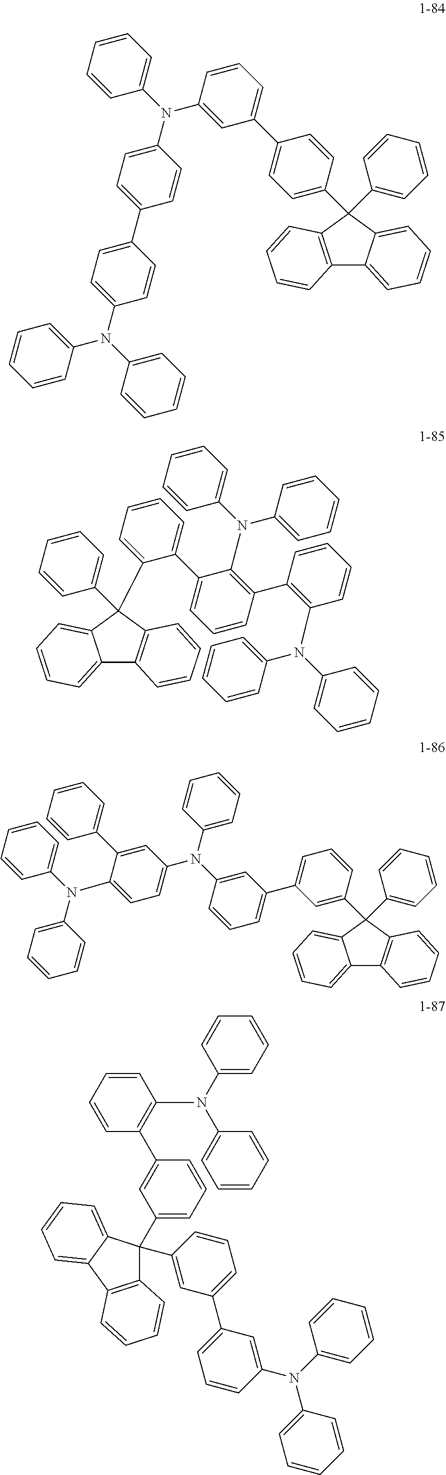

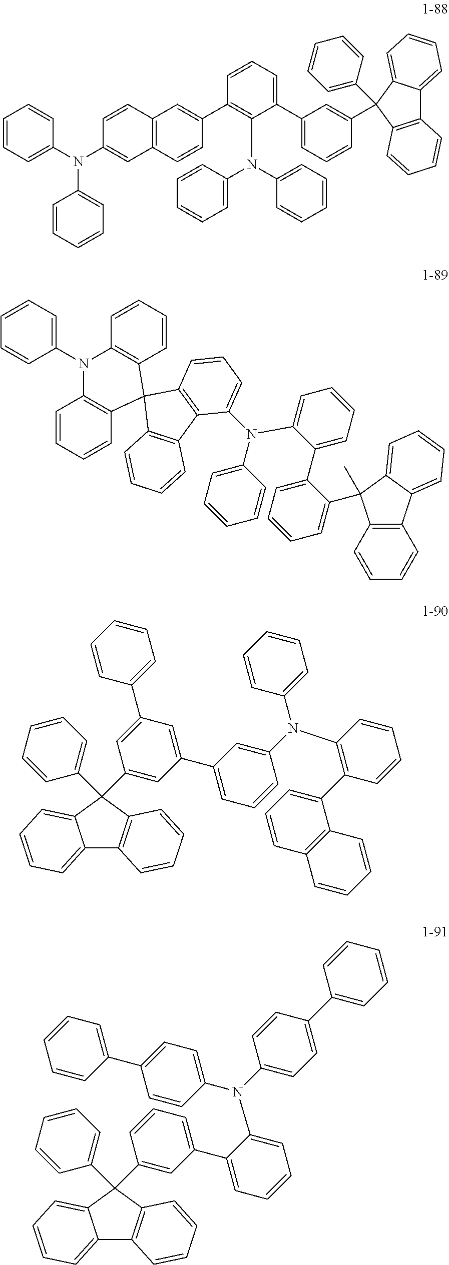 [SMILES: c1ccc(N(c2ccccc2)c2ccc3cc(-c4cccc(-c5cccc(C6(c7ccccc7)c7ccccc7-c7ccccc76)c5)c4N(c4ccccc4)c4ccccc4)ccc3c2)cc1 ; CC1(c2ccccc2-c2ccccc2N(c2ccccc2)c2cccc3c2-c2ccccc2C32c3ccccc3N(c3ccccc3)c3ccccc32)c2ccccc2-c2ccccc21 ; c1ccc(-c2cc(-c3cccc(N(c4ccccc4)c4ccccc4-c4cccc5ccccc45)c3)cc(C3(c4ccccc4)c4ccccc4-c4ccccc43)c2)cc1 ; c1ccc(-c2ccc(N(c3ccc(-c4ccccc4)cc3)c3ccccc3-c3cccc(C4(c5ccccc5)c5ccccc5-c5ccccc54)c3)cc2)cc1]

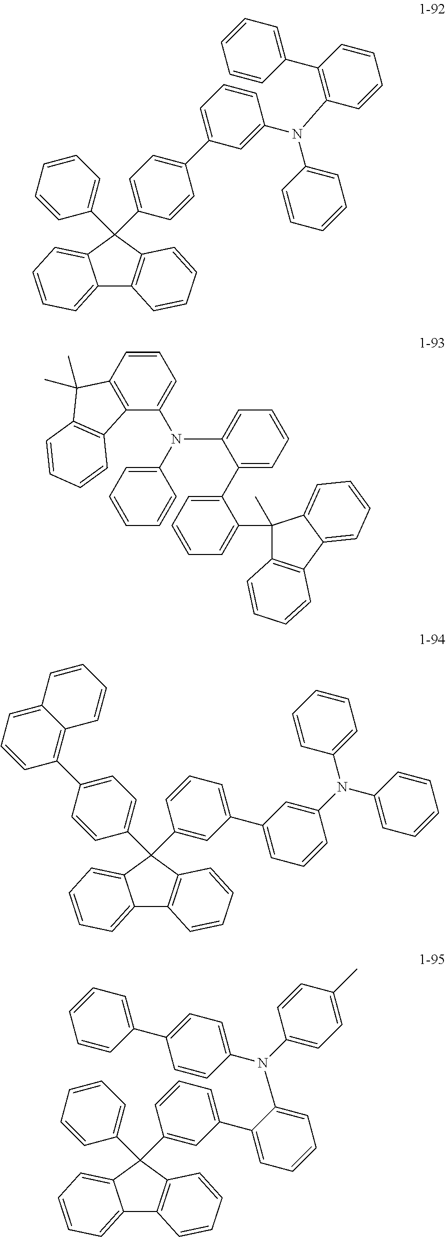

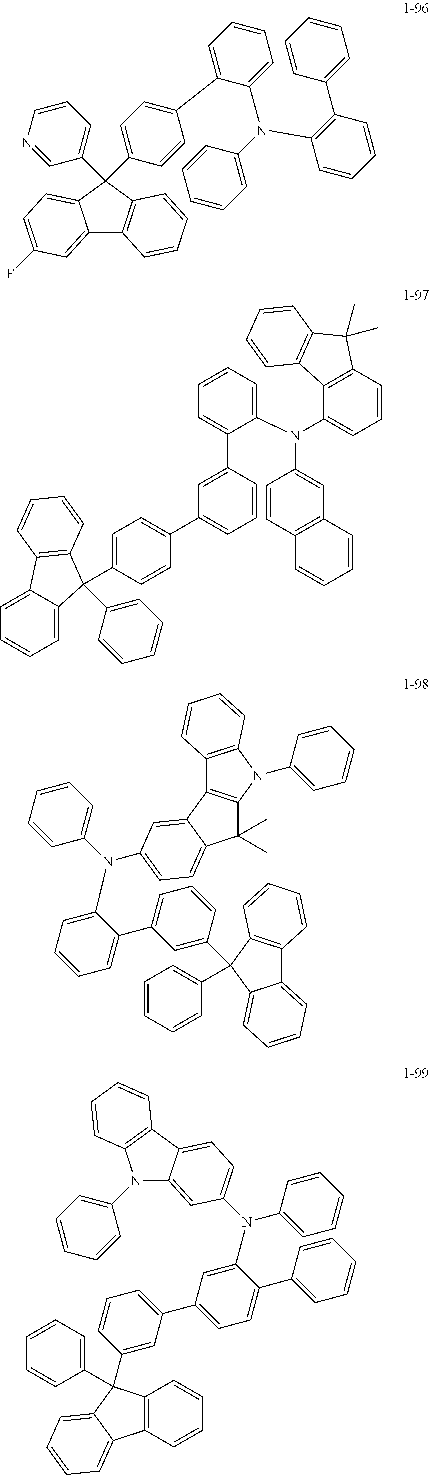 [SMILES: Fc1ccc2c(c1)-c1ccccc1C2(c1ccc(-c2ccccc2N(c2ccccc2)c2ccccc2-c2ccccc2)cc1)c1cccnc1 ; CC1(C)c2ccccc2-c2c(N(c3ccc4ccccc4c3)c3ccccc3-c3cccc(-c4ccc(C5(c6ccccc6)c6ccccc6-c6ccccc65)cc4)c3)cccc21 ; CC1(C)c2ccc(N(c3ccccc3)c3ccccc3-c3cccc(C4(c5ccccc5)c5ccccc5-c5ccccc54)c3)cc2-c2c1n(-c1ccccc1)c1ccccc21 ; c1ccc(-c2ccc(-c3cccc(C4(c5ccccc5)c5ccccc5-c5ccccc54)c3)cc2N(c2ccccc2)c2ccc3c4ccccc4n(-c4ccccc4)c3c2)cc1]

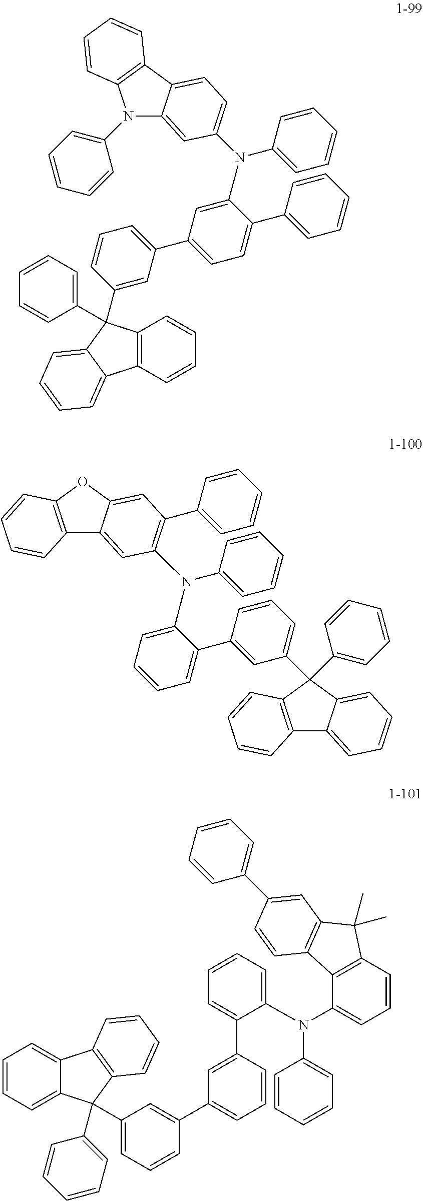 [SMILES: c1ccc(-c2ccc(-c3cccc(C4(c5ccccc5)c5ccccc5-c5ccccc54)c3)cc2N(c2ccccc2)c2ccc3c4ccccc4n(-c4ccccc4)c3c2)cc1 ; c1ccc(-c2cc3oc4ccccc4c3cc2N(c2ccccc2)c2ccccc2-c2cccc(C3(c4ccccc4)c4ccccc4-c4ccccc43)c2)cc1 ; CC1(C)c2cc(-c3ccccc3)ccc2-c2c(N(c3ccccc3)c3ccccc3-c3cccc(-c4cccc(C5(c6ccccc6)c6ccccc6-c6ccccc65)c4)c3)cccc21]

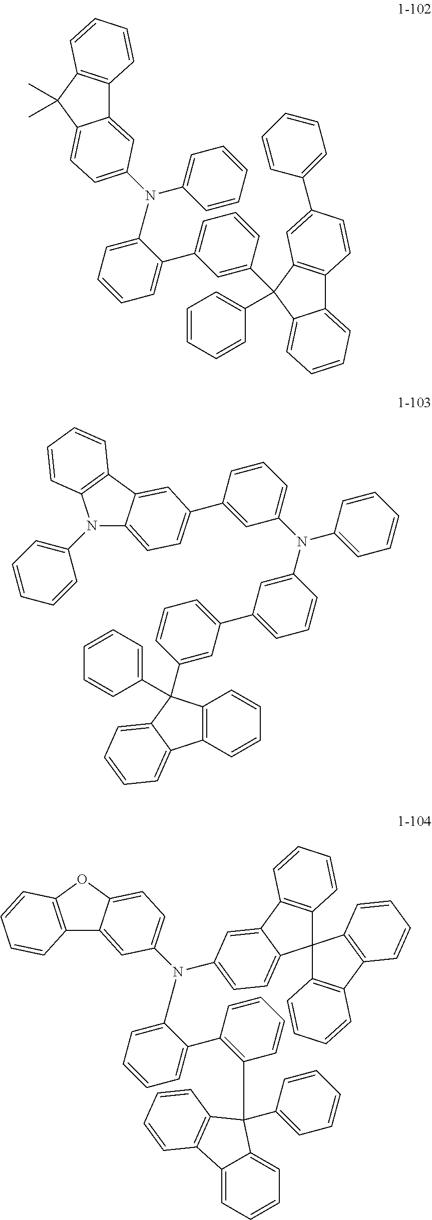 [SMILES: CC1(C)c2ccccc2-c2cc(N(c3ccccc3)c3ccccc3-c3cccc(C4(c5ccccc5)c5ccccc5-c5ccc(-c6ccccc6)cc54)c3)ccc21 ; c1ccc(N(c2cccc(-c3cccc(C4(c5ccccc5)c5ccccc5-c5ccccc54)c3)c2)c2cccc(-c3ccc4c(c3)c3ccccc3n4-c3ccccc3)c2)cc1 ; c1ccc(C2(c3ccccc3-c3ccccc3N(c3ccc4c(c3)-c3ccccc3C43c4ccccc4-c4ccccc43)c3ccc4oc5ccccc5c4c3)c3ccccc3-c3ccccc32)cc1]

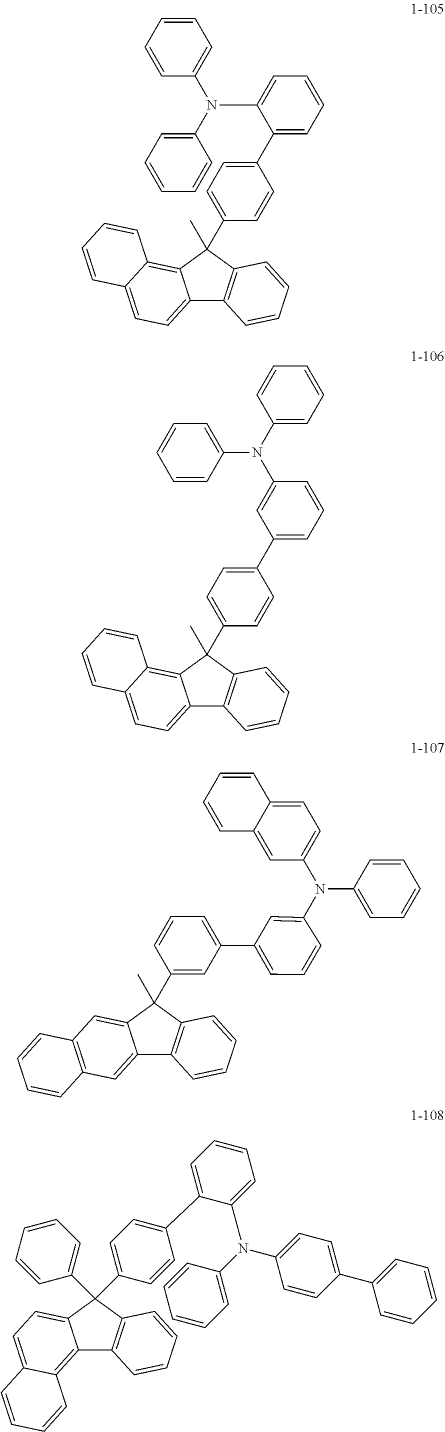

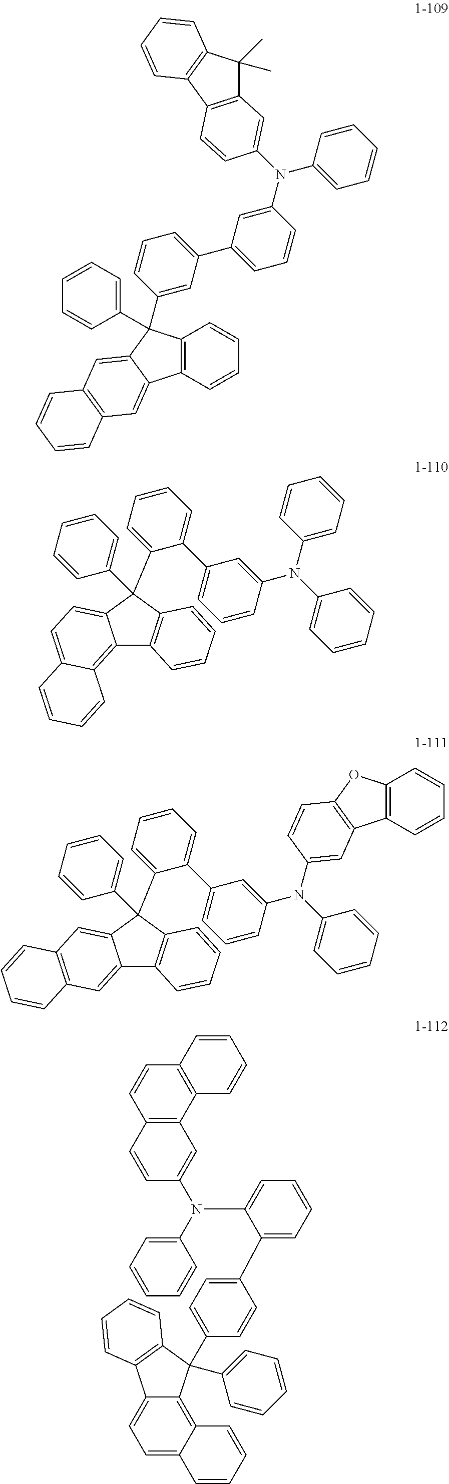 [SMILES: CC1(C)c2ccccc2-c2ccc(N(c3ccccc3)c3cccc(-c4cccc(C5(c6ccccc6)c6ccccc6-c6cc7ccccc7cc65)c4)c3)cc21 ; c1ccc(N(c2ccccc2)c2cccc(-c3ccccc3C3(c4ccccc4)c4ccccc4-c4c3ccc3ccccc43)c2)cc1 ; c1ccc(N(c2cccc(-c3ccccc3C3(c4ccccc4)c4ccccc4-c4cc5ccccc5cc43)c2)c2ccc3oc4ccccc4c3c2)cc1 ; c1ccc(N(c2ccc3ccc4ccccc4c3c2)c2ccccc2-c2ccc(C3(c4ccccc4)c4ccccc4-c4ccc5ccccc5c43)cc2)cc1]

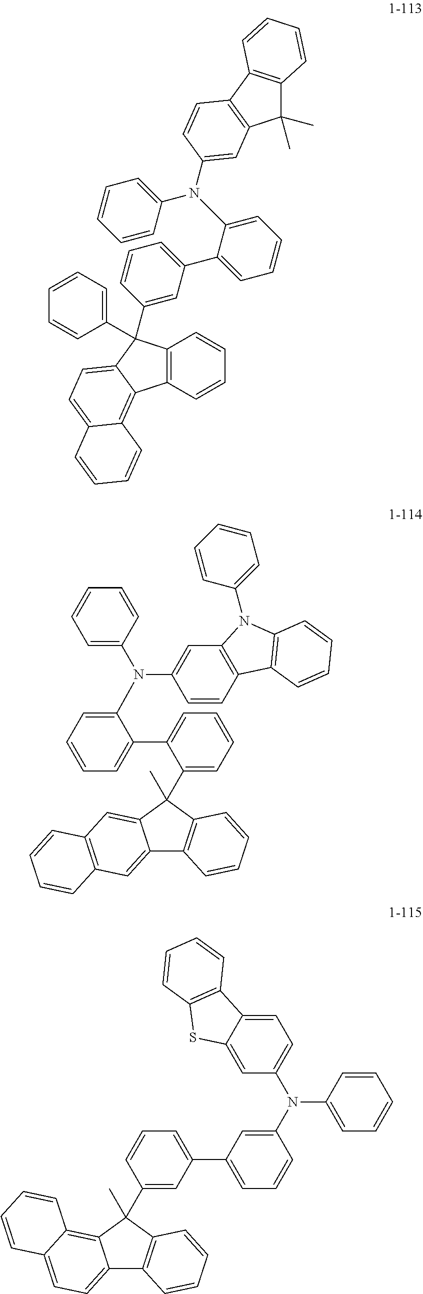 [SMILES: CC1(C)c2ccccc2-c2ccc(N(c3ccccc3)c3ccccc3-c3cccc(C4(c5ccccc5)c5ccccc5-c5c4ccc4ccccc54)c3)cc21 ; CC1(c2ccccc2-c2ccccc2N(c2ccccc2)c2ccc3c4ccccc4n(-c4ccccc4)c3c2)c2ccccc2-c2cc3ccccc3cc21 ; CC1(c2cccc(-c3cccc(N(c4ccccc4)c4ccc5c(c4)sc4ccccc45)c3)c2)c2ccccc2-c2ccc3ccccc3c21]

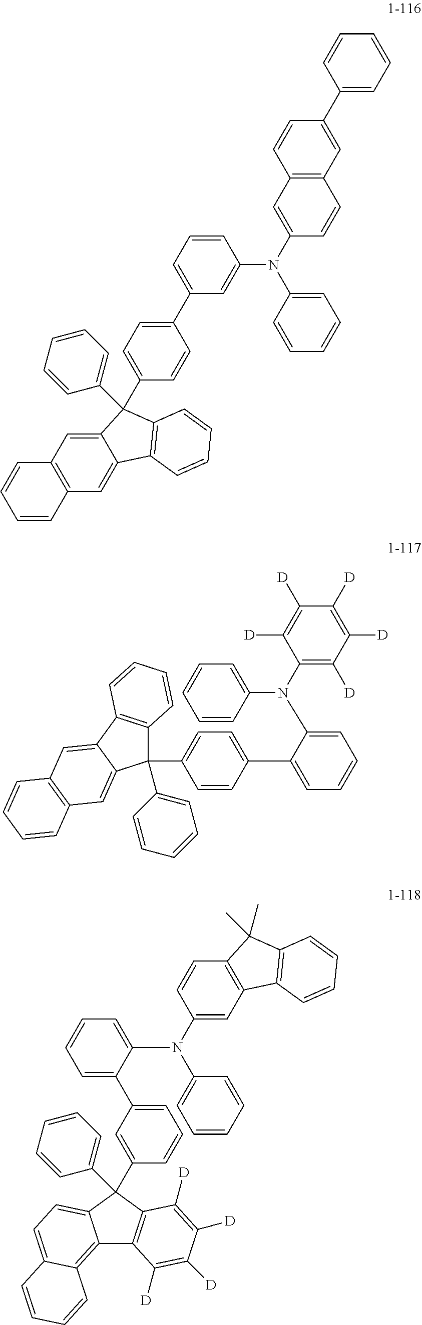 [SMILES: c1ccc(-c2ccc3cc(N(c4ccccc4)c4cccc(-c5ccc(C6(c7ccccc7)c7ccccc7-c7cc8ccccc8cc76)cc5)c4)ccc3c2)cc1 ; [2H]c1c([2H])c([2H])c(N(c2ccccc2)c2ccccc2-c2ccc(C3(c4ccccc4)c4ccccc4-c4cc5ccccc5cc43)cc2)c([2H])c1[2H] ; [2H]c1c([2H])c([2H])c2c(c1[2H])-c1c(ccc3ccccc13)C2(c1ccccc1)c1cccc(-c2ccccc2N(c2ccccc2)c2ccc3c(c2)-c2ccccc2C3(C)C)c1]

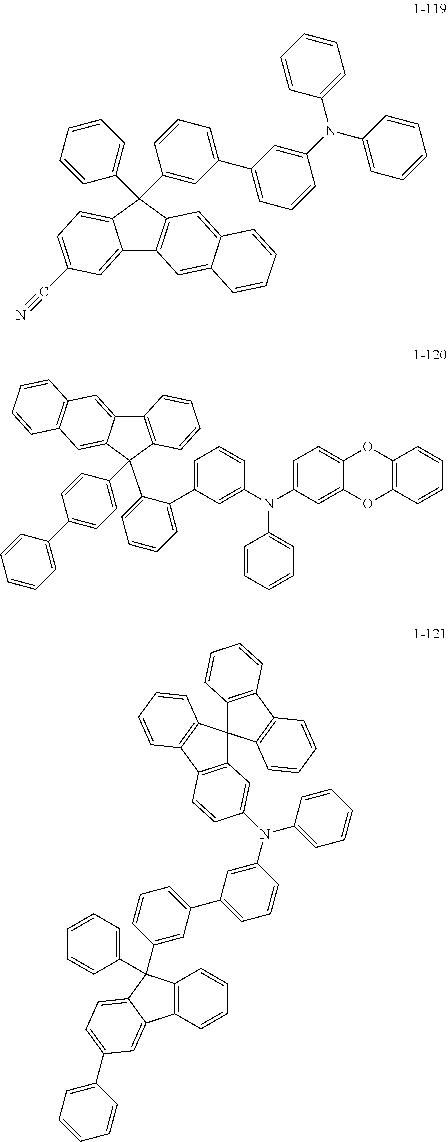 [SMILES: N#Cc1ccc2c(c1)-c1cc3ccccc3cc1C2(c1ccccc1)c1cccc(-c2cccc(N(c3ccccc3)c3ccccc3)c2)c1 ; c1ccc(-c2ccc(C3(c4ccccc4-c4cccc(N(c5ccccc5)c5ccc6c(c5)Oc5ccccc5O6)c4)c4ccccc4-c4cc5ccccc5cc43)cc2)cc1 ; c1ccc(-c2ccc3c(c2)-c2ccccc2C3(c2ccccc2)c2cccc(-c3cccc(N(c4ccccc4)c4ccc5c(c4)C4(c6ccccc6-c6ccccc64)c4ccccc4-5)c3)c2)cc1]

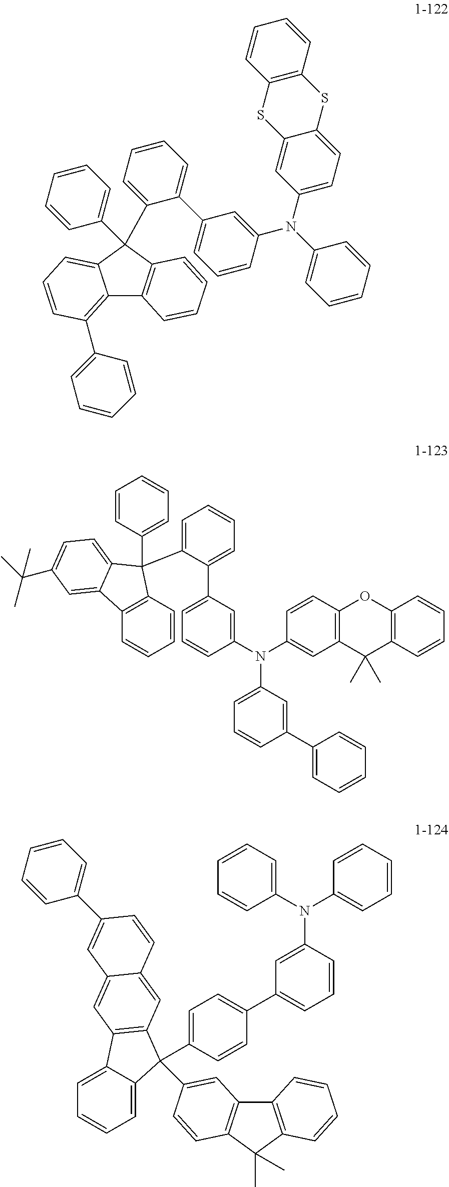 [SMILES: c1ccc(-c2cccc3c2-c2ccccc2C3(c2ccccc2)c2ccccc2-c2cccc(N(c3ccccc3)c3ccc4c(c3)Sc3ccccc3S4)c2)cc1 ; CC(C)(C)c1ccc2c(c1)-c1ccccc1C2(c1ccccc1)c1ccccc1-c1cccc(N(c2cccc(-c3ccccc3)c2)c2ccc3c(c2)C(C)(C)c2ccccc2O3)c1 ; CC1(C)c2ccccc2-c2cc(C3(c4ccc(-c5cccc(N(c6ccccc6)c6ccccc6)c5)cc4)c4ccccc4-c4cc5cc(-c6ccccc6)ccc5cc43)ccc21]

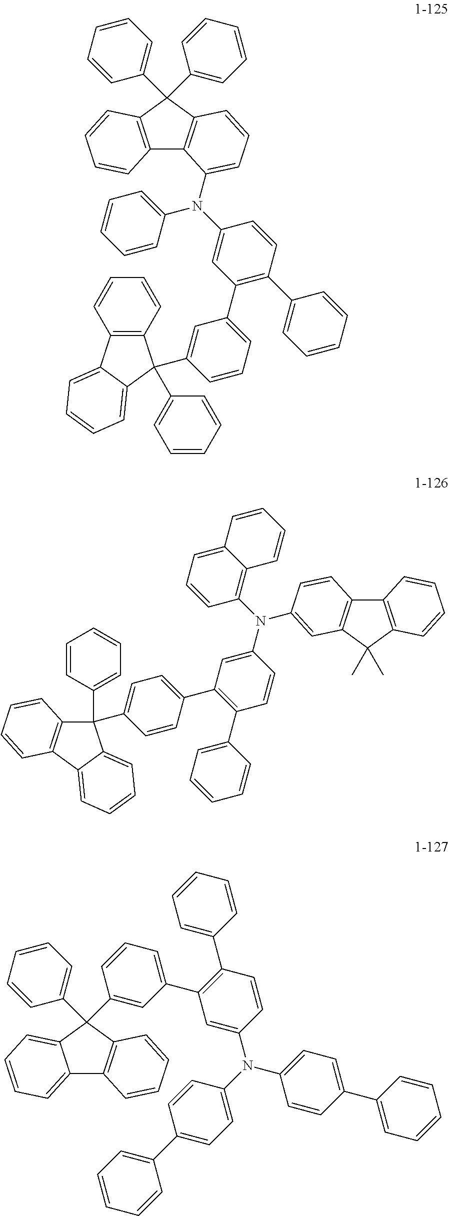 [SMILES: c1ccc(-c2ccc(N(c3ccccc3)c3cccc4c3-c3ccccc3C4(c3ccccc3)c3ccccc3)cc2-c2cccc(C3(c4ccccc4)c4ccccc4-c4ccccc43)c2)cc1 ; CC1(C)c2ccccc2-c2ccc(N(c3ccc(-c4ccccc4)c(-c4ccc(C5(c6ccccc6)c6ccccc6-c6ccccc65)cc4)c3)c3cccc4ccccc34)cc21 ; c1ccc(-c2ccc(N(c3ccc(-c4ccccc4)cc3)c3ccc(-c4ccccc4)c(-c4cccc(C5(c6ccccc6)c6ccccc6-c6ccccc65)c4)c3)cc2)cc1]

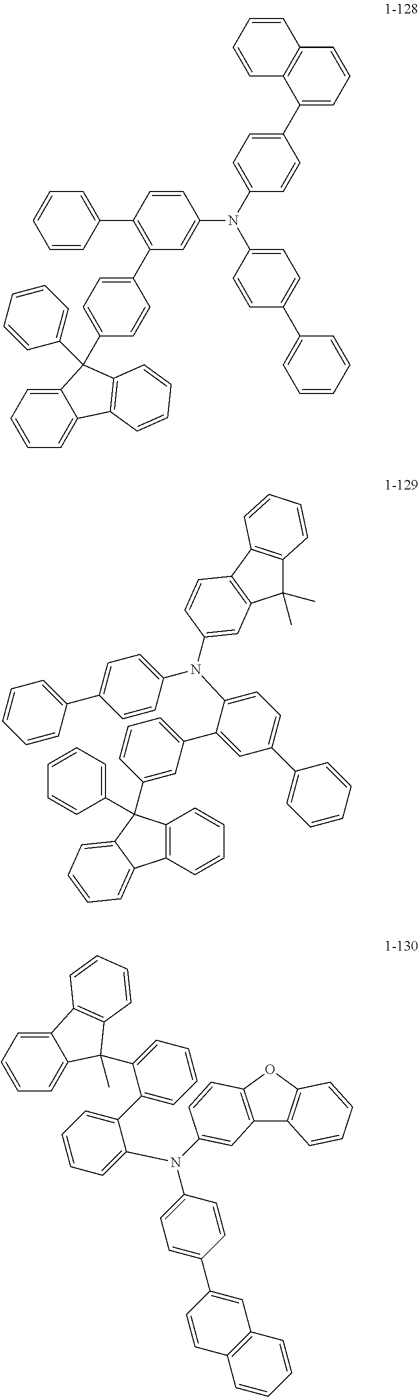 [SMILES: c1ccc(-c2ccc(N(c3ccc(-c4cccc5ccccc45)cc3)c3ccc(-c4ccccc4)c(-c4ccc(C5(c6ccccc6)c6ccccc6-c6ccccc65)cc4)c3)cc2)cc1 ; CC1(C)c2ccccc2-c2ccc(N(c3ccc(-c4ccccc4)cc3)c3ccc(-c4ccccc4)cc3-c3cccc(C4(c5ccccc5)c5ccccc5-c5ccccc54)c3)cc21 ; CC1(c2ccccc2-c2ccccc2N(c2ccc(-c3ccc4ccccc4c3)cc2)c2ccc3oc4ccccc4c3c2)c2ccccc2-c2ccccc21]

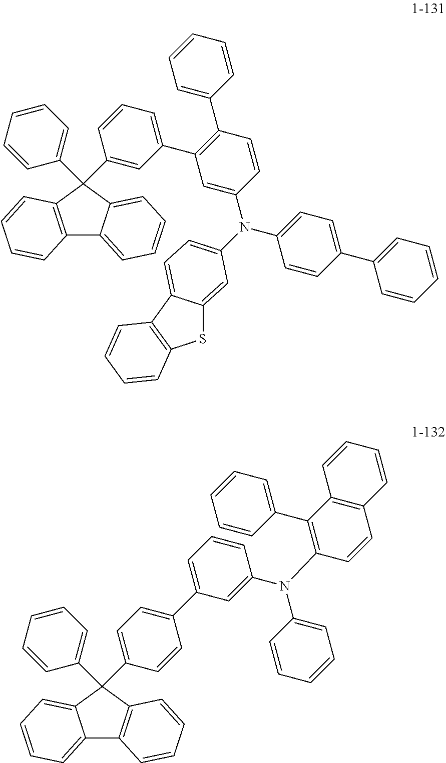 [SMILES: c1ccc(-c2ccc(N(c3ccc(-c4ccccc4)c(-c4cccc(C5(c6ccccc6)c6ccccc6-c6ccccc65)c4)c3)c3ccc4c(c3)sc3ccccc34)cc2)cc1 ; c1ccc(-c2c(N(c3ccccc3)c3cccc(-c4ccc(C5(c6ccccc6)c6ccccc6-c6ccccc65)cc4)c3)ccc3ccccc23)cc1]

7. An organic electronic element comprising an anode, a cathode, and an organic material layer formed between the anode and the cathode, wherein the organic material layer comprises a single compound or 2 or more compounds represented by Formula (1) of claim 1.

8. The organic electronic element of claim 7, wherein the organic material layer comprises at least one of a hole injection layer, a hole transport layer, an emitting auxiliary layer, an emitting layer, an electron transport auxiliary layer, an electron transport layer, and an electron injection layer.

9. The organic electronic element of claim 7, wherein the compound is used as a material of the hole transport layer.

10. The organic electronic element of claim 7, wherein the compound is used as a material of the emitting auxiliary layer.

11. The organic electronic element of claim 7, wherein the compound is used as a phosphorescent host material of the emitting layer.

12. The organic electronic element of claim 7, wherein the organic electronic element further comprises a light efficiency enhancing layer formed on at least one of the surfaces of the anode and the cathode, the surface being opposite to the organic material layer.

13. The organic electronic element of claim 7, wherein the organic material layer comprises 2 or more stacks including a hole transport layer, an emitting layer, and an electron transport layer sequentially formed on the anode.

14. The organic electronic element of claim 7, wherein the organic material layer further comprises a charge generation layer formed between the 2 or more stacks.

15. An electronic device comprising: a display device comprising the organic electronic element of claim 7; and a control unit for driving the display device.

16. An electronic device according to claim 15, wherein the organic electronic element is at least one of an OLED, an organic solar cell, an organic photo conductor (OPC), an organic transistor (organic TFT), and an element for monochromic or white illumination.
